(19) United States
(12) Patent Application Publication
(10) Pub. No.: US 2026/0176276 A1
(43) Pub. Date: Jun. 25, 2026

(54) COMPOUND FOR REGULATING AND CONTROLLING ACTIVITY OF 15-PGDH AND PREPARATION METHOD THEREFOR

(71) Applicant: SCINNOHUB PHARMACEUTICAL CO., LTD, Chengdu, Sichuan Province (CN)

(72) Inventors: Sen JI, Chengdu, Sichuan Province (CN); Hao WANG, Chengdu, Sichuan Province (CN); Wenqing ZHANG, Chengdu, Sichuan Province (CN); Ting SHEN, Chengdu, Sichuan Province (CN); Xiao WANG, Chengdu, Sichuan Province (CN); Jie LIANG, Chengdu, Sichuan Province (CN); Nan LI, Chengdu, Sichuan Province (CN); Jing WANG, Chengdu, Sichuan Province (CN); Yuanqing TANG, Chengdu, Sichuan Province (CN); Xiaodong ZHANG, Chengdu, Sichuan Province (CN); Jun TANG, Chengdu, Sichuan Province (CN)

(21) Appl. No.: 19/128,730

(22) PCT Filed: Nov. 10, 2023
(86) PCT No.: PCT/CN2023/130971

§ 371 Date: May 9, 2025

(30) Foreign Application Priority Data
Nov. 11, 2022 (CN) 202211412106.3
Oct. 18, 2023 (CN) 202311358448.6

(51) Int. Cl. C07D 487/04; C07D 401/14; C07D 413/14

(52) U.S. Cl. CPC C07D 487/04; C07D 401/14; C07D 413/14

(57) ABSTRACT

A compound of formula (I) for regulating and controlling the activity of 15-PGDH, or a stereoisomer, tautomer or mixture thereof, or a pharmaceutical salt, solvate or prodrug thereof, a pharmaceutical composition containing same and the use thereof in the inhibition of the activity of 15-PGDH.

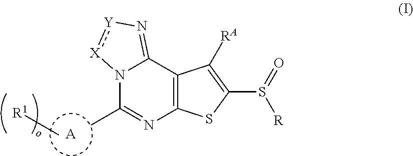

### TECHNICAL FIELD

The present application relates to a compound for regulating the activity of 15-PGDH and a preparation method thereof, specifically relates to a compound that can be used as a medicament for regulating the activity of 15-PGDH, and a pharmaceutically acceptable salt thereof, a composition comprising the compound or the salt thereof, and use thereof in the preparation of a medicament, belonging to the field of pharmaceutical chemistry.

### BACKGROUND

15-hydroxyprostaglandin dehydrogenase (15-PGDH) belongs to an evolutionarily conserved superfamily of short-chain dehydrogenases/reductases (SDRs), and has been designated as SDR36C1 according to the recently approved nomenclature for human enzymes. Based on current research results, the majority of the in vivo activity can be attributed to HPGD gene-encoded type I 15-PGDH. 15-PGDH plays an important role in the inactivation of active prostaglandins (PGD2, PGE1, PGE2, PGF2α, PGI2, etc.), hydroxyeicosatetraenoic acids (HETEs), and inflammation-resolving lipid mediators (RvD1, RvD2, RvE1, MaR1, LXA4, etc.) (hereinafter referred to generally as 15-PGDH substrates) (e.g., by catalyzing an oxidation reaction of hydroxy at position 15 of PGF2α into 15-keto-PGF2α). These 15-PGDH substrates exert their functions through specific receptors on target cells. Among others, prostaglandins, such as PGE1, PGE2, PGF2α, PGI2, etc., are often used to assess the activity of 15-PGDH. For example, the activity of PGDH is assessed by measuring ketone metabolites of hydroxy at position 15 of PGF2α (Journal of Clinical Endocrinology and Metabolism, Vol 84, No. 1, 291-299).

Receptors of 15-PGDH substrates are widely and differentially distributed in vivo, and the diversity of expression distributions, receptor types, and signaling together create a diversity of functions in vivo. For example, PGE1 acts on blood vessels and platelets to prompt an increase in blood flow by vasodilatory effects, and inhibition of platelet aggregation, and is therefore commonly used for treating diseases such as chronic arterial occlusion (thromboangiitis obliterans (TAO) or occlusive arteriosclerosis obliterans (ASO)), skin ulcers; PGF2α has uterine contraction and intraocular pressure lowering effects, and derivatives thereof have been used as therapeutic agents for glaucoma; and PGD2 inhibits inflammation by enhancing the barrier function of the pulmonary blood vessels. In addition, PGE2 has vasodilatory effects, as well as a variety of effects involving blood pressure, pain, bone formation, cell growth, stem cell differentiation, anti-fibrotic and anti-inflammatory effects, etc. PGI2 has an inhibitory effect on platelet activation and a relaxing effect on vascular smooth muscle, and its derivatives are used as therapeutic agents for chronic arterial occlusion and primary pulmonary hypertension. Inflammation-resolving lipid mediators (RvD1, RvD2, RvE1, MaR1, LXA4, etc.) inhibit migration/activation of neutrophils and accelerate apoptosis of neutrophils. In addition, it is indispensable in the process of increasing the phagocytic activity of macrophages to effectively remove apoptotic neutrophils/tissue debris remaining at the site of inflammation. These functions can promote inflammation and maintain homeostasis within the organism. These inflammation-resolving lipid mediators have been reported to show medicinal efficacy in various types of pathology models (e.g., mouse pulmonary inflammation model, colitis model, and liver injury model).

Recent studies indicate that 15-PGDH inhibitors and 15-PGDH agonists may have therapeutic values. A recent study indicates that the expression of 15-PGDH in protection against thrombin-mediated cell death is increased. It is well known that the 15-PGDH causes the inactivation of prostaglandin E2 (PGE2), and the prostaglandin E2 is a downstream product of COX-2 metabolism. Studies have shown that PGE2 is beneficial in a variety of biological processes, such as maintaining hair density and promoting skin wound healing and bone formation.

15-PGDH as an important enzyme in the inactivation of 15-PGDH substrates involves a wide range of in vivo roles, and 15-PGDH inhibitors may be used to prevent or treat diseases associated with 15-PGDH and/or 15-PGDH substrates and/or when there is a need to increase the level of 15-PGDH substrates in the body of a subject.

As mentioned above, some substrates of 15-PGDH have anti-fibrotic, anti-inflammatory, blood flow improvement, growth-promoting, stem cell increase-promoting, smooth muscle contraction/relaxation-promoting, immunosuppression and bone metabolism-affecting effects, etc. Thus, 15-PGDH inhibitors may effectively treat or prevent fibrosis (e.g., pulmonary fibrosis (idiopathic pulmonary fibrosis, etc.), hepatic fibrosis, renal fibrosis, myocardial fibrosis, scleroderma, and myelofibrosis), inflammatory diseases (e.g., chronic obstructive pulmonary disease (COPD), acute lung injuries, sepsis, lung disease and asthma, inflammatory bowel disease (e.g., ulcerative colitis and Crohn's disease), peptic ulcers (e.g., NSAID-induced ulcers), autoinflammatory diseases (e.g., Behçet's disease), vasculitis syndromes, acute liver injury, acute kidney injury, nonalcoholic steatohepatitis (NASH), atopic dermatitis, psoriasis, interstitial cystitis, prostatitis syndrome (e.g., chronic prostatitis/chronic pelvic pain syndrome)), and cardiovascular disease (e.g., pulmonary arterial hypertension, angina pectoris, myocardial infarction, heart failure, ischemic heart disease, chronic kidney disease, renal failure, cerebral apoplexy, and peripheral circulatory disorders), trauma (e.g., diabetic ulcers, burns, pressure ulcers, acute mucosal injuries (including Stevens-Johnson syndrome, mucosal injuries associated with alkylating agents, inhibitors of DNA synthesis, inhibitors of DNA gyrase, mucosal injuries associated with anticancer chemotherapeutic agents, such as antimetabolites and the like, mucosal injuries associated with cellular or humoral immunotherapy, mucosal injuries associated with graft-versus-host disease, such as mucositis or stomatitis), autoimmune diseases (e.g., multiple sclerosis or rheumatoid arthritis), graft-versus-host disease (GVHD), hair growth disorder, osteoporosis, ear diseases (e.g., hearing loss, tinnitus, vertigo, and dysequilibrium), eye diseases (e.g., glaucoma and dry eye), diabetes mellitus, underactive bladder, neutropenia, neurological diseases caused by transplantation of stem cells, bone marrows or organs (e.g., psychoneurosis, neuropathies, neurotoxic diseases, neuropathic pain, and neurodegenerative diseases), and muscle regenerative diseases (e.g., muscular dystrophy, myodystrophy, and muscle injuries); in addition, 15-PGDH inhibitors may also be used to promote cervical ripening.

Compounds and pharmaceutically acceptable salts thereof provided in the present application further meet the requirement of small molecules for inhibiting the activity of 15-PGDH.

### SUMMARY OF THE INVENTION

The present application provides a compound represented by formula (I), a stereoisomer, tautomer or mixture form thereof, or a pharmaceutically acceptable salt thereof, or a solvate (e.g., a hydrate) thereof, or a prodrug thereof.

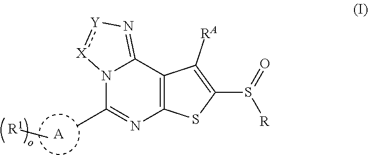

ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle;
R is selected from a C₁-C₁₀ chain hydrocarbyl, a 3-12 membered alicyclic ring, and a 3-12 membered aliphatic heterocycle, wherein R is substituted by 0-2 R², the R² is each independently selected from deuterium, tritium, nitro, hydroxy, an aldehyde group, halogen, cyano, —C(O)OR^{a}, —OC(O)R^{b}, —C(O)NHR^{X}, —NHC(O)R^{Y}, ═O, C₁-C₆ alkoxy, C₁-C₆ haloalkoxy, a 3-8 membered cycloalkyl, 3-8 membered heterocycloalkyl, a 6-10 membered aromatic ring, and a 5-10 membered aromatic heterocycle, wherein R^{a}, R^{b}, R^{X}, R^{Y} are each independently selected from C₁-C₆ alkyl, 3-8 membered cycloalkyl, and 3-8 membered heterocycloalkyl,
o is selected from 0, 1, 2, 3, and 4,
R¹ is each independently selected from deuterium, tritium, nitro, hydroxy, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl,
[symbol] is a single bond or double bond, and when [symbol] is a double bond, X and Y are each independently selected from CR^{B} or N; when [symbol] is a single bond, X and Y are each independently selected from CR^{C}R^{D}, and NR^{E},
R^{A}, R^{B}, R^{C}, R^{D}, R^{E} are each independently selected from hydrogen, hydroxy, halogen, an amine group, cyano, C₁-C₆ alkyl, C₁-C₆ alkoxy, C₁-C₆ haloalkyl, and 3-8 membered cycloalkyl,
wherein the aromatic heterocycle, the aliphatic heterocycle, the unsaturated aliphatic heterocycle, the fused ring, and the heterocycloalkyl each independently comprise 1-3 heteroatoms which are independently selected from N, O, and S,
the R¹ is optionally substituted by one or two or more independently selected from deuterium, tritium, nitro, hydroxy, an aldehyde group, an amine group, imino, halogen, cyano, an ester group, carboxyl, amido, ═O, C₁-C₆ alkyl, C₁-C₆ alkoxy, 3-8 membered cycloalkyl, a 6-10 membered aromatic ring, and a 5-10 membered aromatic heterocycle.
In certain embodiments of the present application, [symbol] is a double bond, X and Y are each independently selected from CR^{B} or N,
In certain embodiments of the present application, the [symbol] is a double bond and at least one of the X, Y is selected from CR^{B},
In certain preferred embodiments of the present application, the [symbol] is a double bond, the Y is selected from N and the X is selected from CR^{B},
In certain preferred embodiments of the present application, the is a double bond, the X is selected from N and the Y is selected from CR^{B}, or
In certain preferred embodiments of the present application, the is a double bond, both the X and the Y are selected from CR^{B},
wherein the R^{B} is each independently selected from hydrogen, hydroxy, cyano, halogen, C₃-C₈ cycloalkyl, C₁-C₆ alkyl, C₁-C₆ alkoxy, and C₁-C₆ haloalkyl; preferably, the R^{B} is selected from hydrogen, hydroxy, cyano, fluoro, chloro, bromo, methyl, ethyl, propyl, methoxy, ethoxy, trifluoromethyl, trifluoroethyl, trichloromethyl, trichloroethyl, cyclopropyl, cyclobutyl, and cyclopentyl; preferably, the R^{B} is each independently selected from hydrogen, hydroxy, cyano, fluoro, chloro, bromo, methyl, ethyl, isopropyl, methoxy, ethoxy, trifluoromethyl, trifluoroethyl, trichloromethyl, trichloroethyl, cyclopropyl, cyclobutyl, and cyclopentyl. In certain embodiments, [symbol] is a double bond, X is selected from CR^{B}, Y is selected from CR^{B} or N, wherein the R^{B} is each independently selected from hydrogen, C₁-C₆ alkyl, C₁-C₆ haloalkyl (preferably, the R^{B} is each independently selected from hydrogen, C₁-C₅ alkyl, C₁-C₅ haloalkyl; hydrogen, C₁-C₃ alkyl, C₁-C₃ haloalkyl; hydrogen, C₁-C₅ alkyl, C₁-C₅ fluoroalkyl, C₁-C₅ chloroalkyl, C₁-C₅ bromoalkyl; or hydrogen, C₁-C₃ alkyl, C₁-C₃ fluoroalkyl, C₁-C₃ chloroalkyl, C₁-C₃ bromoalkyl).
In certain embodiments of the present application, the is a single bond, and at least one of X and Y is selected from CR^{C}R^{D},
In certain preferred embodiments of the present application, the [symbol] is a single bond, and the Y is selected from NR^{E}, the X is selected from CR^{C}R^{D},
In certain preferred embodiments of the present application, the [symbol] is a single bond, and the X is selected from NR^{E}, the Y is selected from CR^{C}R^{D}, or
In certain preferred embodiments of the present application, the [symbol] is a single bond, both the X and the Y are selected from CR^{C}R^{D},
wherein the R^{C}, R^{D}, and R^{E} are each independently selected from hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl.

Further, in certain embodiments of the present application, the R^{A} is selected from hydrogen, hydroxy, cyano, fluoro, chloro, bromo, —NH₂, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl; or, R^{A} is selected from hydrogen, hydroxy, halogen (e.g., fluorine, chlorine, bromine, iodine), an amine group (e.g., —NH₂), cyano, C₁-C₆ alkyl (preferably, C₁-C₅ alkyl, C₁-C₃ alkyl), C₁-C₆ alkoxy (preferably, C₁-C₅ alkoxy, C₁-C₃ alkoxy), and C₁-C₆ haloalkyl (e.g., C₁-C₆ fluoroalkyl, C₁-C₆ chloroalkyl, C₁-C₆ bromoalkyl; preferably, C₁-C₅ haloalkyl, C₁-C₃ haloalkyl).

Further, in certain embodiments of the present application, the R is selected from C₁-C₁₀ alkyl, 3-12 membered cycloalkyl, 3-12 membered heterocycloalkyl, wherein R is substituted by 0 or 1 R².

Further, in certain embodiments of the present application, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle. Alternatively, the ring A is selected from a 6-10 membered aromatic ring, a 5-8 membered aromatic heterocycle comprising at least one heteroatom selected from N or O or S, a 5-8 membered unsaturated aliphatic heterocycle comprising at least one heteroatom selected from N or O or S, a 9-16 membered (e.g., 9-12 membered) fused ring consisting of the aromatic ring and the unsaturated aliphatic heterocycle, an 8-14 membered (e.g., 8-12 membered) fused ring consisting of the aromatic heterocycle and the unsaturated aliphatic heterocycle.

The present application further provides a compound represented by formula (II), a stereoisomer, tautomer or mixture form thereof, or a pharmaceutically acceptable salt thereof, or a solvate (e.g., a hydrate) thereof, or a prodrug thereof.

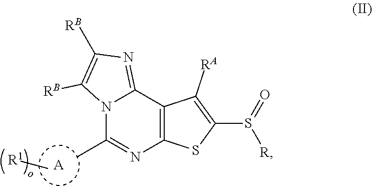

R^{B} is each independently selected from hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl,
R is selected from C₁-C₁₀ alkyl, 3-12 membered cycloalkyl, and 3-12 membered heterocycloalkyl, wherein R is substituted by 0 or 1 R², the R² is selected from deuterium, tritium, nitro, hydroxy, an aldehyde group, halogen, cyano, —C(O)OR^{a}, —OC(O)R^{b}, —C(O)NHR^{X}, —NHC(O)R^{Y}, ═O, C₁-C₆ alkoxy, C₁-C₆ haloalkoxy, 3-8 membered cycloalkyl, 3-8 membered heterocycloalkyl, a 6-10 membered aromatic ring, and a 5-10 membered aromatic heterocycle, wherein R^{a}, R^{b}, R^{X}, and R^{Y} are each independently selected from C₁-C₆ alkyl, 3-8 membered cycloalkyl, and 3-8 membered heterocycloalkyl,
R^{A} is selected from hydrogen, deuterium, tritium, hydroxy, cyano, fluorine, chlorine, bromine, —NH₂, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl,
o is selected from 0, 1, 2, 3, and 4,
R¹ is each independently selected from deuterium, tritium, nitro, hydroxy, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl,
ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle;
in certain embodiments of the present application, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle.

The present application further provides a compound represented by formula (III), a stereoisomer, tautomer or mixture form thereof, or a pharmaceutically acceptable salt thereof, or a solvate (e.g., a hydrate) thereof, or a prodrug thereof.

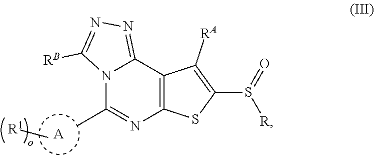

R^{B} is selected from hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl,
R is selected from C₁-C₁₀ alkyl, 3-12 membered cycloalkyl, and 3-12 membered heterocycloalkyl, wherein R is substituted by 0 or 1 R², the R² is selected from deuterium, tritium, nitro, hydroxy, an aldehyde group, halogen, cyano, —C(O)OR^{a}, —OC(O)R^{b}, —C(O)NHR^{X}, —NHC(O)R^{Y}, ═O, C₁-C₆ alkoxy, C₁-C₆ haloalkoxy, 3-8 membered cycloalkyl, 3-8 membered heterocycloalkyl, a 6-10 membered aromatic ring, and a 5-10 membered aromatic heterocycle, wherein R^{a}, R^{b}, R^{X}, and R^{Y} are each independently selected from C₁-C₆ alkyl, 3-8 membered cycloalkyl, and 3-8 membered heterocycloalkyl,
R^{A} is selected from hydrogen, deuterium, tritium, hydroxy, cyano, fluoro, chloro, bromo, —NH₂, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl,
o is selected from 0, 1, 2, 3, and 4,
R¹ is each independently selected from deuterium, tritium, nitro, hydroxy, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl,
ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle.

In certain embodiments of the present application, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle.

The present application further provides a compound represented by formula (IV), a stereoisomer, tautomer or mixture form thereof, or a pharmaceutically acceptable salt thereof, or a solvate (e.g., a hydrate) thereof, or a prodrug thereof:

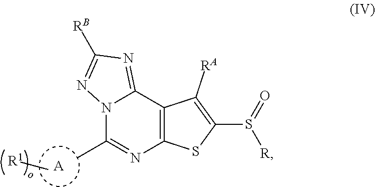

R^{B} is independently selected from hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl,
R is selected from C₁-C₁₀ alkyl, 3-12 membered cycloalkyl, and 3-12 membered heterocycloalkyl, wherein R is substituted by 0 or 1 R², the R² is selected from deuterium, tritium, nitro, hydroxy, an aldehyde group, halogen, cyano, —C(O)OR^{a}, —OC(O)R^{b}, —C(O)NHR^{X}, —NHC(O)R^{Y}, ═O, C₁-C₆ alkoxy, C₁-C₆ haloalkoxy, 3-8 membered cycloalkyl, 3-8 membered heterocycloalkyl, a 6-10 membered aromatic ring, and a 5-10 membered aromatic heterocycle, wherein R^{a}, R^{b}, R^{X}, and R^{Y} are each independently selected from C₁-C₆ alkyl, 3-8 membered cycloalkyl, and 3-8 membered heterocycloalkyl,
R^{A} is selected from hydrogen, deuterium, tritium, hydroxy, cyano, fluoro, chloro, bromo, —NH₂, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl,
o is selected from 0, 1, 2, 3, and 4,
R¹ is each independently selected from deuterium, tritium, nitro, hydroxy, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl,
ring A is selected from an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle.

In certain embodiments of the present application, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle.

The present application provides the compounds represented by formula (I), (II), (III), or (IV), stereoisomers, tautomers or mixture forms thereof, or pharmaceutically acceptable salts thereof, or solvates thereof, or prodrugs thereof,
in certain specific embodiments, the R is selected from C₁-C₁₀ alkyl, 3-12 membered cycloalkyl, and 3-12 membered heterocycloalkyl, the alkyl is a linear or branched alkyl, the cycloalkyl and the heterocycloalkyl are monocyclic or bicyclic, and the heterocycloalkyl comprises 1 heteroatom which is selected from N, O, and S, wherein R is substituted by 0 or 1 R², wherein the definition of R² is consistent with those described above;
preferably, the R is selected from C₁-C₁₀ alkyl (e.g., C₁-C₆ alkyl, C₁-C₅ alkyl), 3-8 membered cycloalkyl (e.g., 4-6 membered cycloalkyl, 5-6 membered cycloalkyl), 3-8 membered heterocycloalkyl comprising at least one heteroatom selected from N or O or S (e.g., 3-6 membered heterocycloalkyl, 4-6 membered heterocycloalkyl), wherein R is substituted by 0-2 R², the R² is each independently selected from deuterium, tritium, hydroxy, —C(O)OR^{a}, C₁-C₆ alkoxy, 3-8 membered cycloalkyl (e.g., 4-6 membered cycloalkyl, 5-6 membered cycloalkyl), 3-8 membered heterocycloalkyl comprising at least one heteroatom selected from N or O or S (e.g., 3-6 membered heterocycloalkyl, 4-6 membered heterocycloalkyl), a 6-10 membered aromatic ring (e.g., phenyl), and a 5-6 membered aromatic heterocycle comprising at least one heteroatom selected from N or O or S, wherein R^{a} is selected from C₁-C₆ alkyl (e.g., C₁-C₅ alkyl, C₁-C₃ alkyl);
preferably, the R is selected from C₁-C₁₀ alkyl (e.g., C₁-C₆ alkyl, C₁-C₅ alkyl), 4-6 membered cycloalkyl (e.g., 5-6 membered cycloalkyl), 3-6 membered heterocycloalkyl comprising at least one heteroatom selected from O or S (e.g., 4-6 membered heterocycloalkyl), wherein R is substituted by 0-1 R², the R² is each independently selected from deuterium, tritium, hydroxy, —C(O)OR^{a}, phenyl, thienyl, furanyl, pyrrolyl, thiazolyl (preferably, the R² is each independently selected from hydroxy, —C(O)OR^{a}, phenyl, thienyl), wherein R^{a} is selected from C₁-C₆ alkyl (e.g., C₁-C₅ alkyl, C₁-C₃ alkyl);
preferably, the R is selected from methyl, ethyl, n-propyl, isopropyl, n-butyl, isobutyl, tert-butyl, n-pentyl, n-hexyl, cyclopropyl, cyclobutyl, cyclopentyl, cyclohexyl, cycloheptyl, cyclooctyl,

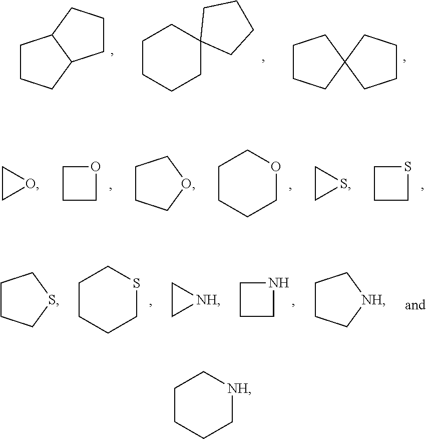

wherein R is substituted by 0 or 1 R², wherein the definition of R² is consistent with those described above. Or preferably, the R is selected from C₁-C₆ alkyl, 4-6 membered cycloalkyl (e.g., 5-6 membered cycloalkyl), and 4-6 membered heterocycloalkyl comprising 1 O atom (e.g., 4-5 membered heterocycloalkyl), wherein R is substituted by 0-1 R², the R² is each independently selected from hydroxy, and phenyl (preferably, the R² is phenyl).

Further, the R² is selected from deuterium, tritium, nitro, hydroxy, an aldehyde group, halogen, cyano, methoxy, ethoxy, n-propoxy, isopropoxy, n-butoxy, isobutoxy, tert-butoxy, trifluoromethoxy, trifluoroethoxy, trifluoropropoxy, —C(O)OCH₃, —C(O)OC₂H₅, —OC(O)CH₃, —OC(O)C₂H₅, —C(O)NHCH₃, —C(O)NHC₂H₅, —NHC(O)CH₃, —NHC(O)C₂H₅, ═O,

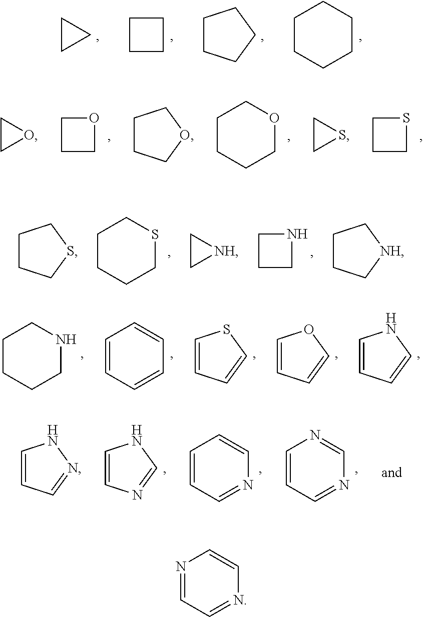

The present application further provides the compounds represented by formula (I), (II), (III), or (IV), stereoisomers, tautomers or mixture forms thereof, or pharmaceutically acceptable salts thereof, or solvates thereof, or prodrugs thereof,
in certain specific embodiments, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle, the aromatic ring and the aromatic heterocycle preferably are a monocyclic ring or a fused ring, the unsaturated aliphatic heterocycle preferably is a monocyclic ring, the fused ring preferably is a bicyclic ring, and the aromatic heterocycle, the unsaturated aliphatic heterocycle, and the fused ring each independently comprise 1 to 2 heteroatoms which are independently selected from N, O, and S;
in certain preferred embodiments of the present application, ring A is selected from phenyl, naphthyl, a 5-8 membered aromatic heterocycle comprising at least one heteroatom of N or O or S, a 5-8 membered unsaturated aliphatic heterocycle comprising at least one heteroatom of N or O or S, a 9-12 membered fused ring consisting of phenyl and the 5-8 membered unsaturated aliphatic heterocycle, and an 8-14 membered (e.g., 8-12 membered) fused ring consisting of the 5-8 membered aromatic heterocycle and the 5-8 membered unsaturated aliphatic heterocycle; preferably, ring A is selected from phenyl, naphthyl, a 5-6 membered aromatic heterocycle comprising at least one heteroatom of N or O or S, a 5-6 membered unsaturated aliphatic heterocycle comprising at least one heteroatom of N or O or S, a 9-10 membered fused ring consisting of phenyl and the 5-6 membered unsaturated aliphatic heterocycle, and an 8-10 membered fused ring consisting of the 5-6 membered aromatic heterocycle and the 5-6 membered unsaturated aliphatic heterocycle;
furthermore, the ring A is selected from

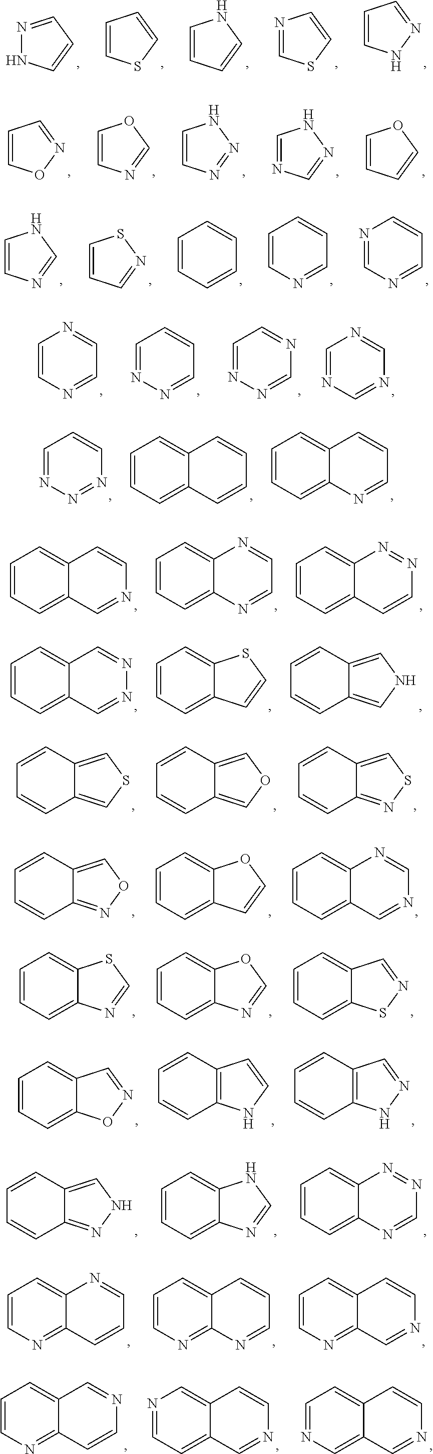

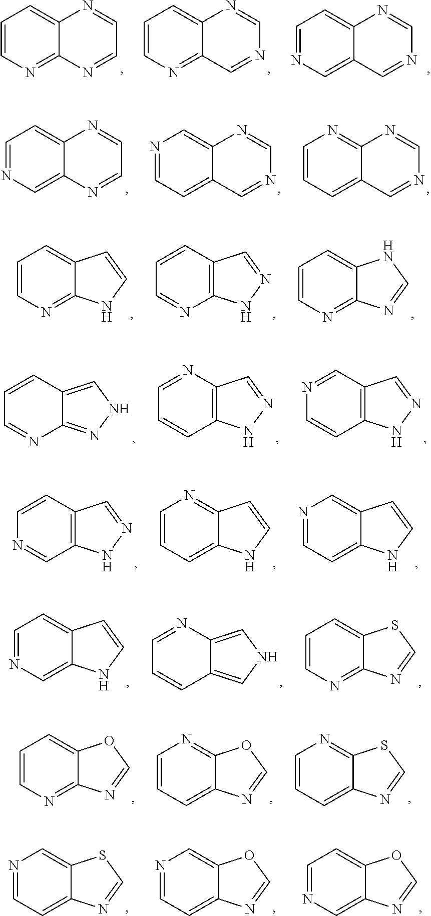

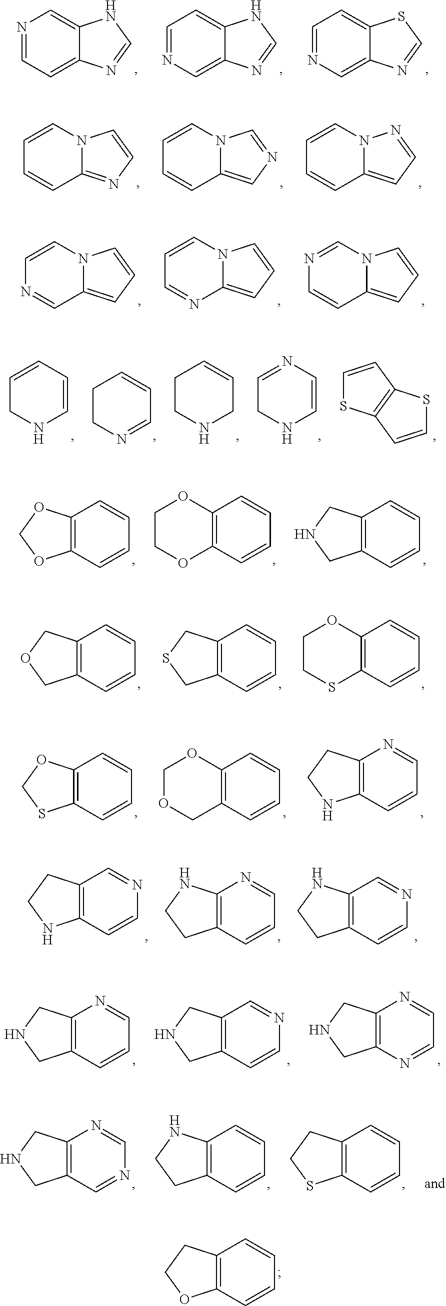

in certain specific embodiments of the present application, the ring A is preferably selected from

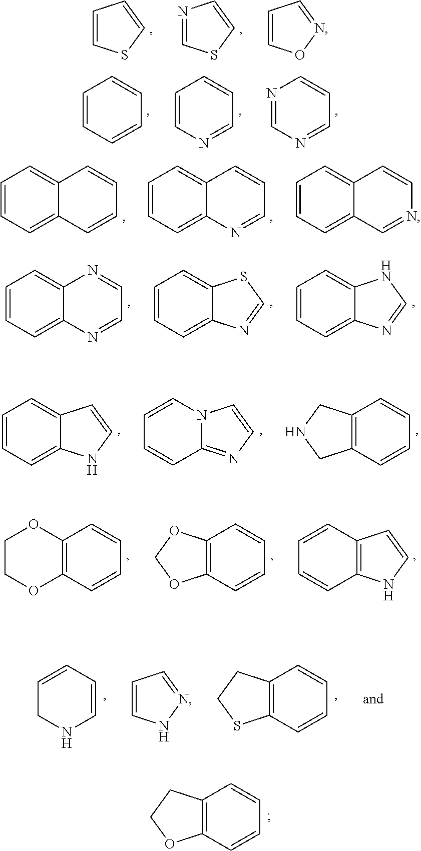

in certain specific embodiments of the present application, the ring A is selected from

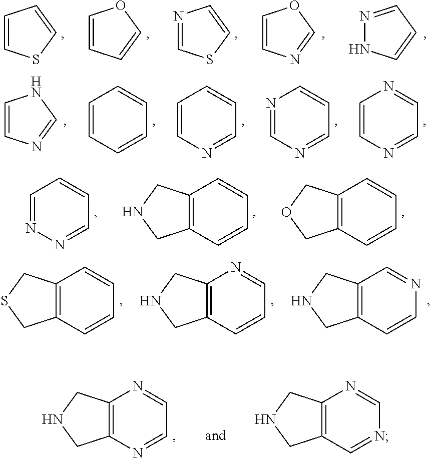

preferably, the ring A is selected from

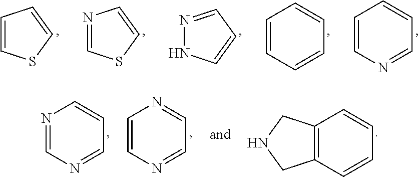

The present application provides the compounds represented by formula (I), (II), (III), or (IV), stereoisomers, tautomers or mixture forms thereof, or pharmaceutically acceptable salts thereof, or solvates thereof, or prodrugs thereof,
in some specific embodiments, the R¹ is each independently selected from deuterium, tritium, nitro, hydroxy, mercapto, halogen, cyano, ═O, imino, an amine group, an ester group, an aldehyde group, carboxyl, amido, cyclopropyl, cyclopropylmethyl, cyclobutyl, cyclopentyl, cyclohexyl, methyl, trifluoromethyl, ethyl, n-propyl, isopropyl, n-butyl, isobutyl, tert-butyl, n-pentyl, isopentyl, tert-pentyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, cyclopropoxy, cyclopropylmethoxy, n-butoxy, isobutoxy, tert-butoxy, n-pentyloxy, isopentyloxy, tert-pentyloxy, and n-hexyloxy, wherein the R¹ is optionally substituted by one or more independently selected from deuterium, tritium, nitro, hydroxy, —NH₂, mercapto, halogen, cyano, an ester group, carboxyl, amido, ═O, ═NH, C₁-C₆ alkyl, C₁-C₆ haloalkyl, C₁-C₆ alkoxy, C₁-C₆ haloalkoxy, 3-8 membered cycloalkyl, a 5-10 membered aliphatic heterocycle, a 6-10 membered aromatic ring and a 5-10 membered aromatic heterocycle.

In some specific embodiments, o is selected from 0, 1, 2, and 3 (preferably 0, 1, 2), R¹ is each independently selected from deuterium, tritium, hydroxy, ═O, imino, amine, an ester group, carboxyl, C₁-C₆ alkyl, 4-8 membered cycloalkyl (e.g., 5-6 membered cycloalkyl), and 4-8 membered heterocycloalkyl (e.g., 5-6 membered heterocycloalkyl); preferably, o is selected from 0, 1, and 2, R¹ is each independently selected from ═O, an ester group, carboxyl, and C₁-C₆ alkyl (preferably, ═O, carboxyl, C₁-C₃ alkyl).

In certain specific embodiments of the present application, the R¹ according to the present application is not substituted by other groups.

The present application provides the compounds represented by formula (I), (II), (III), or (IV), stereoisomers, tautomers or mixture forms thereof, or pharmaceutical acceptable salts thereof, or solvates thereof, or prodrugs thereof, in some specific embodiments, the o is selected from 0, 1, and 2, the R¹ is each independently selected from deuterium, tritium, ═O, —NH₂, carboxyl, C₁-C₆ alkyl, C₁-C₆ haloalkyl, C₁-C₆ alkoxy, C₁-C₆ haloalkoxy, 3-12 membered cycloalkyl, 3-12 membered heterocycloalkyl (e.g., deuterium, tritium, ═O, —NH₂, carboxyl, cyclopropyl, cyclopropylmethyl, cyclobutyl, cyclopentyl, cyclohexyl, methyl, ethyl, n-propyl, isopropyl, morpholinyl), the R^{A} is selected from —NH₂, the ring A is selected from a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle, the aromatic ring and the aromatic heterocycle are preferably a monocyclic ring or a fused ring, the unsaturated aliphatic heterocycle is preferably a monocyclic ring, the fused ring is preferably a bicyclic ring, and the aromatic heterocycle, the unsaturated aliphatic heterocycle, and the fused ring each independently comprise 1-2 heteroatoms which are independently selected from N O and S (e.g. ring A is selected from

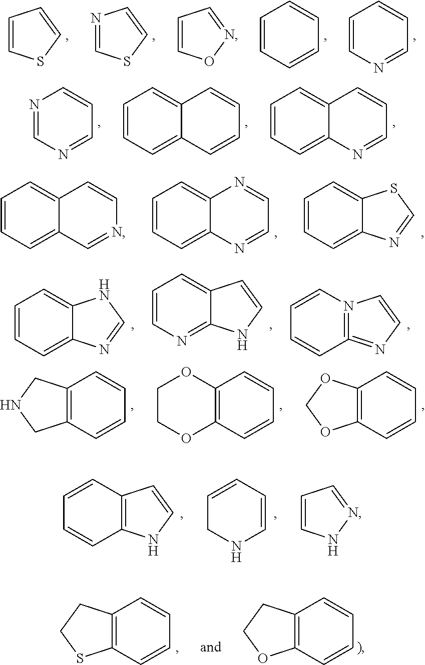

the R^{B} is each independently selected from hydrogen, deuterium, tritium, methyl, ethyl, isopropyl, and n-propyl, the R is selected from C₁-C₁₀ alkyl, 3-12 membered cycloalkyl, and 3-12 membered heterocycloalkyl, and R is optionally substituted by thienyl, phenyl, hydroxy, and —COOCH₃ (e.g., the R is selected from n-butyl, tert-butyl, isobutyl, n-pentyl, 2-methylbutyl, benzyl, hydroxyethyl, methoxycarbonylethyl, cyclopentyl, cyclohexyl, cyclobutoxy, and thenyl, preferably, n-butyl, cyclopentyl, cyclohexyl, and 2-methylbutyl).

In some embodiments, in a compound represented by formula (I), a stereoisomer, tautomer or mixture form thereof, or a pharmaceutically acceptable salt thereof, or a solvate (e.g., a hydrate) thereof, or a prodrug thereof,
the ring A is selected from a 6-10 membered aromatic ring, a 5-8 membered aromatic heterocycle comprising at least one heteroatom selected from N or O or S, a 5-8 membered unsaturated aliphatic heterocycle comprising at least one heteroatom selected from N or O or S, a 9-16 membered (e.g., 9-12 membered) fused ring consisting of the aromatic ring and the unsaturated aliphatic heterocycle, and an 8-14 membered (e.g., 8-12 membered) fused ring consisting of the aromatic heterocycle and the unsaturated aliphatic heterocycle;
the R is selected from C₁-C₁₀ alkyl (e.g., C₁-C₆ alkyl, C₁-C₅ alkyl), 3-8 membered cycloalkyl (e.g., 4-6 membered cycloalkyl, 5-6 membered cycloalkyl), and 3-8 membered heterocycloalkyl (e.g., 3-6 membered heterocycloalkyl, 4-6 membered heterocycloalkyl) comprising at least one heteroatom selected from N or O or S, wherein R is substituted by 0-2 R², the R² is each independently selected from deuterium, tritium, hydroxy, —C(O)OR^{a}, C₁-C₆ alkoxy, a 3-8 membered cycloalkyl (e.g., 4-6 membered cycloalkyl, 5-6 membered cycloalkyl), 3-8 membered heterocycloalkyl (e.g., 3-6 membered heterocycloalkyl, 4-6 membered heterocycloalkyl) comprising at least one heteroatom selected from N or O or S, a 6-10 membered aromatic ring (e.g., phenyl), and a 5-6 membered aromatic heterocycle comprising at least one heteroatom selected from N or O or S, wherein R^{a} is selected from C₁-C₆ alkyl (e.g., C₁-C₅ alkyl, C₁-C₃ alkyl);
the o is selected from 0, 1, 2, and 3 (preferably, 0, 1, 2);
the R¹ is each independently selected from deuterium, tritium, hydroxyl, ═O, imino, an amine group, an ester group, carboxyl, C₁-C₆ alkyl, 4-8 membered cycloalkyl (e.g., 5-6 membered cycloalkyl), and 4-8 membered heterocycloalkyl (e.g., 5-6 membered heterocycloalkyl);
[symbol] is a double bond, X and Y are each independently selected from CR^{B} or N, the R^{B} is each independently selected from hydrogen, halogen, C₃-C₆ cycloalkyl, C₁-C₆ alkyl, and C₁-C₆ haloalkyl (preferably, the R^{B} is each independently selected from hydrogen, C₁-C₆ alkyl, and C₁-C₆ haloalkyl);
R^{A} is selected from hydrogen, an amine group (e.g., —NH₂), hydroxy, halogen, cyano, C₁-C₆ alkyl (preferably, C₁-C₅ alkyl), C₁-C₆ alkoxy (preferably, C₁-C₅ alkoxy), and C₁-C₆ haloalkyl (e.g., C₁-C₆ fluoroalkyl, C₁-C₆ chloroalkyl, C₁-C₆ bromoalkyl).

In some preferred embodiments, in a compound represented by formula (I), a stereoisomer, tautomer or mixture form thereof, or a pharmaceutically acceptable salt thereof, or a solvate (e.g., a hydrate) thereof, or a prodrug thereof,
the ring A is selected from phenyl, naphthyl, a 5-8 membered aromatic heterocycle comprising at least one heteroatom of N or O or S, a 5-8 membered unsaturated aliphatic heterocycle comprising at least one heteroatom of N or O or S, a 9-12 membered fused ring consisting of phenyl and the 5-8 membered unsaturated aliphatic heterocycle, and an 8-14 membered (e.g., 8-12 membered) fused ring consisting of the 5-8 membered aromatic heterocycle and the 5-8 membered unsaturated aliphatic heterocycle; preferably, ring A is selected from phenyl, naphthyl, a 5-6 membered aromatic heterocycle comprising at least one heteroatom of N or O or S, a 5-6 membered unsaturated aliphatic heterocycle comprising at least one heteroatom of N or O or S, a 9-10 membered fused ring consisting of phenyl and the 5-6 membered unsaturated aliphatic heterocycle, and an 8-10 membered fused ring consisting of the 5-6 membered aromatic heterocycle and the 5-6 membered unsaturated aliphatic heterocycle;
the R is selected from C₁-C₁₀ alkyl (e.g., C₁-C₆ alkyl, C₁-C₅ alkyl), a 4-6 membered cycloalkyl (e.g., a 5-6 membered cycloalkyl), and a 3-6 membered heterocycloalkyl (e.g., a 4-6 membered heterocycloalkyl) comprising at least one heteroatom selected from 0 or S, wherein R is substituted by 0-1 R², the R² is each independently selected from deuterium, tritium, hydroxy, —C(O)OR^{a}, phenyl, thienyl, furanyl, pyrrolyl, and thiazolyl (preferably, the R² is each independently selected from hydroxy, —C(O)OR^{a}, phenyl, and thienyl), wherein R^{a} is selected from C₁-C₆ alkyl (e.g., C₁-C₅ alkyl, C₁-C₃ alkyl); for example, the R is preferably selected from butyl (e.g., n-butyl), cyclopentyl, cyclohexyl, cyclobutoxy, 1-methylbutyl, 2-methylbutyl, 3-methylbutyl, tert-butyl, 2-methylpropyl, benzyl, thenyl, methoxycarbonylethyl, hydroxyethyl, more preferably selected from n-butyl, cyclopentyl, cyclohexyl, cyclobutoxy, 2-methylbutyl, tert-butyl, and benzyl;
the o is selected from 0, 1, and 2;
the R¹ is each independently selected from ═O, an ester group, carboxyl, C₁-C₆ alkyl, and a 5-6 membered heterocycloalkyl comprising at least one heteroatom selected from N or O or S (e.g., 6 membered heterocycloalkyl comprising N and O atoms);
[symbol] is a double bond, X is selected from CR^{B}, Y is selected from CR^{B} or N, wherein the R^{B} is each independently selected from hydrogen, C₁-C₆ alkyl, and C₁-C₆ haloalkyl (the R^{B} is each independently selected from hydrogen, C₁-C₆ alkyl, C₁-C₆ fluoroalkyl, C₁-C₆ chloroalkyl, and C₁-C₆ bromoalkyl);
R^{A} is selected from hydrogen, an amine group (e.g., —NH₂), cyano, C₁-C₆ alkyl (preferably, C₁-C₅ alkyl, C₁-C₃ alkyl), C₁-C₆ alkoxy (preferably, C₁-C₅ alkoxy, C₁-C₃ alkoxy), and C₁-C₆ haloalkyl (e.g., C₁-C₆ fluoroalkyl, C₁-C₆ chloroalkyl, C₁-C₆ bromoalkyl).

In some preferred embodiments, in a compound represented by formula (I), a stereoisomer, tautomer or mixture form thereof, or a pharmaceutically acceptable salt thereof, or a solvate (e.g., a hydrate) thereof, or a prodrug thereof,
the ring A is selected from

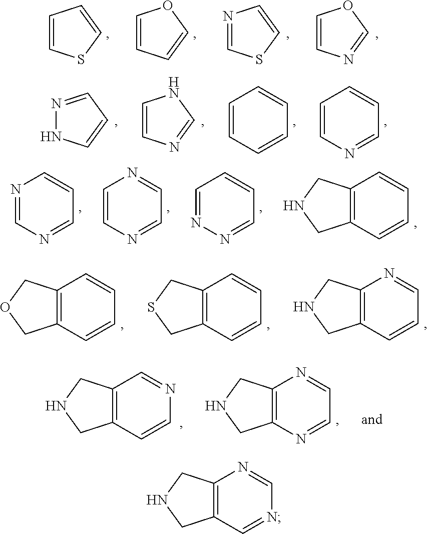

preferably, the ring A is selected from

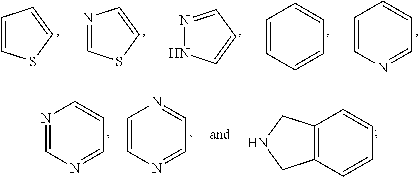

the R is selected from C₁-C₆ alkyl, a 4-6 membered cycloalkyl (e.g., a 5-6 membered cycloalkyl), and a 4-6 membered heterocycloalkyl (e.g., a 4-5 membered heterocycloalkyl) comprising 1 O atom, wherein R is substituted by 0-1 R², the R² is each independently selected from hydroxy, and phenyl (preferably, the R² is phenyl); for example, the R is preferably selected from n-butyl, cyclopentyl, cyclohexyl, cyclobutoxy, 1-methylbutyl, 2-methylbutyl, 3-methylbutyl, tert-butyl, 2-methylpropyl, benzyl, methoxycarbonylethyl, and hydroxyethyl, more preferably selected from n-butyl, cyclopentyl, cyclohexyl, cyclobutoxy, 2-methylbutyl, tert-butyl, and benzyl;
the o is selected from 0, 1, and 2;
the R¹ is each independently selected from ═O, carboxyl, C₁-C₃ alkyl, and a 5-6 membered heterocycloalkyl comprising at least one heteroatom selected from N or O or S (e.g., a 5-6 membered heterocycloalkyl comprising at least one heteroatom selected from N or O, such as a 6 membered heterocycloalkyl comprising N and O atoms);
[symbol] is a double bond, X is selected from CR^{B}, Y is selected from CR^{B} or N, wherein the R^{B} is each independently selected from hydrogen, C₁-C₆ alkyl, and C₁-C₆ haloalkyl (preferably, the R^{B} is each independently selected from hydrogen, C₁-C₅ alkyl, C₁-C₅ fluoroalkyl, C₁-C₅ chloroalkyl, and C₁-C₅ bromoalkyl; or hydrogen, C₁-C₃ alkyl, C₁-C₃ fluoroalkyl, C₁-C₃ chloroalkyl, and C₁-C₃ bromoalkyl);
R^{A} is selected from hydrogen, an amine group, cyano, C₁-C₆ alkyl (preferably, C₁-C₅ alkyl, C₁-C₃ alkyl), C₁-C₆ alkoxy (preferably, C₁-C₅ alkoxy, C₁-C₃ alkoxy), and C₁-C₆ haloalkyl (preferably, C₁-C₅ haloalkyl, C₁-C₃ haloalkyl); preferably, R^{A} is selected from hydrogen, —NH₂, C₁-C₅ alkyl (preferably, C₁-C₃ alkyl), and C₁-C₅ alkoxy (preferably, C₁-C₃ alkoxy); for example, R^{A} is selected from —NH₂, and C₁-C₅ alkyl (preferably, C₁-C₃ alkyl).

In some embodiments, each substituent in the compound of formula (II), (III), or (IV) as described above may have the same definition as described above.

In some specific embodiments of the present application, the present application provides the following compounds, stereoisomers, tautomers or mixture forms thereof, or pharmaceutically acceptable salts thereof, or solvates thereof, or prodrugs thereof:

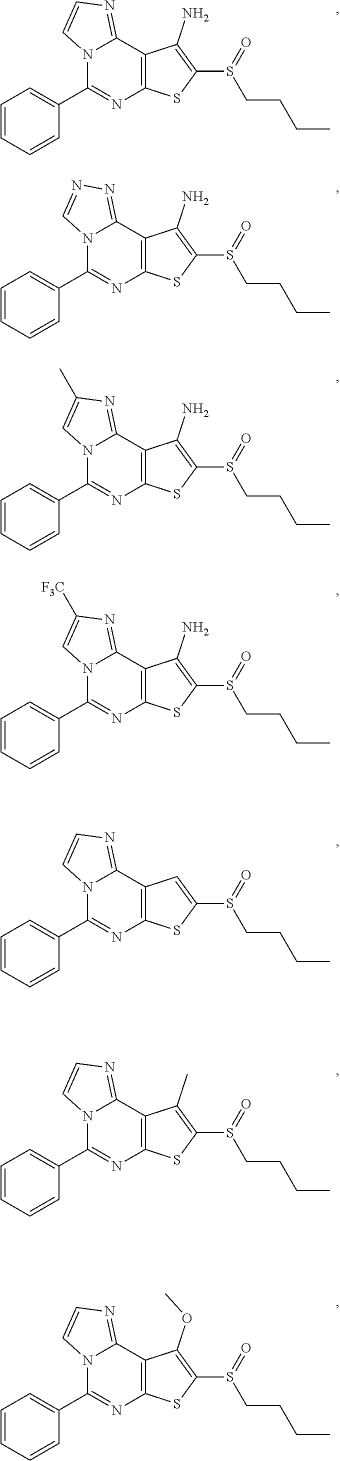

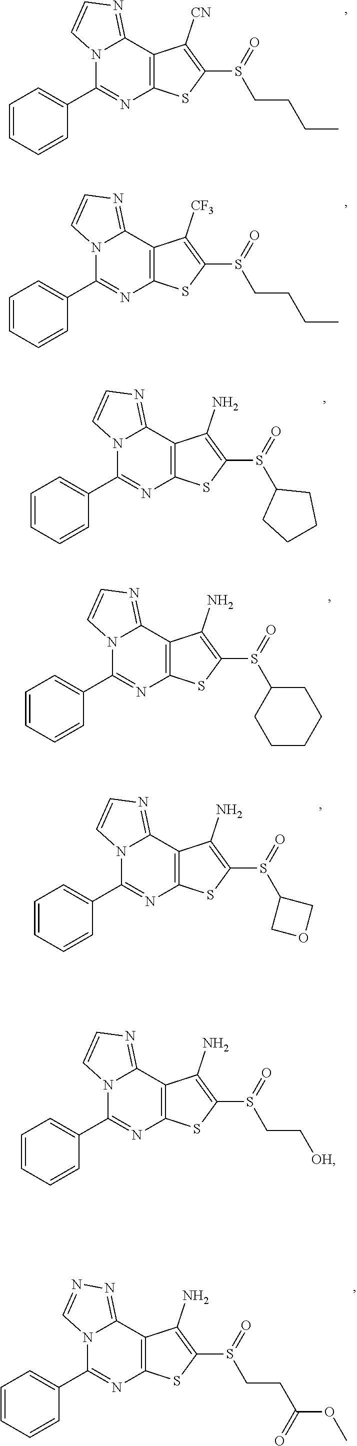

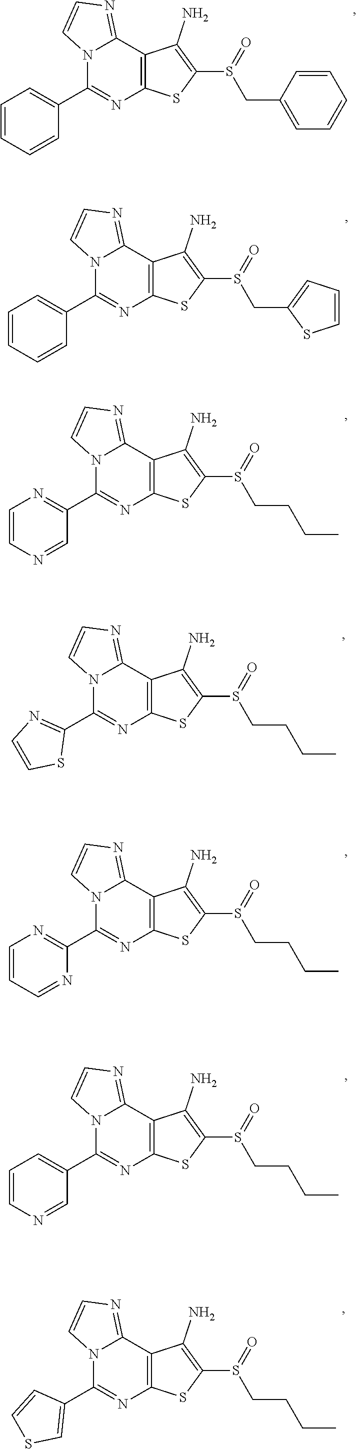

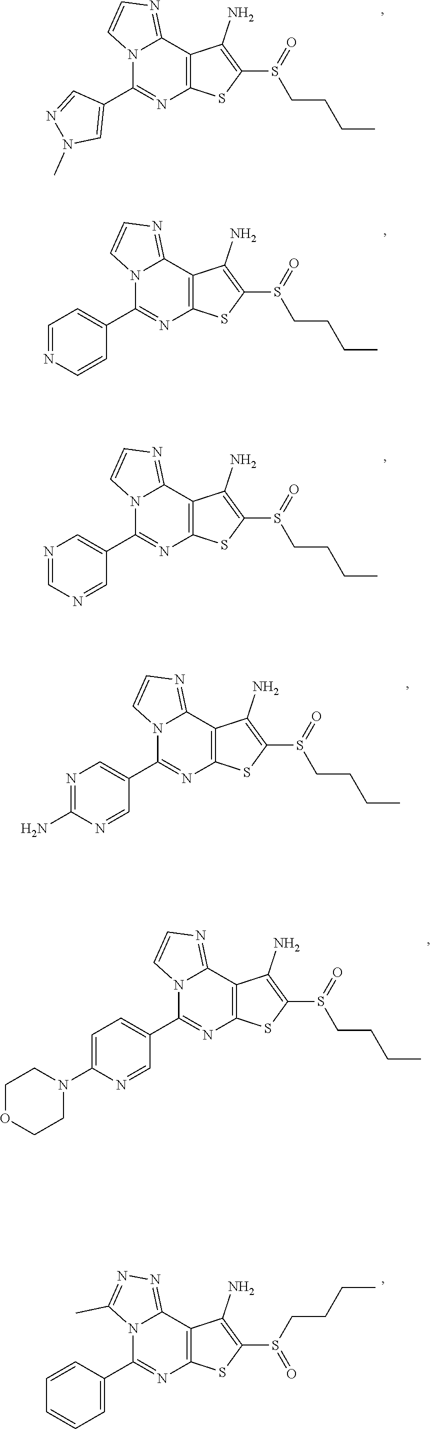 [SMILES: CCCCS(=O)c1sc2nc(-c3cnn(C)c3)n3ccnc3c2c1N ; CCCCS(=O)c1sc2nc(-c3ccncc3)n3ccnc3c2c1N ; CCCCS(=O)c1sc2nc(-c3cncnc3)n3ccnc3c2c1N ; CCCCS(=O)c1sc2nc(-c3cnc(N)nc3)n3ccnc3c2c1N ; CCCCS(=O)c1sc2nc(-c3ccc(N4CCOCC4)nc3)n3ccnc3c2c1N ; CCCCS(=O)c1sc2nc(-c3ccccc3)n3c(C)nnc3c2c1N]

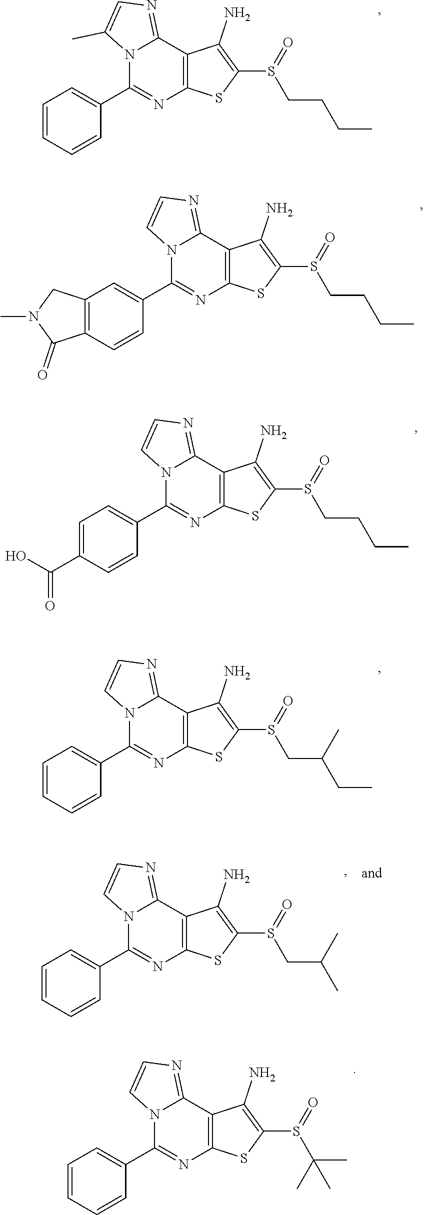

The present application further covers embodiments that are obtained by any combination, deletion or conversion of the above embodiments.

Another aspect of the present application provides a pharmaceutical composition, comprising at least one of the above-mentioned compounds, the stereoisomers, tautomers or mixture forms thereof, or the pharmaceutically acceptable salts thereof, or the solvates thereof, or the prodrugs thereof, and at least one pharmaceutically acceptable excipient.

Another aspect of the present application provides use of the above-mentioned compounds, or the stereoisomers, tautomers or mixture forms thereof, or the pharmaceutically acceptable salts thereof, or the solvates thereof, or the prodrugs thereof, or the pharmaceutical composition in the preparation of a medicament. Wherein the medicament is a 15-PGDH inhibitor which can be used for treating a disease associated with an undesirably increased activity level of 15-PGDH. Alternatively, the present application provides the above-mentioned compounds, or the stereoisomers, tautomers or mixture forms thereof, or the pharmaceutically acceptable salts thereof, or the solvates thereof, or the prodrugs thereof, or, the pharmaceutical composition for use as a medicament. Alternatively, the present application provides a method of treating or preventing a disease associated with 15-PGDH, comprising administering to a subject in need thereof the above-mentioned compounds, or the stereoisomers, tautomers or mixture forms thereof, or the pharmaceutically acceptable salts thereof, or the solvates thereof, or the prodrugs thereof, or the pharmaceutical composition. The disease associated with 15-PGDH herein refers to a disease or complication thereof for which a clinically beneficial effect, such as remission, amelioration, cessation of progression, alleviation, or no further deterioration, is achieved by inhibiting the activity of 15-PGDH.

In certain specific embodiments, the medicament or the method is used for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder; or for promoting hair growth, pigmentation, tissue repair, tissue regeneration, implant in stem cell transplantation or bone marrow transplantation or organ transplantation, neurogenesis and neuronal cell death, muscle regeneration, and cervical ripening; or for enhancing resistance to the toxicity of chemotherapy and the toxicity of immunosuppressant.

### Definition

Unless otherwise stated, the following terms used in the specification and claims have the following meanings. A particular term shall not be considered uncertain or unclear in the absence of a specific definition, but should be understood according to its ordinary meaning.

“Chain hydrocarbyl” refers to aliphatic groups consisting of only carbon and hydrogen atoms linked in a chain form. The hydrocarbyl may be a saturated hydrocarbyl or an unsaturated hydrocarbyl; the chain may be linear or branched. C₁-C₁₀ chain hydrocarbyl used in the present application refers to a linear hydrocarbyl or a branched hydrocarbyl consisting of 1-10 (e.g., 1, 2, 3, 4, 5, 6, 7, 8, 9, 10, or a range value composed of any two of the preceding numerical values) carbon atoms, including saturated hydrocarbyl and unsaturated hydrocarbyl.

“Alkyl” refers to a saturated aliphatic chain hydrocarbyl group, comprising a linear or branched alkyl. For example, C₁-C₆ alkyl used in the present application refers to linear alkyl or branched alkyl consisting of 1-6 (e.g., 1, 2, 3, 4, 5, 6, or a range value composed of any two of the preceding numerical values) carbon atoms. Typical alkyl includes, but is not limited to, methyl, ethyl, n-propyl, isopropyl, n-butyl, isobutyl, tert-butyl, n-pentyl, isopentyl, tert-pentyl, n-hexyl, etc.

“Alkoxy” refers to —O-alkyl; C₁-C₆ alkoxy used herein refers to a linear or branched alkoxy group consisting of 1 to 6 (e.g., 1, 2, 3, 4, 5, 6, or a range value composed of any two of the preceding numerical values) carbon atoms. Typical alkoxy includes, but is not limited to, methoxy, ethoxy, n-propoxy, isopropoxy, n-butoxy, isobutoxy, tert-butoxy, n-pentyloxy, isopentyloxy, tert-pentyloxy, n-hexyloxy, etc.

“Ring” refers to any cyclic covalently closed structure, including, for example, a carbocycle (e.g., an aromatic ring or an alicyclic ring) or a heterocycle (e.g., an aromatic heterocycle or an aliphatic heterocycle). The carbocycle refers to a ring only consisting of carbon atoms, and the heterocycle refers to a closed structure formed by covalently bonding carbon atoms and heteroatoms. Depending on the number of the ring, the “ring” may be monocyclic, bicyclic, tricyclic or polycyclic. When the ring is a bicyclic, tricyclic or polycyclic ring, the relationship between individual rings may include a fused ring, a spirocycle, or a bridged cycle.

“Heteroatom” refers to any atom, other than a carbon atom, that can be covalently bonded to a carbon atom. Common heteroatoms include, but are not limited to, O, S, N, P, Si, etc.

The “membered” refers to the number of skeleton atoms constituting a ring. A typical 5 membered ring may include, but is not limited to, cyclopentane, pyrrole, imidazole, thiazol, furan, thiophene, and the like; a typical 6 membered ring includes, but is not limited to, cyclohexane, pyridine, pyran, pyrazine, thiapyran, pyridazine, pyrimidines, benzene, etc.

“Alicyclic ring” or “alicyclic group” refers to a saturated or partially unsaturated aliphatic carbocyclic group. The saturated aliphatic carbocycle is referred to as, for example, a saturated alicyclic ring, and may also be referred to as a “cycloalkyl”; the partially unsaturated carbocycle may be referred to as, for example, an unsaturated alicyclic ring. The alicyclic ring may be a monocyclic ring, a spirocycle, a fused ring or a bridged cycle, for example, a 3-8 membered alicyclic ring refers to an aliphatic carbocyclic group consisting of 3-8 skeleton carbon atoms. A typical alicyclic structure includes, but is not limited to:

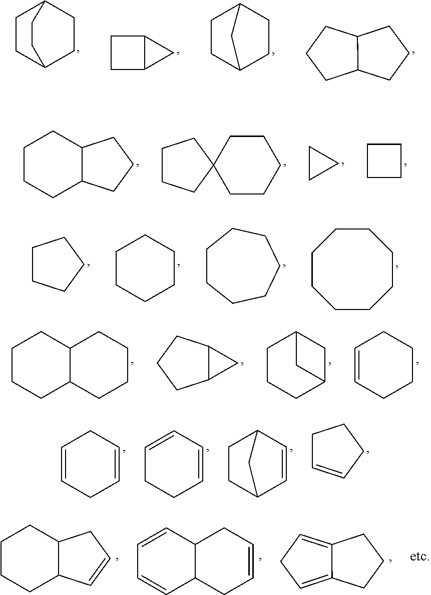

“Aliphatic heterocycle” or “aliphatic heterocyclic group” refers to a nonaromatic cyclic group formed by replacing carbon atom(s) in an alicyclic ring with one or more heteroatoms. The aliphatic heterocycle or aliphatic heterocyclic group may include a saturated aliphatic heterocycle and an unsaturated aliphatic heterocycle. For example, a 3-12 membered aliphatic heterocyclic group refers to a nonaromatic cyclic group comprising one or more heteroatoms consisting of 3-12 (e.g., 3, 4, 5, 6, 7, 8, 9, 10, 11, 12, or a range value composed of any two of the preceding numerical values) skeleton atoms, and may be a saturated aliphatic heterocyclic group and an unsaturated aliphatic heterocyclic group.

“Saturated alicyclic ring”, is also referred to as “cycloalkyl”, is an aliphatic cyclic group consisting of saturated carbon atoms as the skeleton. A 3-8 membered cycloalkyl used in the application refers to a cyclic alkyl consisting of 3-8 (e.g., 3, 4, 5, 6, 7, 8, or a range value composed of any two of the preceding numerical values) carbon atoms. A typical cycloalkyl includes, but is not limited to, cyclopropyl, cyclobutyl, cyclopentyl, cyclohexyl, bicyclo[2,1,1]hexyl, cycloheptyl, and the like.

“Saturated aliphatic heterocycle”, also known as “heterocycloalkyl”, means that the carbon atoms in the aliphatic heterocycle that form the ring skeleton are all saturated. For example, a 3-12 membered heterocycloalkyl used in the present application refers to a nonaromatic cyclic group consisting of 3-12 (e.g., 3, 4, 5, 6, 7, 8, 9, 10, 11, 12, or a range value composed of any two of the preceding numerical values) atoms constituting the ring skeleton, wherein the atoms constituting the ring skeleton consist of saturated carbon atoms and heteroatoms.

A typical saturated aliphatic heterocycle includes, but is not limited to:

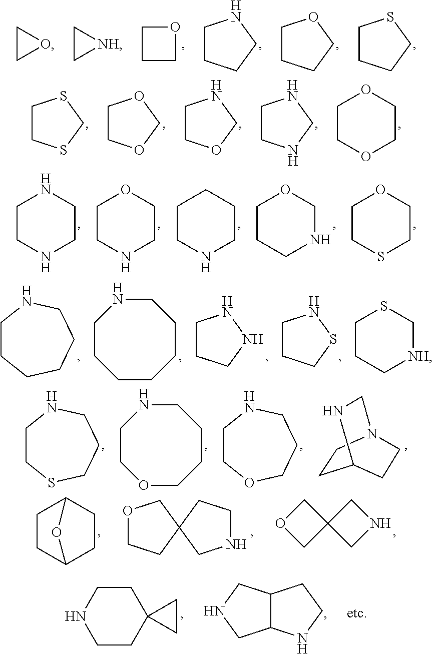

“Unsaturated aliphatic heterocycle” or “unsaturated aliphatic heterocyclyl” refers to a non-aromatic cyclic structure containing some unsaturated atoms as a ring skeleton in an aliphatic heterocycle. For example, in certain embodiments of the present application, the “unsaturated aliphatic heterocycle” means that the skeleton of the aliphatic heterocycle contains unsaturated carbon atoms. A 3-12 membered unsaturated aliphatic heterocycle used in the present application refers to a nonaromatic cyclic group consisting of 3-12 (e.g., 3, 4, 5, 6, 7, 8, 9, 10, 11, 12, or a range value composed of any two of the preceding numerical values) skeleton atoms, wherein the atoms constituting the ring skeleton include saturated carbon atoms, unsaturated carbon atoms, and heteroatoms, and a typical unsaturated aliphatic heterocycle includes, but is not limited to:

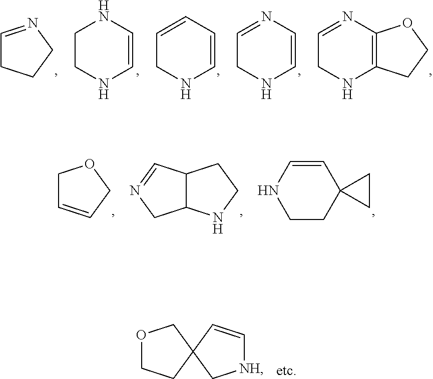

“Aromatic ring” or “aryl” refers to a completely unsaturated carbocycle with a planar ring having a delocalized 2-electron system and containing 4n+2π electrons, where n is an integer. The aromatic ring may consist of six, eight, ten, or more than ten carbon atoms, and may be a monocyclic ring or a polycyclic ring (e.g., a bicyclic ring, a tricyclic ring). Common aromatic ring includes, but is not limited to, benzene ring, naphthalene ring, phenanthrene ring, anthracene ring, tetrabenzene, pyrene ring, pentabenzene, and the like. As used in the present application, a 6-10 membered aromatic ring or a 6-10 membered aryl refers to an aromatic ring group consisting of 6-10 (e.g., 6, 7, 8, 9, 10, or a range value composed of any two of the preceding numerical values) skeleton carbon atoms.

“Aromatic heterocycle” or “heteroaryl” refers to an aromatic cyclic structure formed by replacing carbon atoms in the aromatic ring with one or more heteroatoms, and a typical aromatic heterocycle or heteroaryl includes, but is not limited to:

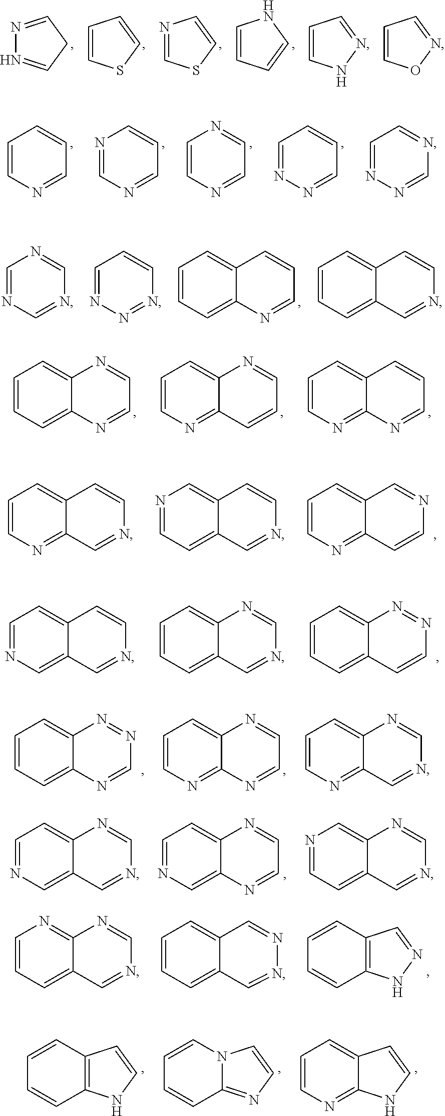

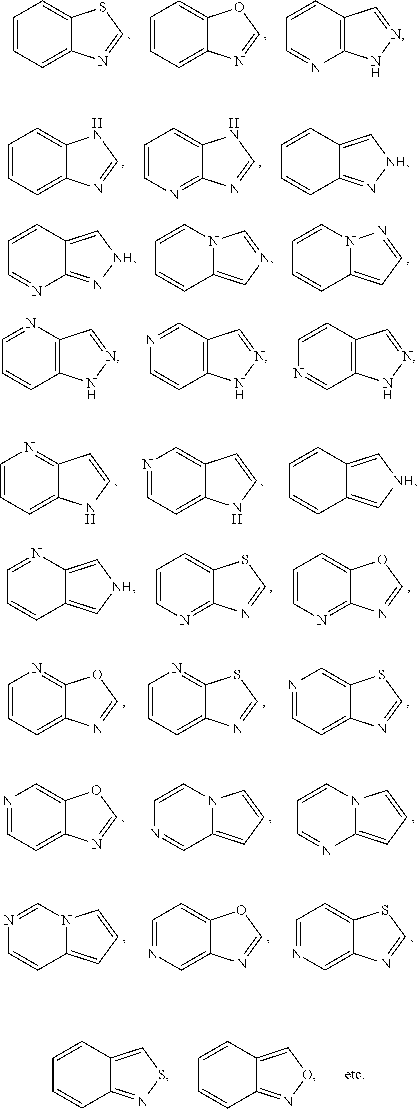

A 5-10 membered aromatic heterocycle or 5-10 membered heteroaryl used in the present application refers to an aromatic ring group comprising heteroatoms consisting of 5-10 (e.g., 5, 6, 7, 8, 9, 10, or a range value composed of any two of the preceding numerical values) skeleton atoms.

“Fused ring” refers to a cyclic structure formed by sharing two adjacent ring atoms between rings. The fused ring may be a bicyclic ring, a tricyclic ring, or a polycyclic ring.

“Fused ring consisted of an aromatic ring and an unsaturated aliphatic heterocycle” in the present application refers to a fused ring structure formed by sharing two adjacent ring atoms between the aromatic ring and the unsaturated aliphatic heterocycle; “fused ring consisted of an aromatic heterocycle and an unsaturated aliphatic heterocycle” refers to a fused ring structure formed by sharing two adjacent ring atoms between the aromatic heterocycle and the unsaturated aliphatic heterocycle. “7-12 membered fused ring consisted of an aromatic heterocycle and an unsaturated aliphatic heterocycle” in the present application refers to a fused ring structure having 7-12 (e.g., 7, 8, 9, 10, 11, 12, or a range value composed of any two of the preceding numerical values) skeleton ring atoms formed by sharing two adjacent ring atoms between the unsaturated aliphatic heterocycle and the aromatic heterocycle. “7-12 membered fused ring consisted of an aromatic ring and an unsaturated aliphatic heterocycle” in the present application refers to a fused ring structure having 7-12 (e.g., 7, 8, 9, 10, 11, 12, or a range value composed of any two of the preceding numerical values) skeleton ring atoms formed by sharing two adjacent ring atoms between the unsaturated aliphatic heterocycle and the aromatic ring.

A common fused ring consisted of an aromatic ring and an unsaturated aliphatic heterocycle includes, but is not limited to:

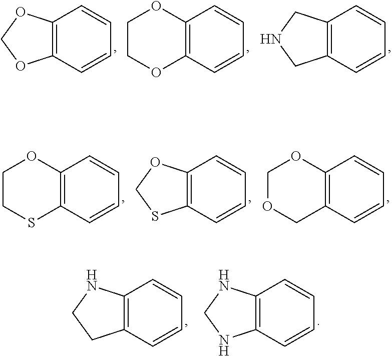

A common fused ring consisted of an aromatic heterocycle and an unsaturated aliphatic heterocycle includes, but is not limited to:

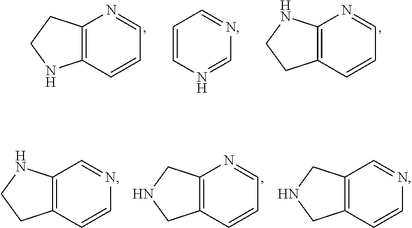

The “halogen” or “halo” refers to fluorine, chlorine, bromine or iodine.

“Haloalkyl” means that at least one hydrogen in an alkyl group is replaced by a halogen atom, and C₁-C₆ haloalkyl, as used in this application, means linear or branched alkyl consisting of 1-6 (e.g., 1, 2, 3, 4, 5, 6, or a range value composed of any two of the preceding numerical values) carbon atoms and at least one hydrogen in the alkyl is arbitrarily replaced by a halogen atom.

“Haloalkoxy” means that at least one hydrogen in an alkoxy group is replaced by a halogen atom, and a C₁-C₆ haloalkoxy, as used in this application, means a linear or branched alkoxy consisting of 1-6 (e.g., 1, 2, 3, 4, 5, 6, or a range value composed of any two of the preceding numerical values) carbon atoms and at least one hydrogen in the alkoxy is arbitrarily replaced by a halogen atom.

“Amine group” or “amine” means having a chemical structure of —NR^{S}R^{T}, wherein R^{S}, R^{T} are each independently selected from hydrogen, deuterium, tritium, alkyl, cycloalkyl.

“Imino” or “imine” means having a chemical structure of ═NR^{W}, wherein R^{W} is selected from hydrogen, deuterium, tritium, alkyl, cycloalkyl.

“Amide” or “amido” means having a chemical structure of —C(O)NR^{U}R^{V} or —NR^{U}C(O)R^{V}, wherein R^{U} and R^{V} are each independently selected from hydrogen, deuterium, tritium, alkyl, cycloalkyl, and heterocycloalkyl, and common amido includes, but is not limited to —CONH₂, —CONHCH₃, —CON(CH₃)₂, —NHCOH, —NHCOCH₃, —N(CH₃)COCH₃.

“Ester group” means having a chemical structure of a formula of —C(O)OR^{a} or —OC(O)R^{b}, wherein R^{a} and R^{b} are selected from alkyl, cycloalkyl, and heterocycloalkyl.

“Substituted” means that one or more hydrogen atoms in a group are substituted independently by a corresponding number of substituents. It goes without saying that, the substituents are only in their possible chemical positions, and those of skills in the art are able to determine (either experimentally or theoretically) possible or impossible substitutions without undue effort. For example, an amino or hydroxy with a free hydrogen may be unstable when binds to a carbon atom with an unsaturated (e.g. olefinic) bond. Each is independently selected from alkyl, cycloalkyl, aryl, heteroaryl, heterocycloalkyl, hydroxy, alkoxy, alkylthio, aryloxy, nitro, acyl, halogen, haloalkyl, amino, and the like.

The optional groups or substituents in the general formula compounds of the present application are “each independently selected from” which means that when the groups or substituents appear at multiple positions at the same time, the groups or substituents at each position may be the same or different. For example, when there are multiple RBs in the same general formula compound, each R^{B} may be the same or different and may be selected from the same or different specific groups.

“Inhibitor” refers to a substance reducing the activity of an enzyme.

“Optional” or “optionally” means that the event or circumstance subsequently described may, but not necessarily, occur, and the description includes a situation when the event or circumstance does or does not occur. For example, “optionally substituted” includes substituted or unsubstituted, e.g., “a heterocyclic group optionally substituted by an alkyl” means that the alkyl may, but not necessarily, be present, and the description includes a situation in which the heterocyclic group is substituted by the alkyl and a situation in which the heterocyclic group is not substituted by the alkyl.

“Pharmaceutical composition” indicates a mixture comprising one or more of the compounds described herein, or a physiologically/pharmaceutically acceptable salt or prodrug thereof, with other chemical components, as well as other components such as physiologically/pharmaceutically acceptable carriers and vehicles. The pharmaceutical composition is intended to facilitate administration to an organism and facilitate absorption of an active ingredient to exert the biological activity.

“Pharmaceutically acceptable” refers to those compounds, materials, compositions and/or dosage forms that are suitable for use in contact with human and animal tissues within a range of sound medical judgment, without undue toxicity, irritation, allergic response or other problems or complications, commensurate with a reasonable benefit/risk ratio.

As pharmaceutically acceptable salts, mention may be made of, e.g., metal salts, ammonium salts, salts formed with organic bases, salts formed with inorganic acids, salts formed with organic acids, salts formed with basic or acidic amino acids, and the like.

“Tautomer” or “tautomeric form” refers to structural isomers of different energies that can be interconverted through low-energy barriers. For example, proton tautomer (also known as proton transfer tautomer) includes tautomerism via proton migration, such as keto-enol and imine-enamine isomerization. A specific example of a proton tautomer is imidazole moiety, wherein the proton can migrate between the two ring nitrogens. Valence tautomers include interconversion by recombination of some bonding electrons. Non-limiting examples of tautomers include, but are not limited to,

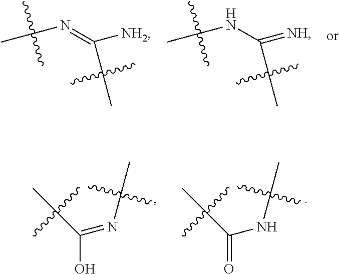 [SMILES: */N=C(\*)N ; *NC(*)=N ; */N=C(\*)O ; *NC(*)=O]

“Stereoisomer” refers to an isomer in which atoms or atomic groups within molecules are connected in the same order but have different spatial arrangements. It can be divided into several major categories: conformational isomers, cis-trans isomers, and chiral isomers. Chiral isomers can be divided into two major categories: enantiomers and diastereoisomers. The spatial arrangement of atoms in the compound structure of “stereoisomer” is usually represented by wedge-shaped covalent bonds (a solid wedge-shaped bond “ [symbol] ”, a dashed wedge-shaped bond “ [symbol] ”), wherein the solid wedge-shaped bond indicates facing out of the paper, and the dashed wedge-shaped bond indicates facing into the paper.

“Enantiomer” refers to isomerism caused by different spatial configurations of the atoms of compounds having the same molecular formula and functional groups, and the compounds form stereoisomers that are mirror images of each other and cannot overlap.

“Diastereoisomer” refers to isomerism caused by different spatial configurations of the atoms of compounds having the same molecular formula and functional groups, and the compounds are stereoisomers that do not exhibit a physical or mirror image relationship with each other. In the present application, the linear covalent bond “—” in the compound structure may indicate that it is coplanar with the paper. In the present application, when there are stereoisomers of atoms connected by a linear covalent bond, the linear covalent bond indicates that the arrangement of the connected atoms may include being coplanar with the paper, facing out of the paper, facing into the paper, or a mixture of various arrangements.

Unless otherwise indicated, the terms “comprise, comprises and comprising” or their equivalents (contain, contains, containing, include, includes, including) used herein are open-ended mode expressions, and mean that other unspecified elements, components and steps may also be covered, in addition to the elements, components and steps listed.

Unless otherwise indicated, all numbers used herein to denote amounts of ingredients, measurements, or reaction conditions should be understood to be modified in all cases by the term “about”. When associated with a percentage, the term “about” may indicate, for example, ±1%, preferably ±0.5%, more preferably ±0.1%.

Unless otherwise specified clearly in the context, singular terms herein cover plural referents, and vice versa. Similarly, unless otherwise specified clearly in the context, the word “or” herein is intended to include “and”.

Apparently, according to the above contents of the application, in accordance with the ordinary technical knowledge and means in the field, under the premise of not departing from the above basic technical concepts of the application, a variety of other forms of modifications, substitutions or changes can also be made.

The abbreviations in the application have the meanings indicated below:

| | |
|---|---|
| K₂CO₃ represents potassium carbonate; | THF represents tetrahydrofuran; |
| MeI represents iodomethane; | NaH represents sodium hydride; |
| DIPEA or DIEA represents | EtOH represents ethanol; |
| N,N-diisopropylethylamine; | |
| t-BuOK represents potassium | MeCN or ACN represents |
| tert-butoxide; | acetonitrile; |
| AcOH represents acetic acid; | CHCl₃ represents chloroform or |
| | trichloromethane; |
| TEA represents triethylamine; | TBSCI represents |
| | tert-butyldimethylchlorosilane; |
| HCl/MeOH represents methanolic | TBAF represents tetrabutylammonium |
| hydrochloric acid solution; | fluoride; |
| H₂O₂ represents hydrogen peroxide; | DMF represents N,N-dimethylformamide; |
| LiHMDS represents lithium | MeCN/H₂O represents acetonitrile/water |
| bis(trimethylsilyl)amide; | mixed solution; |
| NaSH represents sodium | m-CPBA represents m- |
| hydrosulfide; | chloroperoxybenzoic acid; |
| DCM represents dichloromethane; | bromo(chloro)methane represents |
| | bromochloromethane; |
| POCl₃ represents phosphorus | [O] represents oxidation reaction; |
| oxychloride; | |
| imidazole represents imidazole; | dioxane represents 1,4-dioxane. |

### EMBODIMENTS OF THE INVENTION

### Synthetic Methods

The present application also provides methods for synthesizing the compounds as described above. The synthesis methods of the present application are mainly based on the preparation methods reported in chemical literature or on the relevant synthesis using commercially available chemical reagents as starting materials.

### Method 1-1

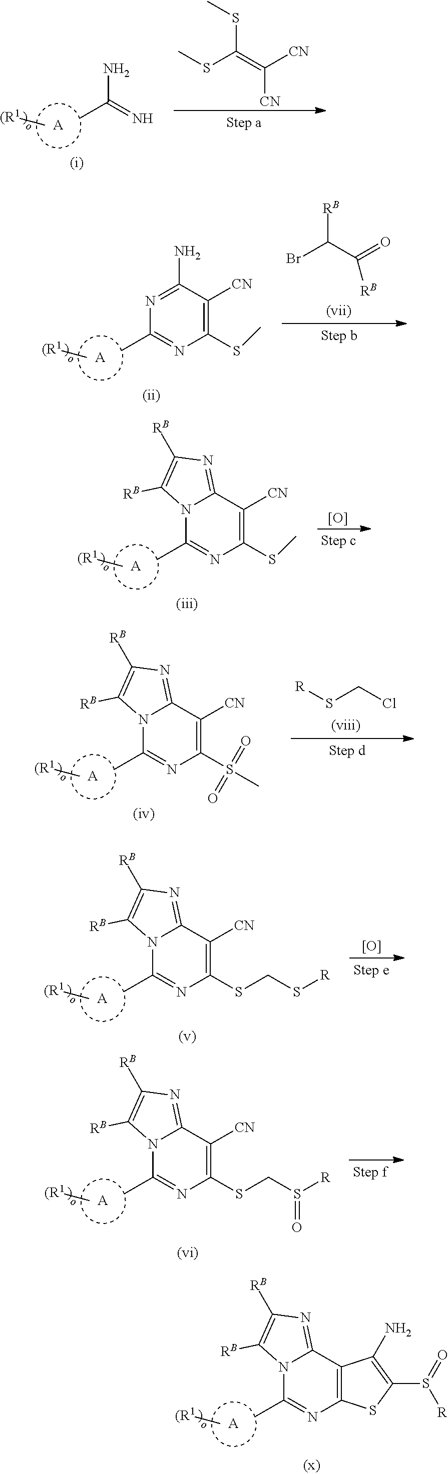

The steps are as follows:
a) a compound represented by formula (i) or a pharmaceutically acceptable salt thereof, such as the hydrochloride salt, is subjected to a ring closure reaction with bis(methylthio)methylenemalononitrile to obtain a compound represented by formula (ii);
b) a ring closure reaction is carried out with a compound represented by formula (vii) to obtain a compound represented by formula (iii);
c) the compound represented by formula (iii) is subjected to a reaction with a suitable oxidant, such as m-chloroperoxybenzoic acid, to obtain a compound represented by formula (iv) through oxidation;
d) the compound represented by formula (iv) is further subjected to a reaction with a compound represented by formula (viii) under the action of a sulfurizing agent, such as sodium hydrosulfide, to obtain a compound represented by formula (v);
e) the compound represented by formula (v) is further subjected to a reaction with a suitable oxidant, such as hydrogen peroxide, to obtain a compound represented by formula (vi) through oxidation;
f) finally, the compound represented by formula (vi) is subjected to a ring closure reaction to obtain the target compound of the present application represented by formula (x).

The compounds represented by the aforementioned formula (i), formula (vii), and formula (viii) may be commercially available products or may be prepared by the skilled in the art through the preparation methods disclosed in the prior art.

The compound represented by formula (ii) in Method 1-1 may also be prepared by a coupling reaction between 4-amino-2-chloro-6-(methylthio)pyrimidine-5-carbonitrile and

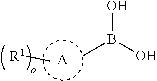

(the compound of formula (a)) under the action of a coupling reagent (e.g., 1,1-bis(diphenylphosphino)ferrocene palladium chloride). In certain embodiments, the compound of formula (a) may also be esterified with pinacol to form

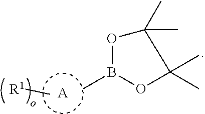

In Method 1-1, the definitions of ring A, R¹, o, R and R^{B} are consistent with those described above in the present application. In certain specific embodiments, for example, the ring A may be selected from

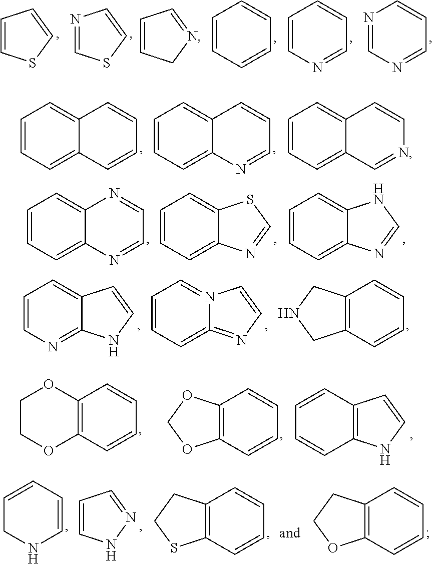

R¹ may be selected from deuterium, tritium, nitro, hydroxy, mercapto, halogen, cyano, ═O, imino, an amine group, an ester group, an aldehyde group, carboxyl, amido, cyclopropyl, cyclopropylmethyl, cyclobutyl, cyclopentyl, cyclohexyl, methyl, trifluoromethyl, ethyl, n-propyl, isopropyl, n-butyl, isobutyl, tert-butyl, n-pentyl, isopentyl, tert-pentyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, cyclopropoxy, cyclopropylmethoxy, n-butoxy, isobutoxy, tert-butoxy, n-pentyloxy, isopentyloxy, tert-pentyloxy, and n-hexyloxy; R may be selected from methyl, ethyl, n-propyl, isopropyl, n-butyl, isobutyl, tert-butyl, n-pentyl, n-hexyl, cyclopropyl, cyclobutyl, cyclopentyl, cyclohexyl, cycloheptyl, cyclooctyl,

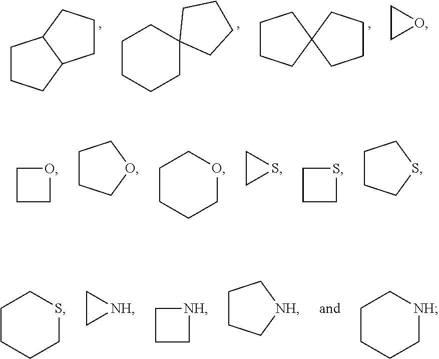

each R^{B} is independently selected from hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl; when all of R^{B} are selected from hydrogen, the compound represented by formula (vii) may also be an acetal, such as 2-bromoethyldiethylacetal.

### Method 1-2

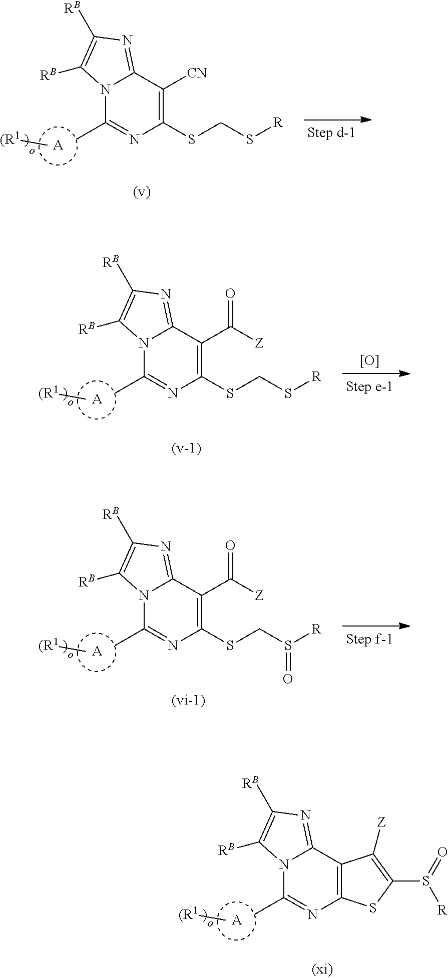

The steps are as follows:
d-1) a reaction is carried out with a compound represented by formula (v) to obtain a compound represented by formula (v-1);
e-1) the compound represented by formula (v-1) is subjected to a reaction with a suitable oxidant, such as hydrogen peroxide, to obtain a compound represented by formula (vi-1) through oxidation;
f-1) finally, the compound represented by formula (vi-1) is subjected to a ring closure reaction to obtain the target compound of the present application represented by formula (xi).

The compounds represented by the aforementioned formula (i), formula (vii), and formula (viii) may be commercially available products or may be prepared by the skilled in the art through the preparation methods disclosed in the prior art.

In Method 1-2, the definitions of ring A, R¹, o, R, and R^{B} are the same as those in Method 1-1.

The cyano of the compound represented by formula (v) in Method 1-1 is converted into group

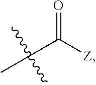

followed by oxidation in step e-1) and reduction in step f-1) to obtain the target compound represented by formula (xi) of the present application. In Method 1-2, Z is defined consistently with the definition of R^{A} described above in the present application, and Z is not —NH₂. In certain examples of the present application, for example, Z may be selected from hydrogen, cyano, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl.

### Method 2-1

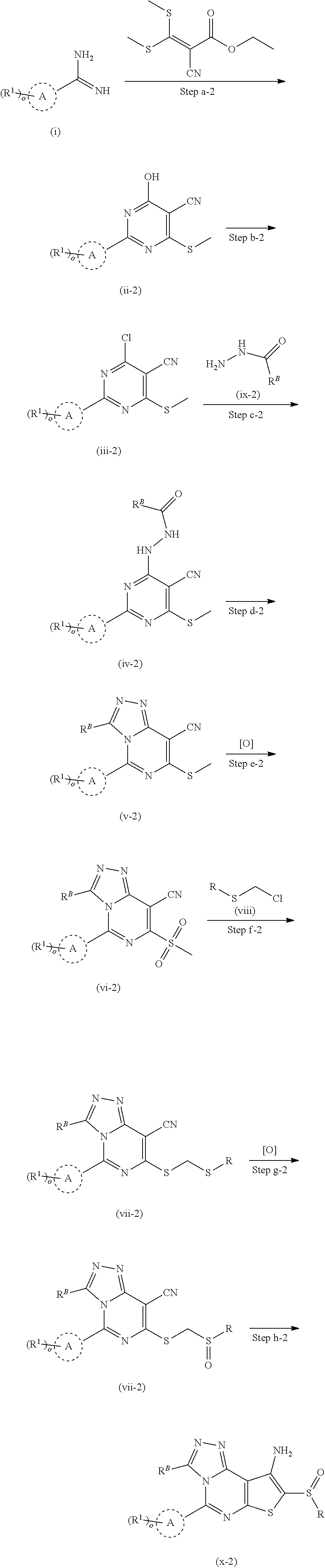

The steps are as follows:
a-2) a compound represented by formula (i) or a pharmaceutically acceptable salt thereof, such as the hydrochloride salt, is subjected to a ring closure reaction with ethyl 2-cyano-3,3-bis(methylthio)prop-2-enoate to obtain a compound represented by formula (ii-2);
b-2) a reaction is carried out under the action of a chlorinating agent such as phosphorus oxychloride, to obtain a compound represented by formula (iii-2);
c-2) a nucleophilic substitution reaction is carried out with a compound represented by formula (ix-2) to obtain a compound represented by formula (iv-2);
d-2) an intramolecular ring closure reaction is performed under the action of a dehydrating agent, such as phosphorus oxychloride, to obtain a compound represented by formula (v-2); e-2) the compound represented by formula (v-2) is subjected to a reaction with a suitable oxidant, such as m-chloroperoxybenzoic acid, to obtain a compound represented by formula (vi-2) through oxidation;
f-2) the compound represented by formula (vi-2) is further subjected to a reaction with a compound represented by formula (viii) under the action of a sulfurizing agents, such as sodium hydrosulfide, to obtain a compound represented by formula (vii-2); g-2) the compound represented by formula (vii-2) is further subjected to a reaction with a suitable oxidant, such as hydrogen peroxide, to obtain a compound represented by formula (viii-2) through oxidation;
h-2) finally, the compound represented by formula (viii-2) is subjected to a ring closure reaction to obtain the target compound of the present application represented by formula (x-2).

The compounds represented by the aforementioned formula (i), formula (ix-2), and formula (viii) may be commercially available products or may be prepared by the skilled in the art using a preparation method disclosed in the prior art.

In Method 2-1, the definitions of ring A, R¹, o, R, and R^{B} are the same as those in Method 1-1 in the present application.

The methods of synthesizing the compounds and intermediates of the present application are described below by way of example. The following examples are only intended to serve as examples of the present application, and should not be taken as a limitation to the scope of the present application. Unless otherwise indicated, the raw materials and reagents involved in the present application are all available commercially, and the specific source does not affect the implementation of the technical solution of the present application.

### Preparation Example 1: Preparation of tert-butyl-[2-(chloromethylsulfanyl)ethoxy]-dimethylsilane

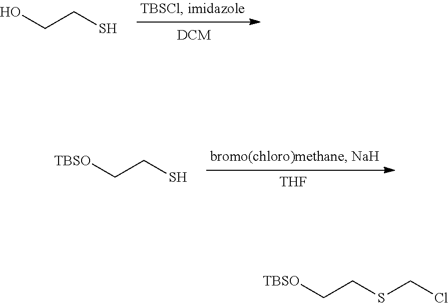

### Step 1: Preparation of 2-[tert-butyl(dimethyl)silyl]oxyethanethiol

At room temperature, 4.0 g of 2-mercaptoethanol was dissolved in 40 mL of dichloromethane, into which 8.36 g of imidazole was added, and 8.49 g of tert-butylchlorodimethylsilane was added dropwise to the reaction solution. The reaction system was stirred at room temperature for 16 hours. TLC (petroleumether:ethyl acetate=10:1) showed that the reaction was completed. The reaction solution was poured into water and extracted twice with dichloromethane. The organic phases were combined, dried over anhydrous sodium sulfate, filtered and concentrated under reduced pressure to obtain the crude product. The crude product was separated and purified by a silica gel column chromatography (petroleum ether:ethyl acetate=50:1 to 10:1) to obtain the title compound. ¹H NMR (400 MHz, CDCl₃-d) δ 3.74 (t, J=6.4 Hz, 2H), 2.64 (td, J=6.4, 8.2 Hz, 2H), 1.55 (s, 1H), 0.92-0.91 (m, 9H), 0.10-0.08 (m, 6H).

### Step 2: Preparation of tert-butyl-[2-(chloromethylsulfanyl)ethoxy]-dimethylsilane

At room temperature, 935 mg of sodium hydride was dissolved in 20 mL of tetrahydrofuran, and the reaction system was cooled to 0° C. 4.50 g of 2-[tert-butyl(dimethyl)silyl]oxyethanethiol was dissolved in 20 mL of tetrahydrofuran and added dropwise to the reaction system, which was stirred for 1 hour. At 0° C., 15.1 g of bromo(chloro)methane was added dropwise to the reaction system. The reaction system was stirred at 0° C. for 1 hour and then at 25° C. for 16 hours. TLC (petroleumether:ethyl acetate=20:1) showed that the raw materials were completely reacted. The reaction solution was filtered, the filter cake was washed with 30.0 mL of tetrahydrofuran, and the filtrate was concentrated under reduced pressure to obtain the crude product, which was obtained as the title compound. ¹H NMR (400 MHz, CDCl₃-d) δ 4.73 (s, 2H), 3.83-3.78 (m, 2H), 2.80 (t, J=6.4 Hz, 2H), 0.82 (s, 9H), 0.00 (s, 6H).

### Preparation Example 2: Preparation of Chloromethylthiocyclohexane

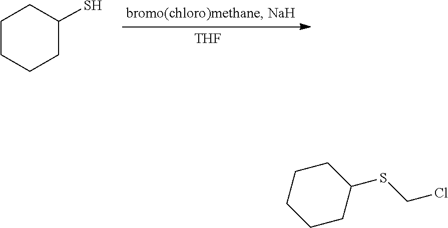 [SMILES: SC1CCCCC1 ; ClCSC1CCCCC1 ; "THF"]

At room temperature, 344 mg of sodium hydride was dissolved in 20.0 mL of tetrahydrofuran. The reaction system was cooled to 0° C., and 1.00 g of cyclohexanethiol was added dropwise to the reaction system and stirred for 1 hour. At 0° C., 5.57 g of bromo(chloro)methane was added dropwise to the reaction system. The reaction system was stirred at 0° C. for 1 hour. The reaction system was stirred at room temperature for 16 hours. GCMS showed that the raw materials were completely reacted. The reaction solution was filtered, the filter cake was washed with 10.0 mL of tetrahydrofuran, and the filtrate was concentrated under reduced pressure to obtain the crude product, which was obtained as the title compound. ¹H NMR (400 MHz, CDCl₃-d) δ 4.72 (s, 2H), 2.97-2.87 (m, 1H), 1.96 (br d, J=9.4 Hz, 2H), 1.74-1.69 (m, 2H), 1.34-1.27 (m, 4H), 1.26-1.20 (m, 2H).

### Preparation Example 3: Preparation of Chloromethylthiocyclopentane

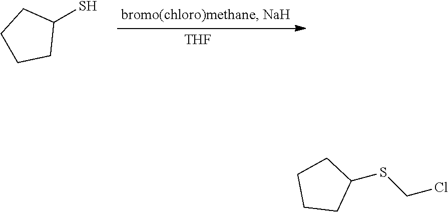 [SMILES: SC1CCCC1 ; ClCSC1CCCC1 ; "THF"]

At room temperature, 391 mg of sodium hydride was dissolved in 20.0 mL of tetrahydrofuran. The reaction system was cooled to 0° C., and 1.00 g of cyclopentanethiol was added dropwise to the reaction system and stirred for 1 hour. At 0° C., 6.33 g of bromo(chloro)methane was added dropwise to the reaction system. The reaction system was stirred at 0° C. for 2 hours. GCMS showed that the raw materials were completely reacted. The reaction solution was filtered, the filter cake was washed with 10.0 mL of tetrahydrofuran, and the filtrate was concentrated under reduced pressure to obtain the crude product, which was obtained as the title compound. GCMS m/z=150.1.

### Preparation Example 4: Preparation of 3-(chloromethylsulfanyl)oxetane

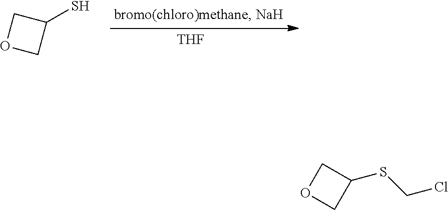 [SMILES: SC1COC1 ; ClCSC1COC1 ; "THF"]

At room temperature, 354 mg of sodium hydride was dissolved in 20.0 mL of tetrahydrofuran. The reaction system was cooled to 0° C., and 0.80 g of oxetane-3-thiol was added dropwise to the reaction system and stirred for 1 hour. At 0° C., 5.74 g of bromo(chloro)methane was added dropwise to the reaction system. The reaction system was stirred at room temperature for 16 hours. GCMS showed that the raw materials were completely reacted. The reaction solution was filtered, the filter cake was washed with 10.0 mL of tetrahydrofuran, and the filtrate was concentrated under reduced pressure to obtain the crude product, which was obtained as the title compound. ¹H NMR (400 MHz, CDCl₃-d) δ 5.03 (t, J=7.3 Hz, 2H), 4.73-4.71 (m, 2H), 4.69-4.58 (m, 2H), 4.38-4.30 (m, 1H).

### Preparation Example 5: Preparation of 2-methyl-7-methylsulfanyl-5-phenyl-imidazo [1,2-c]pyrimidine-8-carbonitrile

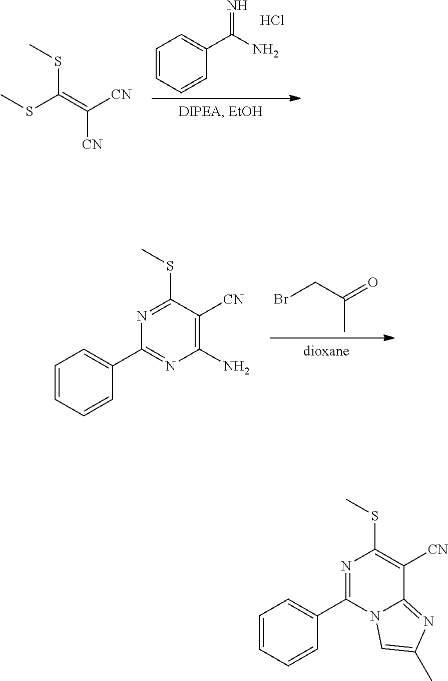

### Step 1: Preparation of 4-amino-6-(methylthio)-2-phenylpyrimidine-5-carbonitrile

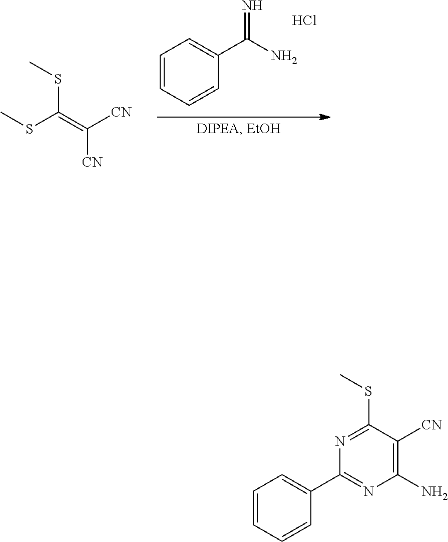

At room temperature, 9.66 g of benzamidine hydrochloride was dissolved in 100 mL of ethanol, into which 20.4 mL of N,N-diisopropylethylamine and 10.0 g of 2-[bis(methylsulfanyl)methylene]malononitrile were added. The reaction was stirred for 16 hours. LCMS showed that the raw materials were completely depleted. The reaction mixture was concentrated to remove ethanol, diluted by adding water, and extracted twice with ethyl acetate. The organic phases were combined, dried over anhydrous sodium sulfate, filtered and concentrated under reduced pressure to obtain the crude product. The obtained crude product was separated and purified by silica gel column chromatography (petroleum ether:ethyl acetate=50:1 to 10:1). The title compound was obtained. MS (ESI) m/z=243.0 (M+H)⁺, ¹H NMR (400 MHz, METHANOL-d₄) δ 8.50-8.39 (m, 2H), 7.56-7.43 (m, 3H), 2.74 (s, 3H).

### Step 2: Preparation of 7-methylsulfanyl-5-phenyl-2-(trifluoromethyl)imidazo[1,2-c]pyrimidine-8-carbonitrile

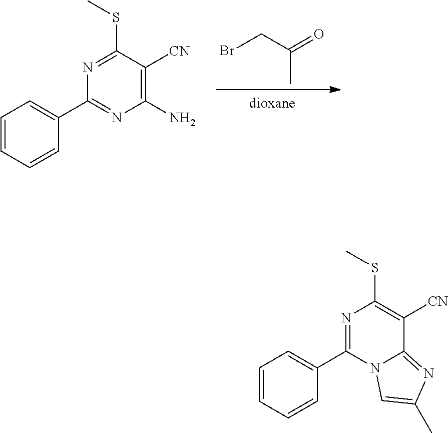

5 g of 4-amino-6-methylsulfanyl-2-phenyl-pyrimidine-5-carbonitrile was dissolved in 1,4-dioxane, into which 19.7 g of bromoacetone was added, which was then heated to 120° C., and reacted for 16 hours. TLC showed that the reaction was completed. The reaction solution was concentrated under reduced pressure to obtain the crude product. The crude product was separated using silica gel column chromatography to obtain the title compound. MS (ESI) m/z=281.4 (M+H)⁺. ¹H NMR (400 MHz, CDCl₃-d) δ 7.96 (dd, J=1.4, 8.1 Hz, 2H), 7.72-7.62 (m, 3H), 7.58 (d, J=0.8 Hz, 1H), 2.75 (s, 3H), 2.48 (d, J=0.8 Hz, 3H).

### Preparation Example 6: Preparation of 7-methylsulfanyl-5-phenyl-2-(trifluoromethyl)imidazo[1,2-c]pyrimidine-8-carbonitrile

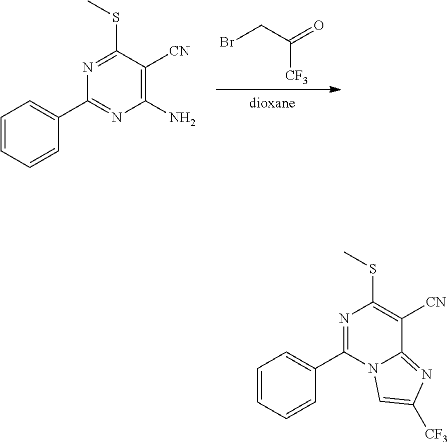 [SMILES: CSc1nc(-c2ccccc2)nc(N)c1C#N ; O=C(CBr)C(F)(F)F ; CSc1nc(-c2ccccc2)n2cc(C(F)(F)F)nc2c1C#N]

5 g of 4-amino-6-methylsulfanyl-2-phenyl-pyrimidine-5-carbonitrile was dissolved in 1,4-dioxane, into which 19.7 g of 3-bromo-1,1,1-trifluoroacetone was added, which was then heated to 120° C., and reacted for 16 hours. TLC showed that the reaction was completed. The reaction solution was concentrated under reduced pressure to obtain the crude product. The crude product was separated using silica gel column chromatography to obtain the title compound. ¹H NMR (400 MHz, DMSO-d₆) δ 8.65 (d, J=1.1 Hz, 1H), 8.08-8.02 (m, 2H), 7.73-7.66 (m, 3H), 2.81-2.74 (m, 3H).

### Preparation Example 7: Preparation of 7-methylsulfanyl-5-phenyl-imidazo[1,2-c]pyrimidine-8-carbonitrile

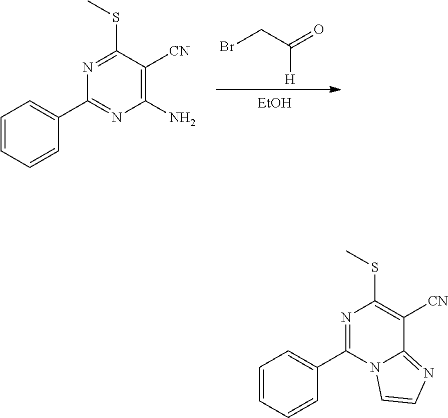

5 g of 4-amino-6-methylsulfanyl-2-phenyl-pyrimidine-5-carbonitrile was dissolved in anhydrous ethanol, into which 12.7 g of 2-bromoacetaldehyde was added, which was then heated to 120° C., and reacted for 16 hours. TLC showed that the reaction was completed. The reaction solution was concentrated under reduced pressure to obtain the crude product. The crude product was separated using silica gel column chromatography to obtain the title compound. ¹H NMR (400 MHz, CDCl₃-d) δ 7.92-7.85 (m, 2H), 7.74 (d, J=1.5 Hz, 1H), 7.64-7.52 (m, 4H), 2.68 (s, 3H).

### Preparation Example 8: Preparation of 4-amino-6-methylsulfanyl-2-(4-pyridinyl)pyrimidine-5-carbonitrile

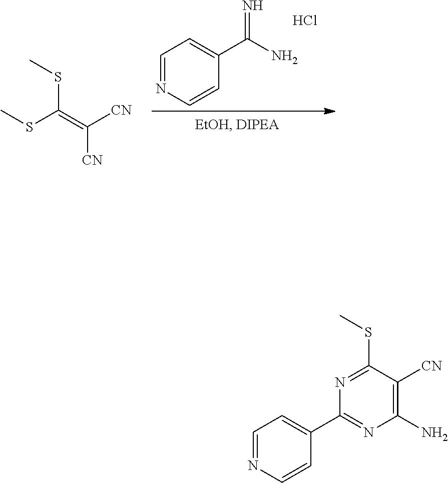

At room temperature, 20 g of 2-[bis(methylthio)methylene]malononitrile and 22.2 g of pyridine-4-carboxamidine hydrochloride were added to 200 mL of ethanol. 22.7 g of N,N-diisopropylethylamine was added to the reaction solution. After the addition was completed, the temperature was raised to 80° C., and the reaction was carried out for 12 hours. The reaction was completed as detected by LCMS. After the reaction solution was cooled to room temperature, 150 mL of water was added to the reaction solution with stirring, and the filter cake was filtered and dried to obtain the title compound. MS (ESI) m/z=244.2 (M+H)⁺, ¹H NMR (400 MHz, DMSO-d₆) δ 8.80-8.76 (m, 2H), 8.21-8.17 (m, 2H), 8.12-7.90 (m, 2H), 2.70 (s, 3H).

### Preparation Example 9: Preparation of 4-amino-6-methylsulfanyl-2-(6-morpholino-3-pyridinyl)pyrimidine-5-carbonitrile

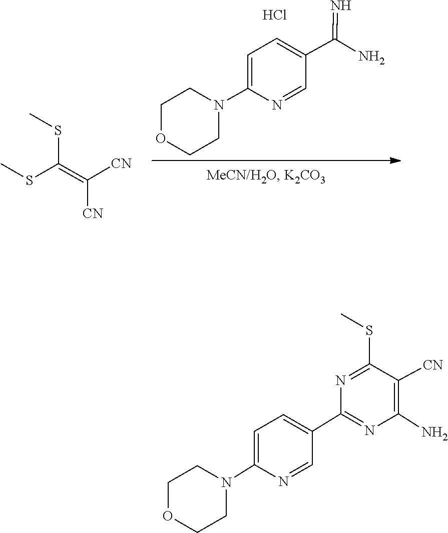

At room temperature, 12.11 g of 6-morpholinopyridine-3-carboxamidine and 8.93 g of potassium carbonate were dissolved in 120 mL of acetonitrile and 30 mL of water, into which 5 g of 2-[bis(methylsulfanyl)methylene]malononitrile was added. The mixed solution was heated to 80° C. and stirred for 16 hours. It was detected by TLC (petroleumether:ethyl acetate=3:1) that the raw materials were completely reacted. The reaction solution was cooled to 25° C., resulting in the precipitation of solids. The solids were filtered and initially rinsed with ethyl acetate. The product did not dissolve, and the filtrate was impurities. The filtrate was stored separately; then a large amount of dichloromethane (2.0 L) was used to rinse the filter cake, and the filtrate was concentrated to obtain the title compound. MS (ESI) m/z=329.0 (M+H)⁺, ¹H NMR (400 MHz, CDCl₃-d) δ 9.27 (d, J=2.3 Hz, 1H), 8.44 (dd, J=2.3, 9.0 Hz, 1H), 6.68 (d, J=9.0 Hz, 1H), 5.42 (br s, 2H), 3.89-3.82 (m, 4H), 3.72-3.66 (m, 4H), 2.71 (s, 3H).

### Preparation Example 10: Preparation of 4-amino-2-(1-methylpyrazol-4-yl)-6-methylsulfanyl-pyrimidine-5-carbonitrile

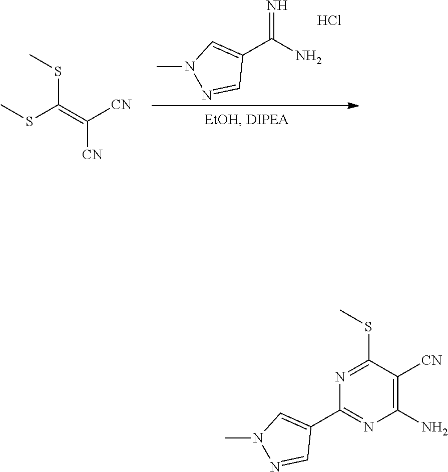

At room temperature, 11.3 g of 1-methylpyrazole-4-carboxamidine hydrochloride was dissolved in 100 mL of ethanol, into which 15.1 g of N,N-diisopropylethylamine and 10.0 g of 2-[bis(methylsulfanyl)methylene]malononitrile were added. The reaction system was heated to 80° C. and stirred for 16 hours. TLC showed that the raw materials were completely depleted. The reaction solution was cooled to room temperature (25° C.) and concentrated under reduced pressure to remove ethanol, into which 60.0 mL of water was added, and extracted with ethyl acetate three times. The organic phases were combined and washed with saturated aqueous solution of sodium chloride, and the organic phase was concentrated under reduced pressure to obtain the crude product. The obtained crude product was purified by silica gel column chromatography (petroleumether:ethyl acetate=20:1 to 0:1) to obtain the title compound. ¹H NMR (400 MHz, DMSO-d₆) δ 8.25 (s, 1H), 7.90 (s, 1H), 7.59 (br d, J=1.5 Hz, 2H), 3.87-3.81 (m, 3H), 2.44 (s, 3H).

### Preparation Example 11: Preparation of 3-methyl-7-methylsulfanyl-5-phenyl-imidazo[1,2-c]pyrimidine-8-carbonitrile

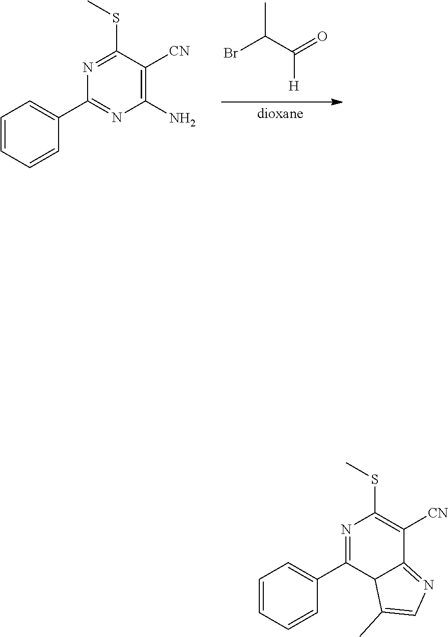

5 g of 4-amino-6-methylsulfanyl-2-phenyl-pyrimidine-5-carbonitrile was dissolved in 1,4-dioxane, into which 20.1 g of 2-bromopropionaldehyde was added, which was then heated to 120° C., and reacted for 16 hours. TLC showed that the reaction was completed. The reaction solution was concentrated under reduced pressure to obtain the crude product. The crude product was separated using silica gel column chromatography to obtain the title compound. MS (ESI) m/z=281.4 (M+H)⁺.

### Preparation Example 12: Preparation of 5-(2-methyl-1-oxoisoindolin-5-yl)-7-(methylthio)imidazo[1,2-c]pyrimidine-8-carbonitrile

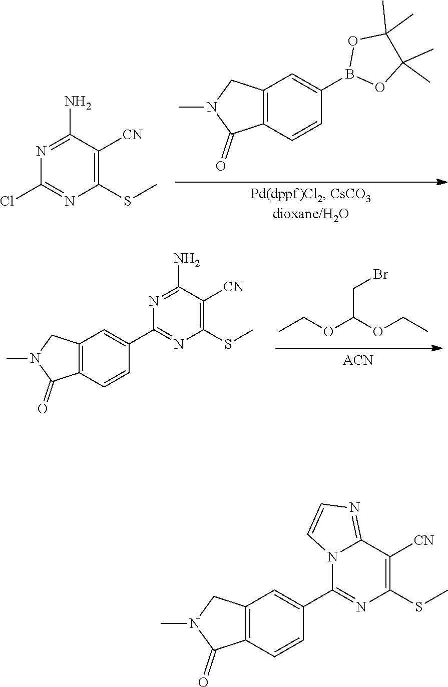

### Step 1: Preparation of 4-amino-2-(2-methyl-1-oxoisoindolin-5-yl)-6-(methylthio)pyrimidine-5-carbonitrile 4-amino-2-chloro-6-(methylthio)pyrimidine-5-carbonitrile

(1.50 g) and 2-methyl-5-(4,4,5,5-tetramethyl-1,3,2-dioxaborolan-2-yl)isoindolin-1-one (2.04 g) were added to 1,4-dioxane (20 mL) and water (2.0 mL). At 20° C., caesium carbonate (3.65 g) and 1,1-bis(diphenylphosphino)ferrocene palladium chloride (610 mg) were added under nitrogen protection. The reaction was stirred at 100° C. for 12 hours. The reaction was completed and the target product was detected. 60 mL of dimethyl sulfoxide was added to the reaction solution, and the reaction solution was stirred at 40° C. for 0.5 hours, fully dissolved, and then filtered. 200 mL of water was added to the filtrate and stirred at 15° C. for 0.5 hours. The solids precipitated out. The mixed solution was filtered, and the filter cake was rinsed twice with 50 mL water. The solids were dried to obtain the crude product. The crude product was separated and purified again using prep-HPLC to obtain the title compound. MS(ESI) m/z (M+H)⁺=312.2.

¹H NMR (400 MHz, DMSO-d₆) δ=8.70-8.23 (m, 2H), 8.13-7.65 (m, 3H), 4.59-4.54 (m, 2H), 3.11 (s, 3H), 2.72 (s, 3H).

### Step 2: Preparation of 5-(2-methyl-1-oxoisoindolin-5-yl)-7-(methylthio)imidazo[1,2-c]pyrimidine-8-carbonitrile 4-Amino-2-(2-methyl-1-oxoisoindolin-5-yl)-6-(methylthio)pyrimidine-5-carbonitrile

(0.10 g) and 2-bromo-1,1-diethoxyethane (2.62 g) were added to a microwave tube. Acetonitrile (20 mL) was added to the microwave tube. The reaction was carried out under microwave irradiation at 128° C. for 1.5 hours. TLC (petroleumether:ethyl acetate=1:1) and LCMS (EC13289-64-P1C) showed that the reaction was completed. The reaction solution was concentrated under reduced pressure to obtain the crude product, which was separated and purified by silica gel column chromatography to obtain the title compound.

MS(ESI) m/z (M+H)⁺=336.1.

### Example 1: Preparation of 8-(butylsulfinyl)-5-phenylimidazo[1,2-c]thieno[3,2-e]pyrimidin-9-amine

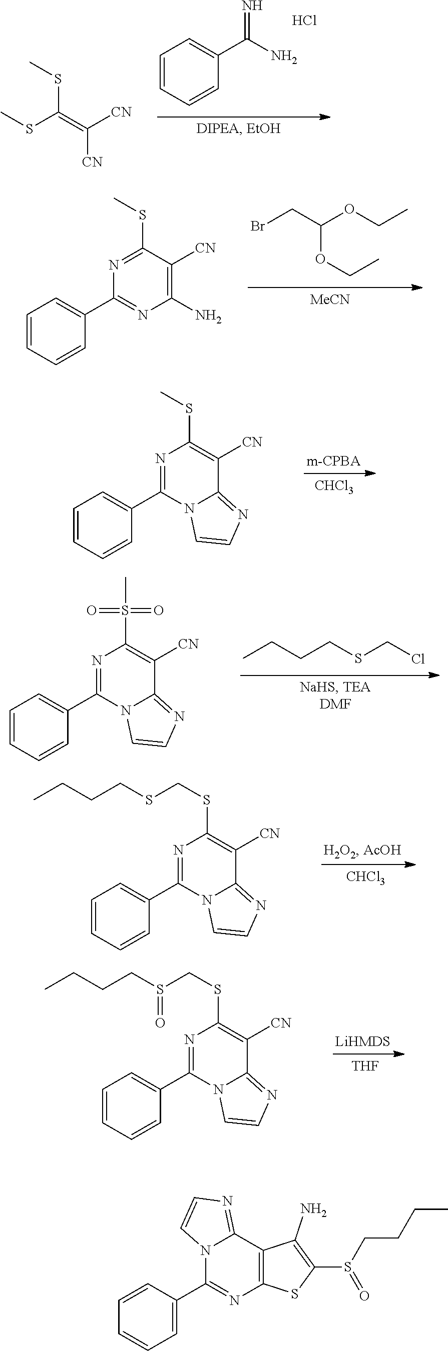

### Step 1: Preparation of 4-amino-6-(methylthio)-2-phenylpyrimidine-5-carbonitrile

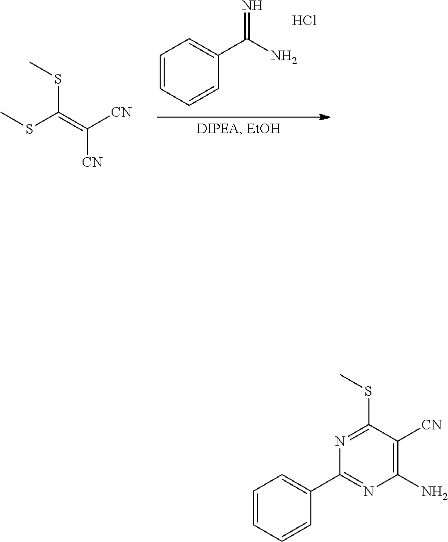

At room temperature, 9.66 g of benzamidine hydrochloride was dissolved in 100 mL of ethanol, into which 20.4 mL of N,N-diisopropylethylamine and 10.0 g of 2-[bis(methylsulfanyl)methylene]malononitrile were added. The reaction was stirred for 16 hours. LCMS showed that the raw materials were completely depleted. The reaction mixture was concentrated to remove ethanol, diluted by adding water, and extracted twice with ethyl acetate. The organic phases were combined, dried over anhydrous sodium sulfate, filtered and concentrated under reduced pressure to obtain the crude product. The obtained crude product was separated and purified by silica gel column chromatography (petroleum ether:ethyl acetate=50:1 to 10:1) to obtain the title compound.

### Step 2: Preparation of 7-methylsulfanyl-5-phenyl-imidazo[1,2-c]pyrimidine-8-carbonitrile

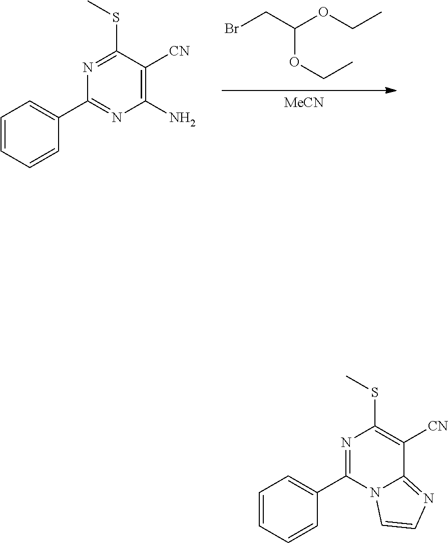

0.50 g of 4-amino-6-methylsulfanyl-2-phenyl-pyrimidine-5-carbonitrile and 1.22 g of 2-bromoethyl diethyl acetal were added to a microwave tube. 10.0 mL of ethanol was added into the microwave tube. The reaction was carried out under microwave irradiation at 120° C. for 1.5 hours. TLC showed that the reaction was completed. The reaction solution was concentrated under reduced pressure to obtain the crude product. The crude product was separated using silica gel column chromatography. The title compound was obtained. ¹H NMR (400 MHz, CDCl₃-d) δ 7.92-7.85 (m, 2H), 7.74 (d, J=1.5 Hz, 1H), 7.64-7.52 (m, 4H), 2.68 (s, 3H).

### Step 3: Preparation of 7-methylsulfonyl-5-phenyl-imidazo[1,2-c]pyrimidine-8-carbonitrile

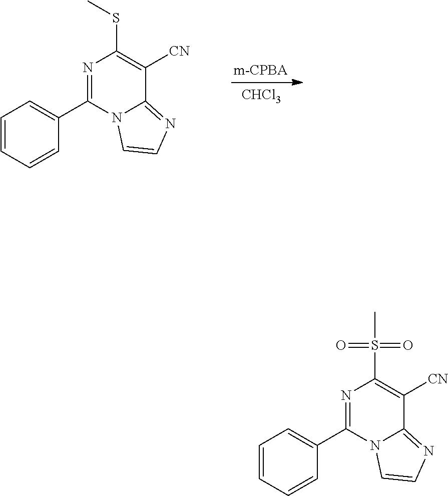 [SMILES: CSc1nc(-c2ccccc2)n2ccnc2c1C#N ; "CHCl3" ; CS(=O)(=O)c1nc(-c2ccccc2)n2ccnc2c1C#N]

At room temperature, 2.50 g of 7-methylsulfanyl-5-phenyl-imidazo[1,2-c]pyrimidine-8-carbonitrile was dissolved in 25.0 mL of chloroform, into which 4.76 g (with a content of 85.0%) of m-chloroperoxybenzoic acid was added in portions at room temperature. The reaction solution was stirred at room temperature for 16 hours. LCMS showed that the raw materials were completely depleted. The reaction solution was added to a saturated aqueous solution of sodium bicarbonate, and the aqueous phase was extracted 3 times with dichloromethane. The organic phases were combined, dried over anhydrous sodium sulfate, filtered, and concentrated under reduced pressure to obtain the crude product. The obtained crude product was separated and purified by silica gel column chromatography (petroleumether:ethyl acetate=50:1 to 10:1) to obtain the title compound. MS (ESI) m/z=298.9 (M+H)⁺, ¹H NMR (400 MHz, CDCl₃-d) δ 8.16 (d, J=1.3 Hz, 1H), 8.09-7.99 (m, 3H), 7.81-7.66 (m, 3H), 3.42 (s, 3H).

### Step 4: Preparation of 7-(butylsulfanylmethylsulfanyl)-5-phenyl-imidazo[1,2-c]pyrimidine-8-carbonitrile

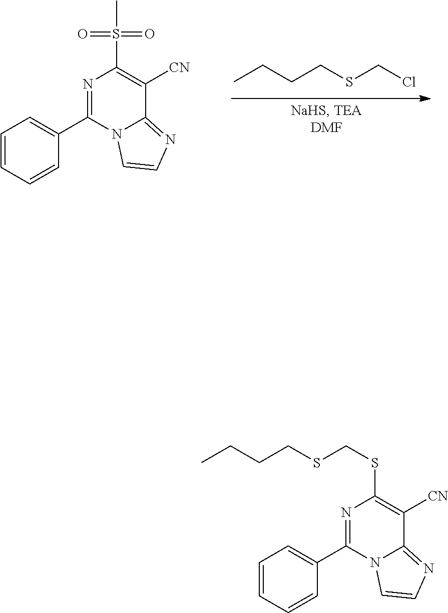

At room temperature, 0.50 g of 7-methylsulfonyl-5-phenyl-imidazole [1,2-c]pyrimidine-8-carbonitrile was added to 5.00 mL of N,N-dimethylformamide. Subsequently, 131 mg of sodium hydrosulfide was added to the reaction solution, which was then heated to 80° C. and reacted for 20 minutes. After the reaction was completed as detected by TLC, it was cooled to room temperature. Subsequently, 508 mg of triethylamine and 464 mg of 1-(chloromethylsulfinyl)butane were added to the reaction solution, which was then heated to 80° C. and reacted for 2 hours. The raw materials were completely depleted as detected by TLC and LCMS. Water was added to the reaction solution, which was then extracted twice with ethyl acetate. The organic phase was concentrated to obtain the crude product. The crude product was purified by prep-TLC (petroleumether:ethyl acetate=1:1) to obtain the title compound. MS (ESI) m/z=355.0 (M+H)⁺, ¹H NMR (400 MHz, CDCl₃-d) δ 8.01-7.96 (m, 2H), 7.86 (d, J=1.4 Hz, 1H), 7.74-7.63 (m, 4H), 4.53 (s, 2H), 2.80-2.70 (m, 2H), 1.66 (br d, J=7.1 Hz, 2H), 1.49-1.38 (m, 2H), 0.93 (t, J=7.4 Hz, 3H).

### Step 5: Preparation of 7-(butylsulfinylmethylsulfanyl)-5-phenyl-imidazo[1,2-c]pyrimidine-8-carbonitrile

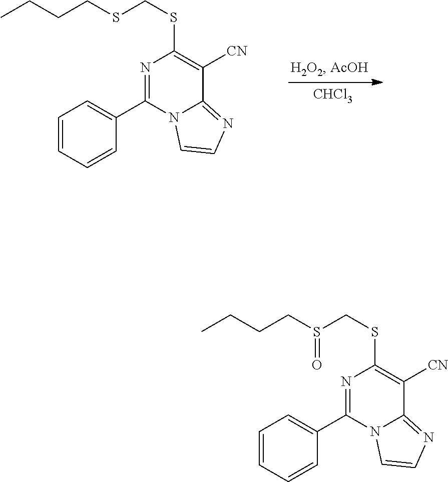 [SMILES: CCCCSCSc1nc(-c2ccccc2)n2ccnc2c1C#N ; "CHCl3" ; CCCCS(=O)CSc1nc(-c2ccccc2)n2ccnc2c1C#N]

At room temperature, 0.33 g of 7-(butylsulfanylmethylsulfanyl)-5-phenyl-imidazo[1,2-c]pyrimidine-8-carbonitrile was added to 3.30 mL of trichloromethane. Subsequently, 838 mg of acetic acid and 263 mg of hydrogen peroxide (with a content of 30%) were sequentially added to the reaction solution, which was then heated to 40° C. and reacted for 3 hours. The reaction was completed as detected by LCMS and TLC. A saturated aqueous solution of sodium bicarbonate was added to the reaction solution, which was then extracted twice with dichloromethane. The organic phase was concentrated to obtain the crude product. The crude product was purified by prep-HPLC to obtain the title compound. MS (ESI) m/z=371.3 (M+H)⁺, ¹H NMR (400 MHz, CDCl₃-d) δ 8.01-7.96 (m, 2H), 7.90 (d, J=1.5 Hz, 1H), 7.76-7.64 (m, 4H), 4.47 (d, J=13.3 Hz, 2H), 3.01-2.79 (m, 2H), 1.86-1.76 (m, 2H), 1.57-1.45 (m, 2H), 0.97 (t, J=7.4 Hz, 3H).

### Step 6: Preparation of 8-(butylsulfinyl)-5-phenylimidazo[1,2-c]thieno[3,2-e]pyrimidin-9-amine

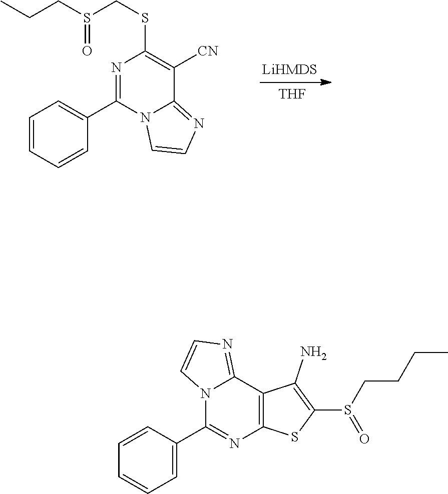 [SMILES: CCCS(=O)CSc1nc(-c2ccccc2)n2ccnc2c1C#N ; "THF" ; CCCCS(=O)c1sc2nc(-c3ccccc3)n3ccnc3c2c1N]

0.28 g of 7-(butylsulfinylmethylsulfanyl)-5-phenyl-imidazo[1,2-c]pyrimidine-8-carbonitrile was dissolved in 0.60 mL of tetrahydrofuran, which was then cooled to 0° C. 1.13 mL (with a concentration of 1.00 mol/L) of lithium bis(trimethylsilyl)amide was slowly added dropwise to the reaction solution, which was then replaced by nitrogen 3 times. After the addition was complete, stirring was continued at 0° C. for 15 minutes. The reaction was completed as detected by LCMS. After the reaction solution was restored to room temperature, 5 mL of water was added to the reaction solution, which was then extracted twice with ethyl acetate. The organic phase was concentrated to obtain the crude product. The obtained crude product was separated and purified using prep-HPLC to obtain the compound of Example 1.

MS (ESI) m/z=371.1 (M+H)⁺, ¹H NMR (400 MHz, DMSO-d₆) δ 8.03 (d, J=1.7 Hz, 1H), 7.99-7.96 (m, 2H), 7.72-7.64 (m, 4H), 6.54 (s, 2H), 3.21-3.15 (m, 1H), 3.08-3.01 (m, 1H), 1.66-1.53 (m, 2H), 1.49-1.40 (q, J=7.4 Hz, 2H), 0.92-0.88 (t, J=7.3 Hz, 3H).

### Example 2: Preparation of 8-(butylsulfinyl)-9-methoxy-5-phenylimidazo[1,2-c]thieno[3,2-e]pyrimidine

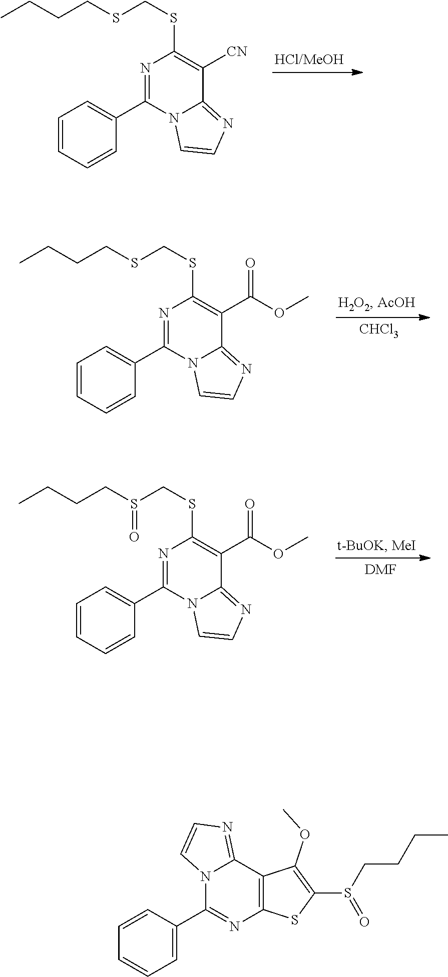

### Step 1: Preparation of methyl 7-(butylsulfanylmethylsulfanyl)-5-phenyl-imidazo[1,2-c]pyrimidine-8-carboxylate

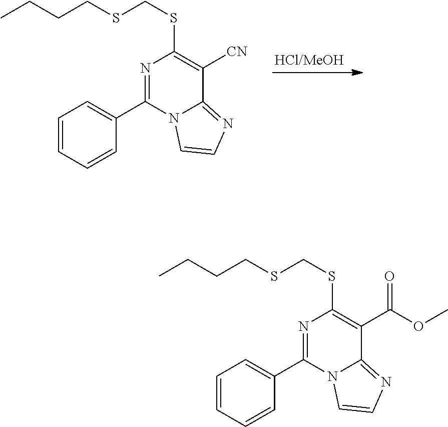 [SMILES: CCCCSCSc1nc(-c2ccccc2)n2ccnc2c1C#N ; CCCCSCSc1nc(-c2ccccc2)n2ccnc2c1C(=O)OC]

At room temperature, 0.24 g of 7-(butylsulfanylmethylsulfanyl)-5-phenyl-imidazo[1,2-c]pyrimidine-8-carbonitrile was added to 3.84 mL of methanolic hydrochloric acid solution (4 mol/L). The reaction solution was stirred at 85° C. for 15 hours. LCMS and TLC (petroleumether:ethyl acetate=1:1) showed that the reaction was completed. The reaction solution was concentrated under reduced pressure to obtain the crude product. The crude product was separated and purified using silica gel column chromatography to obtain the title compound. MS (ESI) m/z=388.2 (M+H)⁺.

### Step 2: Preparation of methyl 7-(butylsulfinylmethylsulfanyl)-5-phenyl-imidazo[1,2-c]pyrimidine-8-carboxylate

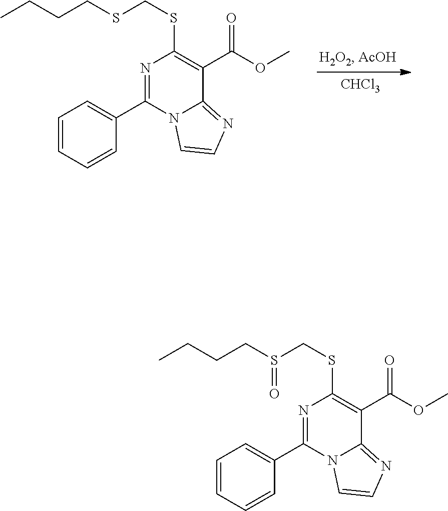

0.48 g of methyl 7-(butylsulfanylmethylsulfanyl)-5-phenyl-imidazo[1,2-c]pyrimidine-8-carboxylate was added into 2.3 mL of chloroform. At room temperature, 1.12 g of acetic acid and 351 mg of hydrogen peroxide (with a content of 30%) were added to the reaction solution, which was then stirred at room temperature for 13 hours. LCMS and TLC (petroleumether:ethyl acetate=0:1) showed that the reaction was completed. The reaction solution was poured into the aqueous solution of sodium sulfite, which was then extracted twice with dichloromethane and concentrated under reduced pressure to obtain the crude product. The crude product was separated and purified using prep-TLC (petroleumether:ethyl acetate=0:1) to obtain the title compound. MS (ESI) m/z=404.1 (M+H)⁺, ¹H NMR (400 MHz, CDCl₃-d) (8.02 (dd, J=1.6, 7.9 Hz, 2H), 7.86 (d, J=1.4 Hz, 1H), 7.77 (s, 1H), 7.73-7.64 (m, 3H), 4.77 (d, J=13.0 Hz, 1H), 4.26 (d, J=13.0 Hz, 1H), 4.16 (s, 3H), 2.97 (td, J=8.1, 13.0 Hz, 1H), 2.86-2.71 (m, 1H), 1.81 (quin, J=7.7 Hz, 2H), 1.52-1.41 (m, 2H), 0.95 (t, J=7.4 Hz, 3H).

### Step 3: Preparation of 8-(butylsulfinyl)-9-methoxy-5-phenylimidazo[1,2-c]thieno[3,2-e]pyrimidine

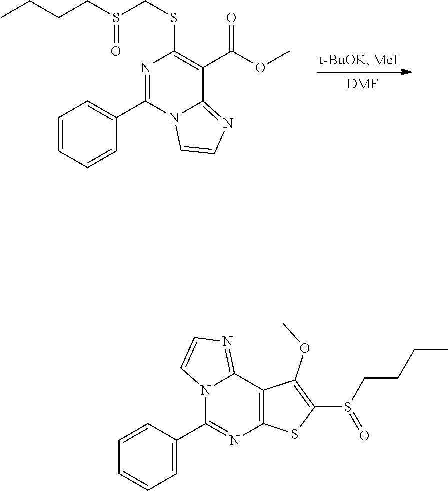

0.1 g of methyl 7-(butylsulfinylmethylsulfanyl)-5-phenyl-imidazo[1,2-c]pyrimidine-8-carboxylate was added into 1.0 mL of N,N-dimethylformamide. At 0° C., 41.71 mg of potassium tert-butoxide and 70.35 mg of iodomethane were added into the system under nitrogen protection, which was then stirred at room temperature for 2 hours. LCMS showed that the reaction was completed. The reaction solution was added into water to quench, and then extracted twice with ethyl acetate. The organic phase was dried and concentrated to obtain the crude product. The obtained crude product was separated and purified using Prep-HPLC to obtain the compound of Example 2. MS (ESI) m/z=386.0 (M+H)⁺, ¹H NMR (400 MHz, CDCl₃-d) (7.89-7.81 (m, 2H), 7.78 (s, 1H), 7.60 (s, 1H), 7.59-7.51 (m, 3H), 4.28 (s, 3H), 3.21-3.12 (m, 1H), 3.09-3.00 (m, 1H), 1.77-1.64 (m, 2H), 1.51-1.36 (m, 2H), 0.88 (t, J=7.3 Hz, 3H).

### Example 3: Preparation of 2-((9-amino-5-phenylimidazo[1,2-c]thieno[3,2-e]pyrimidin-8-yl)sulfinyl)ethan-1-ol

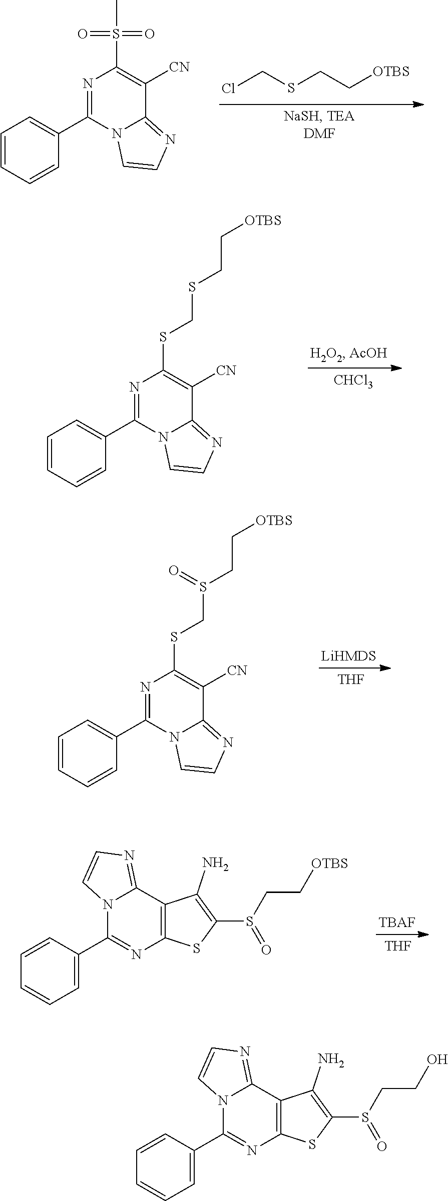

### Step 1: Preparation of 7-[2-[tert-butyl(dimethyl)silyl]oxyethylsulfanylmethylsulfanyl]-5-phenyl-imidazo[1,2-c]pyrimidine-8-carbonitrile

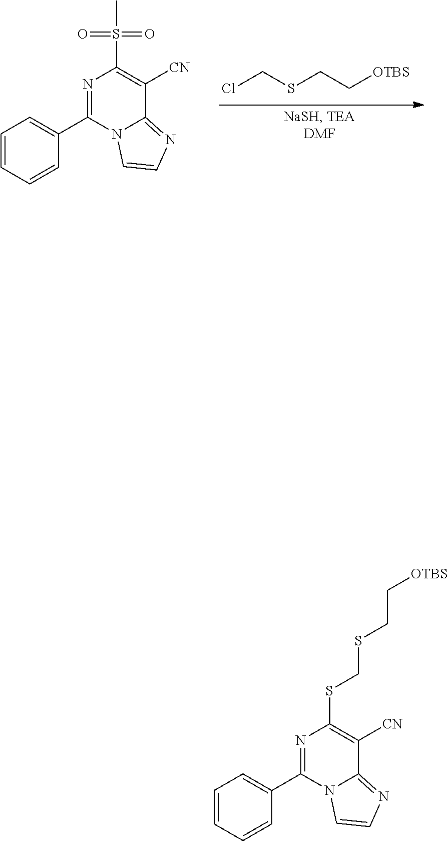 [SMILES: CS(=O)(=O)c1nc(-c2ccccc2)n2ccnc2c1C#N ; CC(C)(C)[Si](C)(C)OCCSCCl ; CC(C)(C)[Si](C)(C)OCCSCSc1nc(-c2ccccc2)n2ccnc2c1C#N]

At room temperature, 3.25 g of 7-methylsulfonyl-5-phenyl-imidazo[1,2-c]pyrimidine-8-carbonitrile was dissolved in 25 mL of N,N-dimethylformamide, into which 1.22 g of sodium hydrosulfide was added, which was then heated to 80° C. and reacted under a sealed condition for 20 minutes. TLC (petroleum ether:ethyl acetate=1:1) showed that the raw materials were completely depleted. The reaction system was cooled to room temperature, into which 3.31 g of triethylamine and 3.94 g of tert-butyl-[2-(chloromethylsulfanyl)ethoxy]-dimethylsilane were added, which was then heated to 80° C. and reacted under a sealed condition for 2 hours. The reaction was completed as detected by LCMS. The reaction system was cooled to room temperature, quenched by adding water, and extracted with ethyl acetate 3 times. The organic phases were combined, dried over anhydrous sodium sulfate, filtered, and concentrated under reduced pressure to obtain the crude product. The crude product was separated and purified by column chromatography (petroleumether:ethyl acetate=50:1 to 10:1) to obtain the title compound. MS (ESI) m/z=457.3 (M+H)⁺.

### Step 2: Preparation of 7-[2-[tert-butyl(dimethyl)silyl]oxyethylsulfinylmethylsulfanyl]-5-phenyl-imidazo[1,2-c]pyrimidine-8-carbonitrile

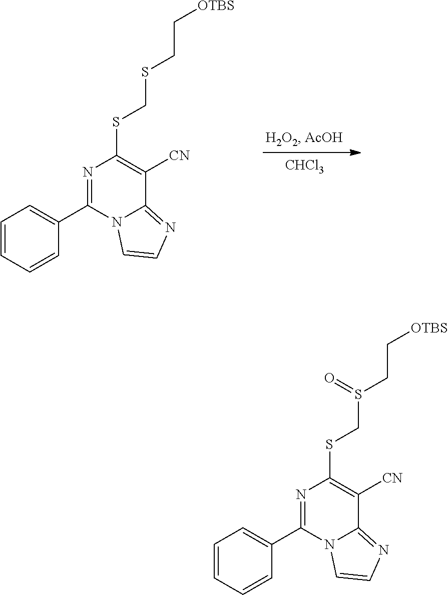 [SMILES: CC(C)(C)[Si](C)(C)OCCSCSc1nc(-c2ccccc2)n2ccnc2c1C#N ; "CHCl3" ; CC(C)(C)[Si](C)(C)OCCS(=O)CSc1nc(-c2ccccc2)n2ccnc2c1C#N]

At room temperature, 1.16 g of 7-[2-[tert-butyl(dimethyl)silyl]oxyethylsulfanylmethylsulfanyl]-5-phenyl-imidazo[1,2-c]pyrimidine-8-carbonitrile was dissolved in 23.2 mL of chloroform and 2.29 g of acetic acid, into which 719 mg of hydrogen peroxide was added dropwise, which was then heated to 40° C. and stirred for 2 hours. LCMS showed that the raw materials were completely reacted. The reaction liquid was added to 15.0 mL of saturated aqueous solution of sodium bicarbonate and 20.0 mL of saturated aqueous solution of sodium sulfite, which was extracted twice with dichloromethane. The organic phases were combined, dried over anhydrous sodium sulfate, filtered and concentrated to obtain the crude product. The crude product was purified by a preparative TLC (petroleumether:ethyl acetate=0:1) to obtain the title compound. MS (ESI) m/z=473.3 (M+H)⁺.

### Step 3: Preparation of 8-((2-((tert-butyldimethylsilyl)oxy)ethyl)sulfinyl)-5-phenylimidazo[1,2-c]thieno[3,2-e]pyrimidin-9-amine

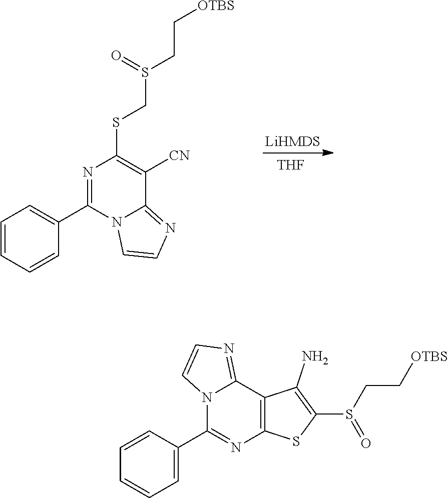 [SMILES: CC(C)(C)[Si](C)(C)OCCS(=O)CSc1nc(-c2ccccc2)n2ccnc2c1C#N ; CC(C)(C)[Si](C)(C)OCCS(=O)c1sc2nc(-c3ccccc3)n3ccnc3c2c1N ; "THF"]

At 0° C., 0.25 g of 7-[2-[tert-butyl(dimethyl)silyl]oxyethylsulfinylmethylsulfanyl]-5-phenyl-imidazo[1,2-c]pyrimidine-8-carbonitrile was dissolved in 5.0 mL of tetrahydrofuran, into which 652 μL of lithium hexamethyldisilazide (with a concentration of 1.0 mol/L) was added dropwise. The reaction solution was stirred at 0° C. for 20 minutes. LCMS showed that the reaction was completed. The reaction solution was added to water, which was then extracted twice with ethyl acetate. The organic phases were combined and concentrated under reduced pressure to obtain the crude product. The obtained crude product was purified using a preparative TLC (petroleum ether:ethyl acetate=0:1) to obtain the title compound. MS (ESI) m/z=473.5 (M+H)⁺, ¹H NMR (400 MHz, CDCl₃-d) δ 7.95-7.90 (m, 2H), 7.83 (d, J=1.6 Hz, 1H), 7.67-7.58 (m, 4H), 6.14-5.83 (m, 2H), 4.18-3.90 (m, 2H), 3.65-3.23 (m, 2H), 0.97-0.89 (m, 9H), 0.12 (d, J=8.3 Hz, 6H).

### Step 4: Preparation of 2-((9-amino-5-phenylimidazo[1,2-c]thieno[3,2-e]pyrimidin-8-yl)sulfinyl)ethan-1-ol

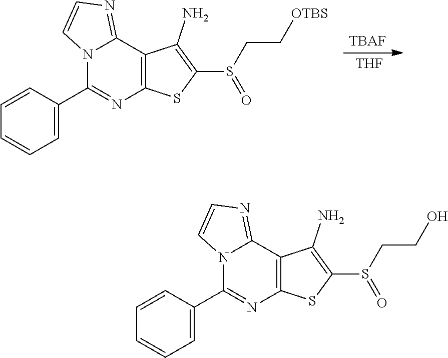

At room temperature, 80 mg of 8-((2-((tert-butyldimethylsilyl)oxy)ethyl)sulfinyl)-5-phenylimidazo[1,2-c]thieno[3,2-e]pyrimidin-9-amine was dissolved in 0.50 mL of tetrahydrofuran, into which 202 μL of tetrabutylammonium fluoride (with a concentration of 1.0 mol/L) was added. The reaction system was stirred at 25° C. for 2 hours. LCMS showed that the raw materials were completely depleted. The reaction solution was concentrated under reduced pressure to obtain the crude product. The crude product was separated and purified by a preparative HPLC to obtain the title compound of Example 3.

MS (ESI) m/z=358.9 (M+H)⁺, ¹H NMR (400 MHz, CDCl₃-d) δ=7.85 (dd, J=1.8, 7.5 Hz, 2H), 7.76 (d, J=1.4 Hz, 1H), 7.61-7.49 (m, 4H), 6.17-5.63 (m, 2H), 4.27-4.17 (m, 1H), 4.16-4.03 (m, 1H), 3.54 (ddd, J=3.4, 6.8, 13.4 Hz, 1H), 3.20 (ddd, J=3.4, 7.2, 13.3 Hz, 1H).

### Example 4: Preparation of 8-(butylsulfinyl)-3-methyl-5-phenylthieno[3,2-e][1,2,4]triazolo[4,3-c]pyrimidin-9-amine

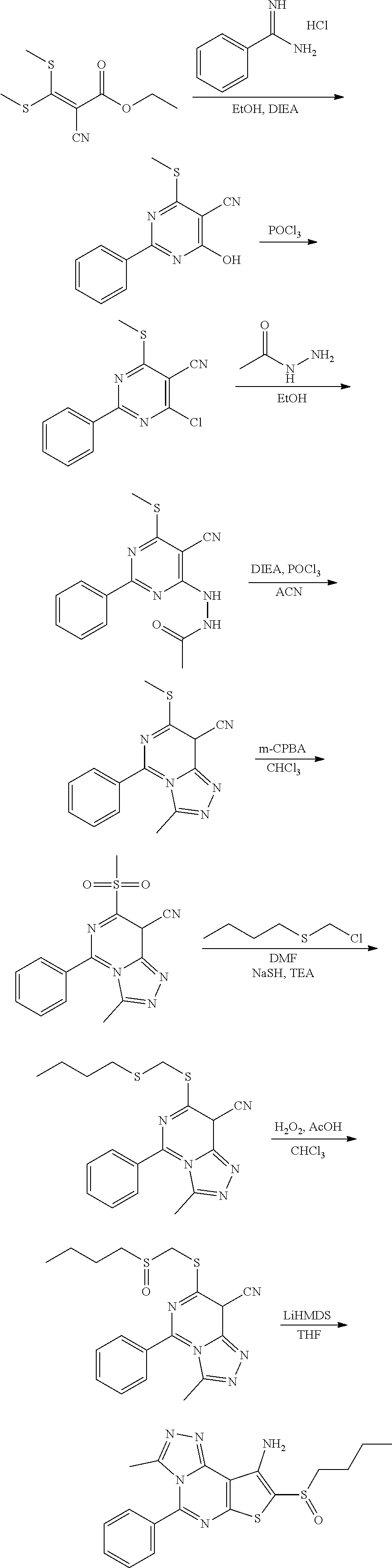

### Step 1: Preparation of 4-hydroxy-6-methylthio-2-phenyl-pyrimidine-5-carbonitrile

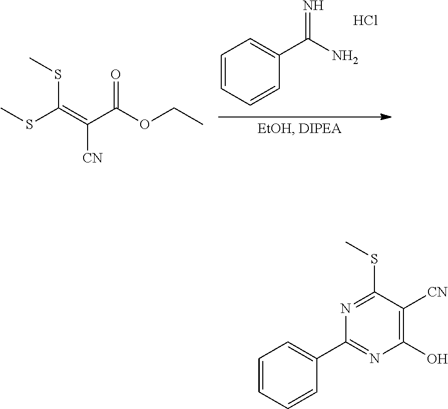

10.0 g of ethyl 2-cyano-3,3-bis(methylthio)prop-2-enoate and 7.93 g of benzamidine hydrochloride were added into a 500 mL 3-neck flask. At 20° C., 100 mL of ethanol and 9.62 mL of N,N-diisopropylethylamine were added into the reactor. After the addition was completed, the temperature was raised to 80° C., and stirred at this temperature for 12 hours. LCMS showed that the reaction was completed. 100 mL of water was added into the reaction solution, and stirring was continued at room temperature (20° C.) for 0.5 hours. The reaction solution was filtered under reduced pressure to obtain the title compound.

MS (ESI) m/z=244.1 (M+H)⁺, ¹H NMR (400 MHz, DMSO-d₆) (13.65-13.36 (m, 1H), 8.28-8.16 (m, 2H), 7.72-7.67 (m, 1H), 7.63-7.57 (m, 2H), 2.72 (s, 3H).

### Step 2: Preparation of 4-chloro-6-methylthio-2-phenyl-pyrimidine-5-carbonitrile

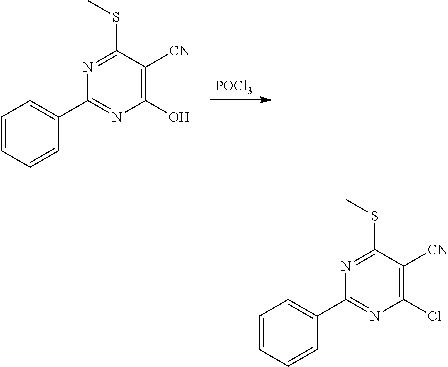

At room temperature (25° C.), 4.00 g of 4-hydroxy-6-methylthio-2-phenyl-pyrimidine-5-carbonitrile was added to 40 mL of phosphorus oxychloride. After the addition was completed, the temperature was raised to 80° C., and the reaction was carried out at this temperature for 12 hours. The raw materials were completely reacted as detected by LCMS. After the reaction solution was cooled to room temperature, the reaction solution was slowly added into water to quench, with stirring throughout the addition. An aqueous solution of sodium bicarbonate was added to adjust the reaction solution to alkaline. Subsequently, ethyl acetate was added to extract twice. The organic phase was concentrated to obtain the title compound.

MS (ESI) m/z 262.1=(M+H)⁺, ¹H NMR (400 MHz, DMSO-d₆) δ 8.48-8.36 (m, 2H), 7.42-7.37 (m, 1H), 7.63-7.57 (m, 2H), 2.72 (s, 3H).

### Step 3: Preparation of N-(5-cyano-6-methylsulfanyl-2-phenyl-pyrimidin-4-yl)acetohydrazide

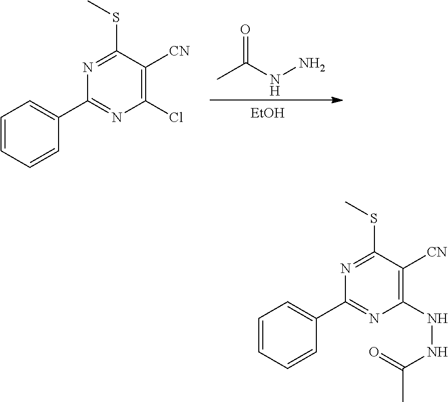

At room temperature (25° C.), 3.60 g of 4-chloro-6-methylthio-2-phenyl-pyrimidine-5-carbonitrile was added into 36 mL of ethanol. 1.53 g of acethydrazide was added into the reaction solution. After the addition was completed, the temperature was raised to 80° C., and the reaction was carried out at this temperature for 12 hours. The disappearance of raw materials was detected by LCMS. After the reaction solution was restored to room temperature, 60 mL of water were added into the reaction solution, which was subjected to liquid-liquid extraction with 30 mL of ethyl acetate. The organic phase was concentrated to obtain the crude product. The crude product was slurred and purified by adding 5 mL of ethyl acetate and 30 mL of tert-butyl methyl ether to obtain the title compound. MS(ESI) m/z=300.2 (M+H)⁺, ¹H NMR (400 MHz, DMSO-d₆) δ 10.44-9.90 (m, 2H), 8.38 (br d, J=7.1 Hz, 2H), 7.62-7.52 (m, 3H), 2.71 (s, 3H), 1.99 (s, 3H).

### Step 4: Preparation of 3-methyl-7-(methylthio)-5-phenyl-[1,2,4]triazolo[4,3-c]pyrimidine-8-carbonitrile

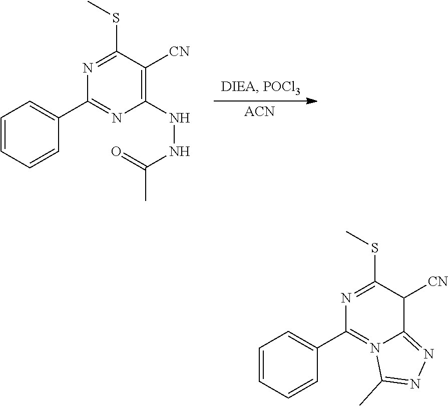

At room temperature (25° C.), 1.38 g of N-(5-cyano-6-methylsulfanyl-2-phenyl-pyrimidin-4-yl)acetohydrazide was added into 13.8 mL of acetonitrile. Subsequently, 642 μL of N,N-diisopropylethylamine and 214 μL of phosphorus oxychloride were slowly added dropwise into the reaction solution. After the addition was completed, the temperature was raised to 90° C., and the reaction was carried out at this temperature for 12 hours. The raw materials were completely reacted as detected by TLC and LCMS. After the reaction solution was cooled to room temperature, the reaction solution was slowly added into water to quench, with stirring throughout the addition. An aqueous solution of sodium bicarbonate was added to adjust the reaction solution to alkaline. Subsequently, ethyl acetate was added to extract twice. The organic phase was concentrated. The obtained crude product was separated and purified by column chromatography (petroleumether:ethyl acetate=50:1 to 0:1) to obtain the title compound.

MS (ESI) m/z=282.1 (M+H)⁺, ¹H NMR (400 MHz, CDCl₃-d) δ 8.78-8.72 (m, 2H), 7.75-7.61 (m, 3H), 2.85 (s, 3H), 2.70 (s, 3H).

### Step 5: Preparation of 3-methyl-7-(methylsulfonyl)-5-phenyl-[1,2,4]triazolo[4,3-c]pyrimidine-8-carbonitrile

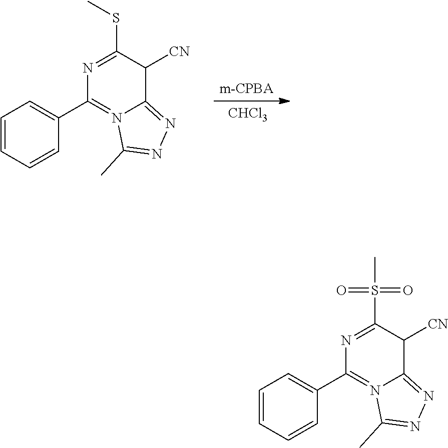

At room temperature (25° C.), 0.6 g of 3-methyl-7-(methylthio)-5-phenyl-[1,2,4]triazolo[4,3-c]pyrimidine-8-carbonitrile was dissolved in 6 mL of trichloromethane. 1.15 g of m-chloroperoxybenzoic acid was slowly added into the reaction solution under nitrogen protection. After the addition was complete, the reaction was carried out at room temperature (25° C.) for 3 hours. The reaction was completed as detected by LCMS. The reaction solution was added into 20 mL of saturated aqueous solution of sodium bicarbonate, and then extracted twice with dichloromethane. The organic phases were combined and concentrated, and then quenched with aqueous solution of sodium sulfite to obtain the title compound.

MS (ESI) m/z=314.1 (M+H)⁺.

### Step 6: Preparation of 7-(((butylthio)methyl)thio)-3-methyl-5-phenyl-[1,2,4]triazolo[4,3-c]pyrimidine-8-carbonitrile

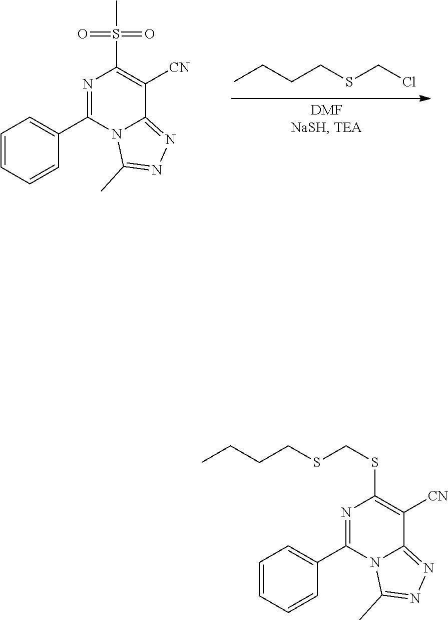

At room temperature (25° C.), 1.00 g of 3-methyl-7-(methylsulfonyl)-5-phenyl-[1,2,4]triazolo[4,3-c]pyrimidine-8-carbonitrile was added into 10 mL of N,N-dimethylformamide. Subsequently, 250 mg of sodium hydrosulfide was added into the reaction solution, which was then heated to 80° C. and reacted at this temperature for 20 minutes. After the reaction was completed as detected by TLC, it was cooled to room temperature. Subsequently, 1.33 mL of triethylamine and 885 mg of 1-(chloromethylthio)butane were added to the reaction solution. After the addition was completed, the temperature was raised to 80° C., and the reaction was carried out at this temperature for 2 hours. The reaction was completed as detected by TLC and LCMS. After the reaction solution was cooled to room temperature, water (30 mL) was added to the reaction solution, which was then extracted twice with ethyl acetate. The organic phase was concentrated to obtain the crude product. The obtained crude product was separated and purified by column chromatography (petroleumether:ethyl acetate=10:1 to 0:1) to obtain the title compound. MS (ESI) m/z=370.4 (M+H)⁺, ¹H NMR (400 MHz, CDCl₃-d) δ 8.80-8.68 (m, 2H), 7.75-7.61 (m, 3H), 4.60 (s, 2H), 2.82-2.73 (m, 2H), 2.72-2.63 (m, 3H), 1.65 (br d, J=7.3 Hz, 2H), 1.52-1.37 (m, 2H), 1.04-0.89 (m, 3H).

### Step 7: Preparation of 7-(((butylsulfinyl)methyl)thio)-3-methyl-5-phenyl-[1,2,4]triazolo[4,3-c]pyrimidine-8-carbonitrile

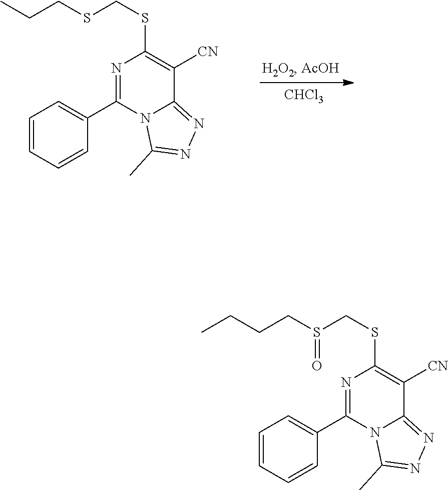 [SMILES: CCCSCSc1nc(-c2ccccc2)n2c(C)nnc2c1C#N ; "CHCl3" ; CCCCS(=O)CSc1nc(-c2ccccc2)n2c(C)nnc2c1C#N]

At room temperature (25° C.), 0.52 g of 7-(((butylthio)methyl)thio)-3-methyl-5-phenyl-[1,2,4]triazolo[4,3-c]pyrimidine-8-carbonitrile was added to 5.2 mL of trichloromethane. Subsequently, 1.21 mL of glacial acetic acid and 338 μL of hydrogen peroxide were sequentially added to the reaction solution. After the addition was completed, the temperature was raised to 40° C., and the reaction was carried out at this temperature for 3 hours. The reaction was completed as detected by LCMS and TLC. After the reaction solution was cooled to room temperature, a saturated aqueous solution of sodium bicarbonate (15 mL) was added to the reaction solution, which was then extracted twice with dichloromethane. The organic phase was concentrated to obtain the crude product. The crude product was purified by prep-TLC to obtain the title compound.

MS (ESI) m/z 386.3=(M+H)⁺. ¹H NMR (400 MHz, CDCl₃-d) δ 8.66-8.60 (m, 2H), 7.67-7.53 (m, 3H), 4.75-4.39 (m, 2H), 2.94-2.71 (m, 2H), 2.62 (s, 3H), 1.75 (quin, J=7.6 Hz, 2H), 1.46-1.37 (m, 2H), 0.89 (t, J=7.3 Hz, 3H).

### Step 8: Preparation of 8-(butylsulfinyl)-3-methyl-5-phenylthieno[3,2-e][1,2,4]triazolo[4,3-c]pyrimidin-9-amine

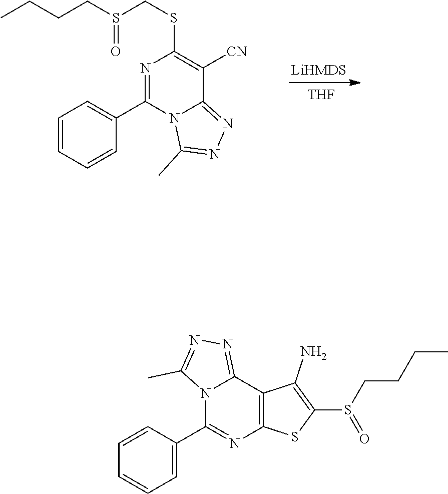 [SMILES: CCCCS(=O)CSc1nc(-c2ccccc2)n2c(C)nnc2c1C#N ; "THF" ; CCCCS(=O)c1sc2nc(-c3ccccc3)n3c(C)nnc3c2c1N]

0.2 g of 7-(((butylsulfinyl)methyl)thio)-3-methyl-5-phenyl-[1,2,4]triazolo[4,3-c]pyrimidine-8-carbonitrile was dissolved in 2.0 mL of tetrahydrofuran, which was then cooled to 0° C. At this temperature, 78 μL of the tetrahydrofuran solution of lithium bis(trimethylsilyl)amide was slowly added dropwise to the reaction solution, which was then replaced by nitrogen 3 times. After the addition was complete, stirring was continued at 0° C. for 2 hours. The reaction was completed as detected by LCMS. After the reaction solution was restored to room temperature, water (10 mL) was added to the reaction solution, which was then extracted twice with ethyl acetate. The organic phase was concentrated to obtain the crude product. The crude product was purified by prep-HPLC (column: Waters xbridge 150*25 mm 0 m; mobile phase: [water (NH₄HCO₃)-ACN]; B %: 32%-˜62%, 10 min) to obtain the title compound.

MS(ESI) m/z 386.2=(M+H)⁺, ¹H(H NMR (400 MHz, CDCl₃-d)₃ 8.67-8.54 (m, 2H), 7.68-7.56 (m, 3H), 6.12-5.38 (m, 2H), 3.43-3.09 (m, 2H), 2.72 (s, 3H), 1.89-1.78 (m, 2H), 1.58-1.51 (m, 2H), 1.04-0.93 (in, 3H).

A series of compounds were prepared from corresponding commercial reagents and the products in the foregoing preparation Examples and Examples as raw materials, by using preparation methods similar to those of the foregoing Examples, and structures and characterization data of the compounds are shown in Table 1:

TABLE 1

| | | Preparation | |
|---|---|---|---|
| | | method/ | |
| | | reference | |
| Example | Structure | example | MS(M + H)⁺ and ¹H NMR |
| 5 | 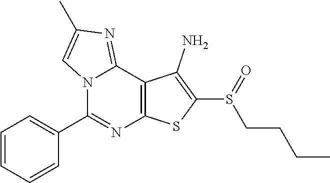 [SMILES: CCCCS(=O)c1sc2nc(-c3ccccc3)n3cc(C)nc3c2c1N] | Method 1-1/ Example 1 | MS (ESI) m/z = 385.0 (M + H)⁺, ¹H NMR (400 MHz, CDCl₃-d) δ 7.92 (dd, J = 1.9, 7.4 Hz, 2H), 7.67-7.61 (m, 3H), 7.58 (s, 1H), 6.15-5.85 (m, 2H), 3.46-3.08 (m, 2H), 2.52 (br s, 3H), 1.86-1.72 (m, 2H), 1.51-1.40 (m, 2H), 0.98 (t, J = 7.3 Hz, 3H) |
| 6 | 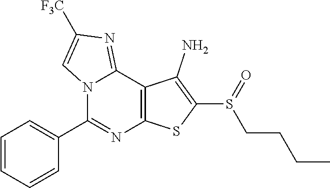 [SMILES: CCCCS(=O)c1sc2nc(-c3ccccc3)n3cc(C(F)(F)F)nc3c2c1N] | Method 1-1/ Example 1 | MS (ESI) m/z = 439.0 (M + H)⁺, ¹H NMR (400 MHz, CDCl₃-d) δ 8.03 (s, 1H), 7.82 (dd, J = 1.6, 7.7 Hz, 2H), 7.62-7.54 (m, 3H), 5.82 (s, 2H), 3.26 (ddd, J = 5.9, 9.2, 12.7 Hz, 1H), 3.10 (ddd, J = 6.3, 9.2, 12.8 Hz, 1H), 1.77-1.56 (m, 2H), 1.44 (dq, J = 2.1, 7.4 Hz, 2H), 0.89 (t, J = 7.4 Hz, 3H) |
| 7 | 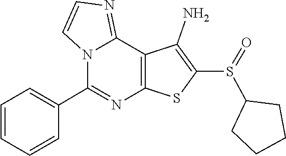 [SMILES: Nc1c(S(=O)C2CCCC2)sc2nc(-c3ccccc3)n3ccnc3c12] | Method 1-1/ Example 1 | MS (ESI) m/z = 383.1 (M + H)⁺, ¹H NMR (400 MHz, DMSO-d₆) δ 8.02 (d, J = 1.6 Hz, 1H), 8.00-7.95 (m, 2H), 7.69 (q, J = 2.3 Hz, 2H), 7.68 (s, 2H), 6.58-6.50 (m, 2H), 3.62- 3.58 (m, 1H), 2.14-2.06 (m, 1H), 2.00-1.91 (m, 1H), 1.74 (br s, 2H), 1.68-1.64 (m, 2H), 1.63-1.55 (m, 2H) |
| 8 | 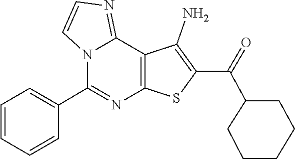 [SMILES: Nc1c(C(=O)C2CCCCC2)sc2nc(-c3ccccc3)n3ccnc3c12] | Method 1-1/ Example 1 | MS (ESI) m/z = 397.1 (M + H)⁺, ¹H NMR (400 MHz, DMSO-d₆) δ 8.02 (d, J = 1.6 Hz, 1H), 7.99-7.95 (m, 2H), 7.72-7.64 (m, 4H), 6.49 (s, 2H), 3.07-2.97 (m, 1H), 2.19- 2.09 (m, 1H), 1.84 (br d, J = 12.9 Hz, 1H), 1.79- 1.67 (m, 2H), 1.63 (br d, J = 10.4 Hz, 1H), 1.53- 1.42 (m, 1H), 1.42-1.30 (m, 2H), 1.30-1.16 (m, 2H) |
| 9 | 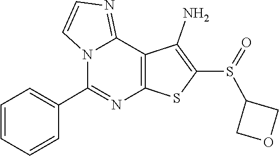 [SMILES: Nc1c(S(=O)C2COC2)sc2nc(-c3ccccc3)n3ccnc3c12] | Method 1-1/ Example 1 | MS (ESI) m/z = 371.0 (M + H)⁺, ¹H NMR (400 MHz, CDCl₃-d) δ 7.96-7.88 (m, 2H), 7.83 (d, J = 0.8 Hz, 1H), 7.73-7.58 (m, 4H), 6.02 (br s, 2H), 5.30-5.22 (m, 1H), 5.13- 5.03 (m, 1H), 4.89 (t, J = 7.8 Hz, 1H), 4.79- 4.72 (m, 1H), 4.67-4.57 (m, 1H) |
| 10 | 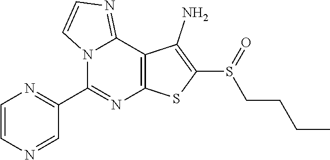 [SMILES: CCCCS(=O)c1sc2nc(-c3cnccn3)n3ccnc3c2c1N] | Method 1-1/ Example 1 | MS (ESI) m/z = 373.1 (M + H)⁺, ¹H NMR (400 MHz, DMSO-d₆) δ 9.67 (d, J = 1.4 Hz, 1H), 9.14 (d, J = 1.7 Hz, 1H), 8.95-8.93 (m, 2H), 7.82 (d, J = 1.6 Hz, 1H), 6.61 (s, 2H), 3.22-3.16 (m, 1H), 3.12-3.05 (m, 1H), 1.67-1.58 (m, 2H), 1.50-1.41 (m, 2H), 0.92-0.89(t, J = 7.3 Hz, 3H) |
| 11 | 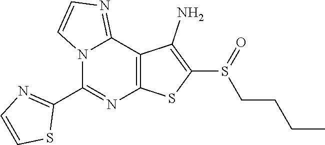 [SMILES: CCCCS(=O)c1sc2nc(-c3nccs3)n3ccnc3c2c1N] | Method 1-1/ Example 1 | MS (ESI) m/z = 378.1 (M + H)⁺, ¹H NMR (400 MHz, DMSO-d₆) δ 9.33 (d, J = 1.6 Hz, 1H), 8.31 (d, J = 3.1 Hz, 1H), 8.26 (d, J = 3.2 Hz, 1H), 7.85 (d, J = 1.6 Hz, 1H), 6.59 (s, 2H), 3.21-3.15 (m, 1H), 3.11-3.05 (m, 1H), 1.67-1.57 (dt, J = 14.7, 7.5 Hz, 2H), 1.50-1.42 (m, 2H), 0.92-0.89 (t, J = 7.3 Hz, 3H) |
| 12 | 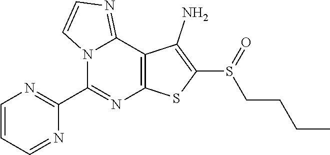 [SMILES: CCCCS(=O)c1sc2nc(-c3ncccn3)n3ccnc3c2c1N] | Method 1-1 Example 1 | MS (ESI) m/z = 373.1 (M + H)⁺, ¹H NMR (400 MHz, DMSO-d₆) δ 9.17 (d, J = 4.9 Hz, 2H), 8.90 (d, J = 1.8 Hz, 1H), 7.84-7.74 (m, 2H), 6.59 (s, 2H), 3.19 (ddd, J = 12.7, 8.9, 5.8 Hz, 1H), 3.08 (ddd, J = 12.7, 8.9, 6.3 Hz, 1H), 1.63 (h, J = 6.9 Hz, 2H), 1.45 (q, J = 7.4 Hz, 2H), 0.90 (t, J = 7.2 Hz, 3H) |
| 13 | 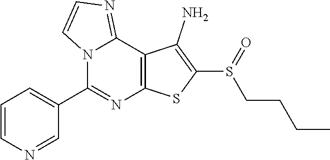 [SMILES: CCCCS(=O)c1sc2nc(-c3cccnc3)n3ccnc3c2c1N] | Method 1-1/ Example 1 | MS (ESI) m/z = 372.1 (M + H)⁺, ¹H NMR (400 MHz, CDCl₃-d) 8 9.31-9.18 (m, 1H), 8.90 (br d, J = 3.4 Hz, 1H), 8.29 (br d, J = 8.0 Hz, 1H), 7.82 (s, 1H), 7.68 (d, J = 0.9 Hz, 1H), 7.60 (br dd, J = 4.9, 7.7 Hz, 1H), 6.09-5.82 (m, 2H), 3.45-3.14 (m, 2H), 1.82-1.72 (m, 2H), 1.56-1.49 (m, 2H), 0.99 (t, J = 7.3 Hz, 3H) |
| 14 | 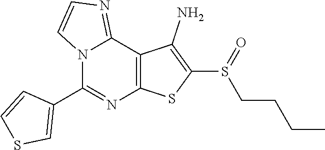 [SMILES: CCCCS(=O)c1sc2nc(-c3ccsc3)n3ccnc3c2c1N] | Method 1-1/ Example 1 | MS (ESI) m/z = 377.3 (M + H)⁺, ¹H NMR (400 MHz, CDCl₃-d) δ 8.19-8.12 (m, 1H), 7.99 (d, J = 1.1 Hz, 1H), 7.75 (d, J = 5.1 Hz, 1H), 7.67 (d, J = 1.4 Hz, 1H), 7.60 (dd, J = 2.9, 5.1 Hz, 1H), 5.92 (s, 2H), 3.39-3.17 (m, 2H), 1.82-1.70 (m, 2H), 1.55-1.49 (m, 2H), 0.98 (t, J = 7.3 Hz, 3H) |
| 15 | 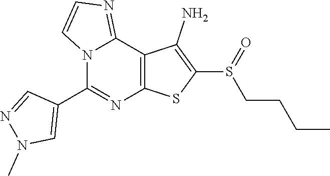 [SMILES: CCCCS(=O)c1sc2nc(-c3cnn(C)c3)n3ccnc3c2c1N] | Method 1-1/ Example 1 | MS (ESI) m/z = 375.3 (M + H)⁺, ¹H NMR (400 MHz, CDCl₃-d) δ 8.22 (d, J = 1.4 Hz, 2H), 7.96 (d, J = 1.6 Hz, 1H), 7.69 (d, J = 1.6 Hz, 1H), 5.90 (br s, 2H), 4.09 (s, 3H), 3.34 (ddd, J = 5.9, 9.2, 12.7 Hz, 1H), 3.18 (ddd, J = 6.3, 9.3, 12.8 Hz, 1H), 1.81-1.70 (m, 2H), 1.55 − 1.48 (m, 2H), 0.97 (t, J = 7.3 Hz, 3H) |
| 16 | 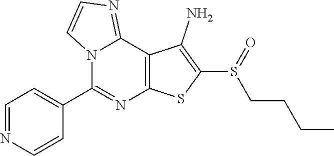 [SMILES: CCCCS(=O)c1sc2nc(-c3ccncc3)n3ccnc3c2c1N] | Method 1-1/ Example 1 | MS (ESI) m/z = 372.1 (M + H)⁺, ¹H NMR (400 MHz, CDCl₃-d) δ 8.86 (d, J = 6.0 Hz, 2H), 7.78 (d, J = 6.0 Hz, 2H), 7.74 (d, J = 1.4 Hz, 1H), 7.60 (d, J = 1.4 Hz, 1H), 5.88 (br s, 2H), 3.27 (ddd, J = 6.0, 9.2, 12.9 Hz, 1H), 3.11 (ddd, J = 6.4, 9.2, 12.7 Hz, 1H), 1.72-1.64 (m, 2H), 1.45 (br dd, J = 2.1, 7.4 Hz, 2H), 0.89 (t, J = 7.3 Hz, 3H) |
| 17 | 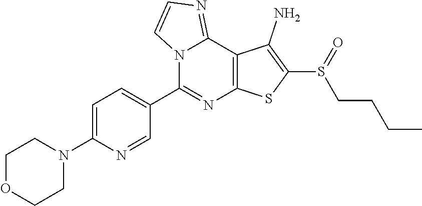 [SMILES: CCCCS(=O)c1sc2nc(-c3ccc(N4CCOCC4)nc3)n3ccnc3c2c1N] | Method 1-1/ Example 1 | MS (ESI) m/z = 457.1 (M + H)⁺, ¹H NMR (400 MHz, CDCl₃-d) δ 8.75 (d, J = 2.3 Hz, 1H), 8.01 (dd, J = 2.5, 9.0 Hz, 1H), 7.80 (d, J= 1.5 Hz, 1H), 7.54 (d, J = 1.5 Hz, 1H), 6.71 (d, J = 9.0 Hz, 1H), 5.82 (br s, 2H), 3.80-3.76 (m, 4H), 3.67-3.60 (m, 4H), 3.25 (ddd, J = 5.9, 9.2, 12.7 Hz, 1H), 3.09 (ddd, J = 6.3, 9.3, 12.7 Hz, 1H), 1.66-1.60 (m, 2H), 1.46-1.39 (m, 2H), 0.88 (t, J = 7.3 Hz, 3H) |
| 18 | 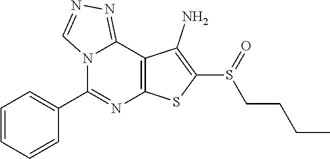 [SMILES: CCCCS(=O)c1sc2nc(-c3ccccc3)n3cnnc3c2c1N] | Method 2-1/ Example 4 | MS (ESI) m/z = 372.1 (M + H)⁺, ¹H NMR (400 MHz, DMSO-d₆) δ 8.82 (s, 1H), 8.50-8.48 (m, 2H), 7.72-7.63 (m, 3H), 6.44 (s, 2H), 3.23-3.16 (m, 1H), 3.11-3.04 (m, 1H), 1.66-1.57 (m, 2H), 1.50-1.40 (m, 2H), 0.92-0.88 (t, J = 7.3 Hz, 3H) |
| 19 | 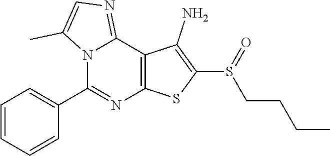 [SMILES: CCCCS(=O)c1sc2nc(-c3ccccc3)n3c(C)cnc3c2c1N] | Method 1-1/ Example 1 | MS (ESI) m/z =385.0 (M + H)⁺, ¹H NMR (400 MHz, CDCl₃-d) δ 7.86-7.80 (m, 2H), 7.58-7.50 (m, 3H), 7.47 (d, J = 0.9 Hz, 1H), 5.82 (s, 2H), 3.25 (ddd, J = 6.0, 9.2, 12.8 Hz, 1H), 3.15-3.02 (m, 1H), 2.39 (d, J = 0.8 Hz, 3H), 1.78-1.58 (m, 2H), 1.46-1.39 (m, 2H), 0.89 (t, J = 7.4 Hz, 3H) |
| 20 | 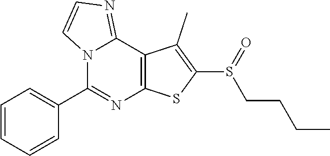 [SMILES: CCCCS(=O)c1sc2nc(-c3ccccc3)n3ccnc3c2c1C] | Method 1-2 Example 2 | MS (ESI) m/z = 370.0 (M + H)⁺, ¹H NMR (400 MHz, CDCl₃-d) δ 7.90-7.82 (m, 2H), 7.78 (d, J = 1.5 Hz, 1H), 7.62-7.49 (m, 4H), 3.23-3.13 (m, 1H), 2.98 (ddd, J = 6.7, 9.1, 12.8 Hz, 1H), 2.90 (s, 3H), 1.66 (tdd, J = 6.7, 8.5, 15.2 Hz, 2H), 1.49-1.37 (m, 2H), 0.89 (t, J = 7.3 Hz, 3H) |
| 21 | 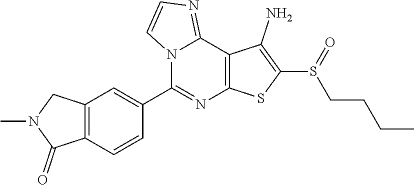 [SMILES: CCCCS(=O)c1sc2nc(-c3ccc4c(c3)CN(C)C4=O)n3ccnc3c2c1N] | Method 1-1/ Example 1 | MS (ESI) m/z = 440.1 (M + H)⁺, ¹H NMR (400 MHz, CDCl₃-d) δ = 8.01-7.92 (m, 3H), 7.73 (d, J = 1.6 Hz, 1H), 7.57 (d, J = 1.6 Hz, 1H), 5.86 (br s, 2H), 4.46 (s, 2H), 3.32- 3.23 (m, 1H), 3.21 (s, 3H), 3.14-3.05 (m, 1H), 1.73-1.59 (m, 2H), 1.45-1.39 (m, 2H), 0.89 (t, J = 7.3 Hz, 3H) |
| 22 | 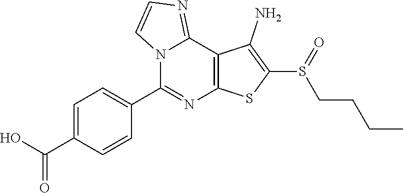 [SMILES: CCCCS(=O)c1sc2nc(-c3ccc(C(=O)O)cc3)n3ccnc3c2c1N] | Method 1-1/ Example 1 | MS (ESI) m/z = 415.1 (M + H)⁺, ¹H NMR (400 MHz, DMSO-d₆) δ 8.18-8.16 (d, J = 8.2 Hz, 2H), 8.09-8.07 (m, 3H), 7.71 (s, 1H), 6.54 (s, 2H), 3.21-3.14 (m, 1H), 3.08-3.01 (m, 1H), 1.65-1.55 (m, 2H), 1.49-1.40 (m, 2H), 0.92-0.88 (t, J = 7.3 Hz, 3H) |
| 23 | 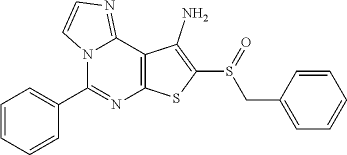 [SMILES: Nc1c(S(=O)Cc2ccccc2)sc2nc(-c3ccccc3)n3ccnc3c12] | Method 1-1/ Example 1 | MS (ESI) m/z = 404.9 (M + H)⁺, ¹H NMR (400 MHz, CDCl₃-d) δ = 7.88-7.82 (m, 2H), 7.75 (d, J = 1.5 Hz, 1H), 7.58-7.50 (m, 4H), 7.25-7.20 (m, 3H), 7.16-7.12 (m, 2H), 5.60 (s, 2H), 4.48-4.37 (m, 2H) |
| 24 | 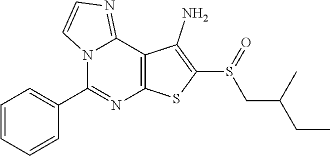 [SMILES: CCC(C)CS(=O)c1sc2nc(-c3ccccc3)n3ccnc3c2c1N] | Method 1-1/ Example 1 | MS (ESI) m/z = 385.2 (M + H)⁺, ¹H NMR (400 MHz, CDCl₃-d) δ = 7.89-7.82 (m, 2H), 7.75 (d, J = 1.1 Hz, 1H), 7.55 (dd, J = 1.3, 8.9 Hz, 4H), 5.83 (br d, J = 2.1 Hz, 2H), 3.46-2.70 (m, 2H), 2.02-1.72 (m, 1H), 1.49- 1.44 (m, 1H), 1.39-1.22 (m, 1H), 1.05 (dd, J = 3.9, 6.7 Hz, 3H), 0.87 (td, J = 7.4, 17.9 Hz, 3H) |
| 25 | 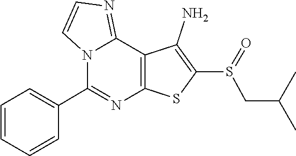 [SMILES: CC(C)CS(=O)c1sc2nc(-c3ccccc3)n3ccnc3c2c1N] | Method 1-1/ Example 1 | MS (ESI) m/z = 371.1 (M + H)⁺, ¹H NMR (400 MHz, CDCl₃-d) δ 7.88-7.82 (m, 2H), 7.75 (d, J = 1.5 Hz, 1H), 7.55 (dd, J = 1.1, 9.2 Hz, 4H), 5.83 (br s, 2H), 3.28 (dd, J = 6.4, 12.7 Hz, 1H), 2.87 (dd, J = 7.8, 12.7 Hz, 1H), 2.09 (td, J = 6.8, 13.9 Hz, 1H), 1.06 (dd, J = 1.7, 6.7 Hz, 6H) |
| 26 | 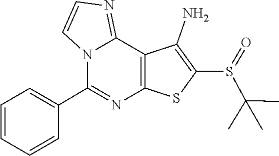 [SMILES: CC(C)(C)S(=O)c1sc2nc(-c3ccccc3)n3ccnc3c2c1N] | Method 1-1/ Example 1 | MS (ESI) m/z = 371.2 (M + H)⁺, ¹H NMR (400 MHz, CDCl₃-d) δ 7.92 (dd, J = 2.1, 7.4 Hz, 2H), 7.82 (d, J = 1.5 Hz, 1H), 7.66- 7.57 (m, 4H), 6.06 (s, 2H), 1.43 (s, 9H) |

### Biological Tests

### Test Example 1: Detection of Activity of 15-PGDH Kinase

### 1. Experimental Materials:

| Reagents/Materials/Instruments | Manufacturer | Item No./Model No. |
|---|---|---|
| 15-PGDH | Sino Biological Inc. | 11205-H08E |
| β-NAD | Sigma-Aldrich Corporation | N6522 |
| PGF2α | MedChemExpress LLC | HY-12956A |
| DMSO | Sigma-Aldrich Corporation | D8418 |
| 384-well plate | Corning United States | 4513 |
| | Corporation | |
| Tween 20 | Shanghai Macklin Biochemical | T818927 |
| | Technology Co., Ltd. | |
| Tris-HCl | Shanghai Beyotime Biotech Inc. | ST774 |
| Multifunctional | BMG LABTECH Corporation | PHERAstar ® FSX |
| microplate reader | | |

### 2. Experimental Method:

a. A solution of pH 7.5 containing 50 mM Tris-HCl, 0.01% Tween 20 was prepared with ultrapure water as a reaction buffer;
b. A 10 mM mother liquor of the compound to be tested was prepared with DMSO, and then the reaction buffer was used to dilute the mother liquor of the compound to be tested to obtain solution 1 of the compound to be tested at a concentration of 4000 nM, and then the solution 1 of the compound to be tested was serially diluted into solutions 2-10 of the compound to be tested at 9 concentrations with a gradient difference of three-fold. 5 μL of each of solutions 1-10 of the compound to be tested was taken and added into a 384-well plate as test wells;
c. 5 μL of the reaction buffer was added to the 384-well plate as positive control and blank control wells;
d. The reaction buffer was used to prepare a 15-PGDH protein solution at a concentration of 5 ng/μL, 5 μL of the 15-PGDH protein solution was taken and added to the test wells and positive control wells, and 5 μL of the reaction buffer was added to the blank control wells at the same time, then the plate was centrifuged at 2000 rpm for 30 seconds;
e. The reaction buffer was used to prepare 5 mM R-NAD and 2 mM PGF2α, respectively, which were mixed at 1:1 by volume to obtain a substrate mixture, 10 μL of the substrate mixture was taken and added to the test wells, positive control wells and blank control wells to start the reaction;
f. The fluorescence signal value (Ex/Em=340/450) of each well was detected continuously by using a multifunctional microplate reader.

### 3. Data Analysis:

a) Continuous fluorescence signal values were analyzed by using the “kinetic calculations-slope calculation method” in the PHERAstar Data analysis software to obtain the slope of each test well;
b) The inhibition rate % was calculated by using the following formula:

$$inhibition rate \% = [ 1 - ( slope of test well - signal value of positive control well ) / ( signal value of blank control well - average signal value of positive control well ) ] \times 100 \% .$$

c) Calculation of IC50 and plotting of inhibition rate-dose curves: IC50 values were calculated by fitting compound concentrations and corresponding inhibition rates with nonlinear regression (dose response-variable slope) via using GraphPad Prism 6.0. The formula is shown below:

$$Y = Bottom + ( Top - Bottom ) / ( 1 + 10 \hat{} ( ( Log IC 50 - X ) * HillSlope ) ) ,$$

wherein X is a log value of a concentration of the compound, and Y is inhibition rate %.

### 4. Experimental Results:

Inhibitory activities of some compounds in the present application against the 15-PGDH enzyme are as follows:

| Example No. | IC₅₀ (nM) |
|---|---|
| Example 1 | B |
| Example 4 | B |
| Example 5 | A+ |
| Example 6 | B |
| Example 7 | B |
| Example 8 | B |
| Example 9 | C |
| Example 10 | B |
| Example 11 | A |
| Example 12 | A |
| Example 13 | A+ |
| Example 14 | A |
| Example 15 | A |
| Example 16 | A |
| Example 17 | B |
| Example 18 | C |
| Example 19 | A |
| Example 20 | D |
| Example 21 | A |
| Example 22 | A |
| Example 23 | C |
| Example 24 | B |
| Example 25 | E |
| Example 26 | C |

In the table, “A+” represents that IC₅₀ of the inhibitory activity against 15-PGDH enzyme is in a range of less than 1.5 nM; “A” represents that IC₅₀ of the inhibitory activity against 15-PGDH enzyme is in a range of equal to or greater than 1.5 nM and less than 4 nM; “B” represents that IC₅₀ of the inhibitory activity against 15-PGDH enzyme is in a range of equal to or greater than 4 nM and less than 10 nM; “C” represents that IC₅₀ of the inhibitory activity against 15-PGDH enzyme is in a range of equal to or greater than 10 nM and less than 15 nM. “D” represents that IC₅₀ of the inhibitory activity against 15-PGDH enzyme is in a range of equal to or greater than 15 nM and less than 30 nM. “E” represents that IC₅₀ of the inhibitory activity against 15-PGDH enzyme is in a range of greater than 30 nM.

It was found via the tests that the IC₅₀ value of the inhibitory activity of the compounds in the present application against 15-PGDH enzyme is less than 100 nM; the IC₅₀ value of the inhibitory activity of some compounds in the present application against 15-PGDH enzyme is equal to or greater than 20 nM and less than 50 nM; the IC₅₀ value of the inhibitory activity of some compounds in the present application against 15-PGDH enzyme is equal to or greater than 10 nM and less than 20 nM; the IC₅₀ value of the inhibitory activity of some compounds in the present application against 15-PGDH enzyme is equal to or greater than 3 nM and less than 10 nM; the IC₅₀ value of the inhibitory activity of some compounds in the present application against 15-PGDH enzyme is equal to or greater than 1.5 nM and less than 3 nM; the IC₅₀ value of the inhibitory activity of some compounds in the present application against 15-PGDH enzyme is less than 1.5 nM.

### Test Example 2: Assay of Intracellular PGE2 Up-Regulatory Activity

### 1. Experimental Materials:

| Reagents/Materials/Instruments | Manufacturer | Item No. |
|---|---|---|
| F12k Kaighn's Modification | Hyclone Laboratories, Inc | SH3052601/AG29722854 |
| culture medium | | |
| TRYPSIN | Hyclone Laboratories, Inc | J190002 |
| Fetal Bovine Serum | PAN-Biotech | ST-30-3302 |
| Penicillin-Streptomycin | Hyclone Laboratories, Inc | J190007 |
| DMSO | Sigma-Aldrich Corporation | D8418 |
| A549 cells | Nanjing Cobioer | CBP60084 |
| | Biosciences Co., Ltd. | |
| Prostaglandin E2 Kit | PerkinElmer Corporation | 62P2APEG |
| 24-well plate | Corning United States | 3337 |
| | Corporation | |
| 384-well plate | Corning United States | 3570 |
| | Corporation | |
| Multifunctional | BMG LABTECH | PHERAstar ® FSX |
| microplate reader | Corporation | |
| CO₂ cell incubator | Thermo Fisher Scientific | RI-250 |
| | Corporation | |
| Microscope | Thermo Fisher Scientific | DMI1 |
| | Corporation | |

### 2. Experimental Method:

a) A549 cells were inoculated in the 24-well plate, and after cell adhesion, IL-1β was added thereto for 16 h of stimulation to induce COX2 expression and PGE2 production;
b) A solution of the compound to be tested was prepared with F12k Kaighn's Modification culture medium and serially diluted to 7 concentrations of 0.64 nM, 3.2 nM, 16 nM, 80 nM, 400 nM, 2000 nM, and 10000 nM, and meanwhile the positive control group and negative control group were set up; the cell supernatants were collected after 8 h of action, in which the positive control group was induced by IL-1β without treatment of the compounds, and the negative control group was neither stimulated by IL-1β, nor treated with the compounds; c) The PGE2 content of the samples was determined by Prostaglandin E2 Kit, and the fluorescence signal was detected by a multifunctional microplate reader (Ex/Em=337/620, 337/665).

### 3. Data Analysis:

a) A standard curve was plotted with the PGE2 standard in the Prostaglandin E2 Kit, and the PGE2 concentration was calculated by substituting with the fluorescence signal of the sample.
b) The PGE2 up-regulation rate % was calculated by using the following formula:

PGE2 up-regulation rate %=(PGE2 concentration of sample group/PGE2 concentration of positive control group)×100%.

### 4. Experimental Results:

The compounds of the present application, particularly the compounds prepared in Examples 1-26, are able to achieve a PGE2 up-regulation rate of greater than 100% in A549 cells. The compounds of the present application, particularly the compounds prepared in Examples 1-26, have good intracellular PGE2 up-regulatory activity.

For the purpose of describing and disclosing, all patents, patent applications and other established publications are expressly incorporated herein by reference. These publications are provided solely for their disclosure prior to the filing date of this application. All statements regarding the dates of these documents or the representation of the contents of these documents are based on the information available to the applicants and do not constitute any admission as to the correctness of the dates of these documents or the contents of these documents. Moreover, any reference to these publications herein does not constitute an admission that the publications form part of the common general knowledge in the art in any country.

Those skilled in the art will recognize that the scope of the present application is not limited to the various specific embodiments and examples described above, but is capable of making various modifications, substitutions, or recombinations without departing from the spirit of the present application, and that these adjusted technical solutions fall within the protection scope of the present application.

1. A compound represented by formula (I), a stereoisomer, tautomer or mixture form thereof, or a pharmaceutically acceptable salt thereof, or a solvate thereof, or a prodrug thereof: 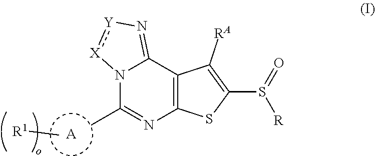
ring A is selected from the group consisting of an aromatic ring, an aromatic heterocycle, an unsaturated aliphatic heterocycle, a fused ring consisting of an aromatic ring and an unsaturated aliphatic heterocycle, and a fused ring consisting of an aromatic heterocycle and an unsaturated aliphatic heterocycle;
R is selected from the group consisting of a C₁-C₁₀ chain hydrocarbyl, a 3-12 membered alicyclic ring, and a 3-12 membered aliphatic heterocycle, wherein R is substituted by 0-2 R², the R² is each independently selected from the group consisting of deuterium, tritium, nitro, hydroxy, an aldehyde group, halogen, cyano, —C(O)OR^{a}, —OC(O)R^{b}, —C(O)NHR^{X}, —NHC(O)R^{Y}, ═O, C₁-C₆ alkoxy, C₁-C₆ haloalkoxy, 3-8 membered cycloalkyl, 3-8 membered heterocycloalkyl, 6-10 membered aromatic ring, and 5-10 membered aromatic heterocycle, wherein R^{a}, R^{b}, R^{X}, R^{Y} are each independently selected from the group consisting of C₁-C₆ alkyl, 3-8 membered cycloalkyl, and 3-8 membered heterocycloalkyl,
o is selected from the group consisting of 0, 1, 2, 3, and 4,
R¹ is each independently selected from the group consisting of deuterium, tritium, nitro, hydroxy, mercapto, halogen, cyano, ═O, imino, an amine group, an ester group, an aldehyde group, carboxyl, amido, C₁-C₆ alkyl, C₁-C₆ haloalkyl, C₁-C₆ alkoxy, C₁-C₆ haloalkoxy, 3-12 membered cycloalkyl, and 3-12 membered heterocycloalkyl, [symbol]
is a single bond or a double bond, and when [symbol] is a double bond, X and Y are each independently CR^{B} or N; when [symbol] is a single bond, X and Y are each independently CR^{C}R^{D}, or NR^{E},
R^{A}, R^{B}, R^{C}, R^{D}, R^{E} are each independently selected from the group consisting of hydrogen, hydroxy, halogen, an amine group, cyano, C₁-C₆ alkyl, C₁-C₆ alkoxy, C₁-C₆ haloalkyl, and 3-8 membered cycloalkyl;
the aromatic heterocycle, the aliphatic heterocycle, the unsaturated aliphatic heterocycle, the fused ring, the heterocycloalkyl each independently comprise 1-3 heteroatoms which are independently selected from the group consisting of N, O, and S;
the R1 is optionally substituted by one or two or more independently selected from the group consisting of deuterium, tritium, nitro, hydroxy, an aldehyde group, an amine group, imino, halogen, cyano, an ester group, carboxyl, amido, ═O, C₁-C₆ alkyl, C₁-C₆ alkoxy, 3-8 membered cycloalkyl, 6-10 membered aromatic ring, and 5-10 membered aromatic heterocycle.

2. The compound, the stereoisomer, tautomer or mixture thereof, or the pharmaceutically acceptable salt thereof, or the solvate thereof, or the prodrug thereof according to claim 1, wherein the [symbol] is a double bond, and at least one of X and Y is CR^{B};
alternatively, the X is N, and the Y is CR^{B}, wherein R^{B} is selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl;
alternatively, the Y is N, and the X is CR^{B}, wherein R^{B} is selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl;
alternatively, both the X and the Y are CR^{B}, wherein R^{B} is each independently selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl.

3. The compound, the stereoisomer, tautomer or mixture thereof, or the pharmaceutically acceptable salt thereof, or the solvate thereof, or the prodrug thereof according to claim 1, wherein the [symbol] is a single bond, and at least one of X and Y is CR^{C}R^{D};
alternatively, the X is NR^{E}, the Y is CR^{C}R^{D}, wherein R^{C}, R^{D}, R^{E} are each independently selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl;
alternatively, the X is CR^{C}R^{D}, the Y is NR^{E}, wherein R^{C}, R^{D}, R^{E} are each independently selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl;
alternatively, both the X and the Y are CR^{C}R^{D}, wherein R^{C}, R^{D} are each independently selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl.

4. The compound, the stereoisomer, tautomer or mixture thereof, or the pharmaceutically acceptable salt thereof, or the solvate thereof, or the prodrug thereof according to claim 1, wherein the R^{A} is selected from the group consisting of hydrogen, hydroxy, cyano, fluoro, chloro, bromo, —NH₂, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclopropylmethyl, cyclobutyl, cyclopentyl, and cyclohexyl.

5. The compound, the stereoisomer, tautomer or mixture thereof, or the pharmaceutically acceptable salt thereof, or the solvate thereof, or the prodrug thereof according to claim 1, wherein the R is selected from the group consisting of C₁-C₁₀ alkyl, 3-12 membered cycloalkyl, and 3-12 membered heterocycloalkyl, wherein R is substituted by 0 or 1 R².

6. The compound, the stereoisomer, tautomer or mixture thereof, or the pharmaceutically acceptable salt thereof, or the solvate thereof, or the prodrug thereof according to claim 1, wherein the compound is represented by formula (II), formula (III) or formula (IV): 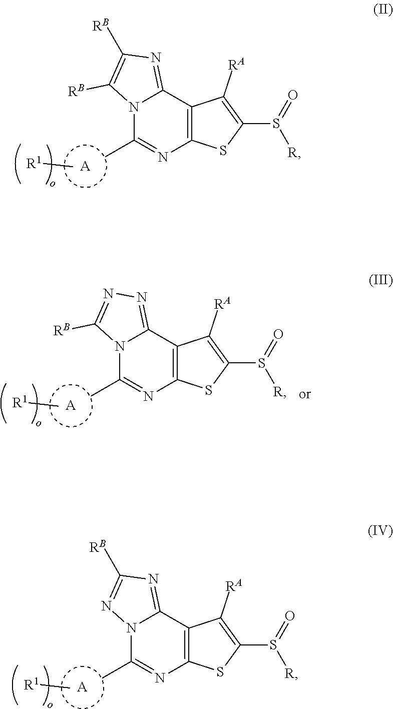
R^{B} is each independently selected from the group consisting of hydrogen, hydroxy, cyano, halogen, methyl, ethyl, n-propyl, isopropyl, n-butyl, isobutyl, tert-butyl, methoxy, ethoxy, n-propoxy, isopropoxy, n-butoxy, isobutoxy, tert-butoxy, trifluoromethyl, trifluoroethyl, trichloromethyl, trichloroethyl, cyclopropyl, cyclobutyl, cyclopentyl, and cyclohexyl,
R is selected from the group consisting of C₁-C₁₀ alkyl, 3-12 membered cycloalkyl, and 3-12 membered heterocycloalkyl, wherein R is substituted by 0 or 1 R², the R² is selected from the group consisting of deuterium, tritium, nitro, hydroxy, an aldehyde group, halogen, cyano, —C(O)OR^{a}, —OC(O)R^{b}, —C(O)NHR^{X}, —NHC(O)R^{Y}, ═O, C₁-C₆ alkoxy, C₁-C₆ haloalkoxy, 3-8 membered cycloalkyl, 3-8 membered heterocycloalkyl, a 6-10 membered aromatic ring, and a 5-10 membered aromatic heterocycle, wherein R^{a}, R^{b}, R^{X}, R^{Y} are each independently selected from the group consisting of C₁-C₆ alkyl, 3-8 membered cycloalkyl, and 3-8 membered heterocycloalkyl.

7. The compound, the stereoisomer, tautomer or mixture thereof, or the pharmaceutically acceptable salt thereof, or the solvate thereof, or the prodrug thereof according to claim 1, wherein the R is selected from the group consisting of C₁-C₁₀ alkyl, 3-12 membered cycloalkyl, and 3-12 membered heterocycloalkyl, the alkyl is a linear or branched alkyl, the cycloalkyl and the heterocycloalkyl are monocyclic or bicyclic, and the heterocycloalkyl comprises 1 heteroatom which is selected from the group consisting of N, O, and S, wherein R is substituted by 0 or 1 R².

8. The compound, the stereoisomer, tautomer or mixture thereof, or the pharmaceutically acceptable salt thereof, or the solvate thereof, or the prodrug thereof according to claim 7, wherein the R² is selected from the group consisting of deuterium, tritium, nitro, hydroxy, an aldehyde group, halogen, cyano, methoxy, ethoxy, n-propoxy, isopropoxy, n-butoxy, isobutoxy, tert-butoxy, trifluoromethoxy, trifluoroethoxy, trifluoropropoxy, —C(O)OCH₃, —C(O)OC₂H₅, —OC(O)CH₃, —OC(O)C₂H₅, —C(O)NHCH₃, —C(O)NHC₂H₅, —NHC(O)CH₃, —NHC(O)C₂H₅, ═O,

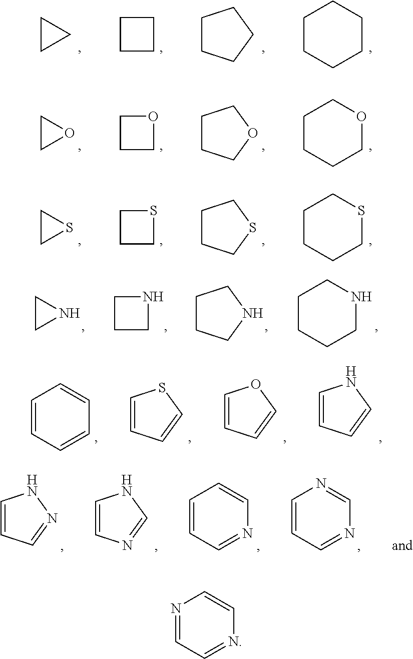

9. The compound, the stereoisomer, tautomer or mixture thereof, or the pharmaceutically acceptable salt thereof, or the solvate thereof, or the prodrug thereof according to claim 1, wherein the ring A is selected from the group consisting of a 6-10 membered aromatic ring, a 5-10 membered aromatic heterocycle, a 3-8 membered unsaturated aliphatic heterocycle, a 7-12 membered fused ring consisting of an aromatic ring and an unsaturated aliphatic heterocycle, and a 7-12 membered fused ring consisting of an aromatic heterocycle and an unsaturated aliphatic heterocycle.

10. The compound, the stereoisomer, tautomer or mixture form thereof, or the pharmaceutically acceptable salt thereof, or the solvate thereof, or the prodrug thereof according to claim 9, wherein ring A is selected from the group consisting of

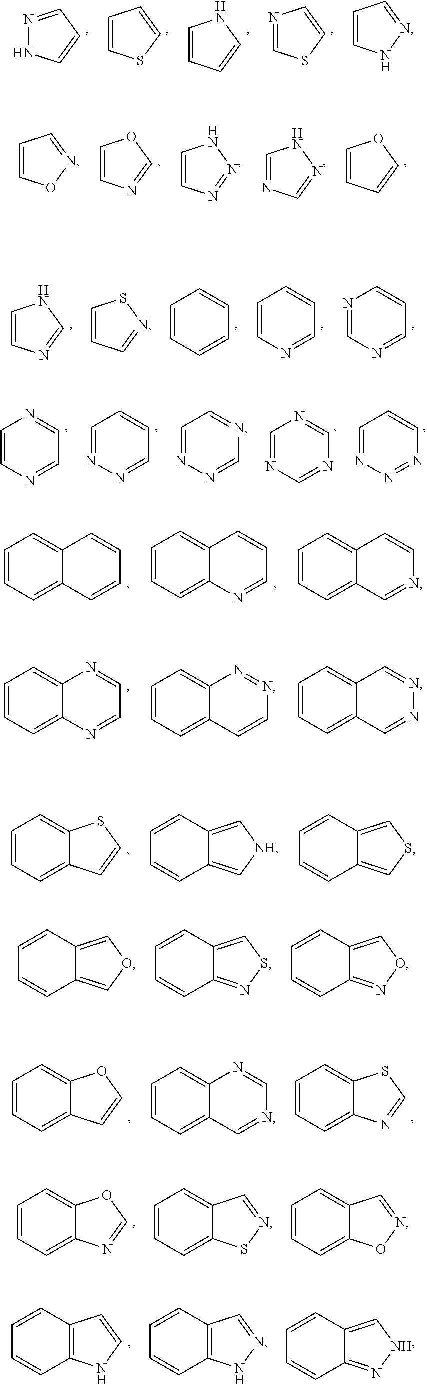

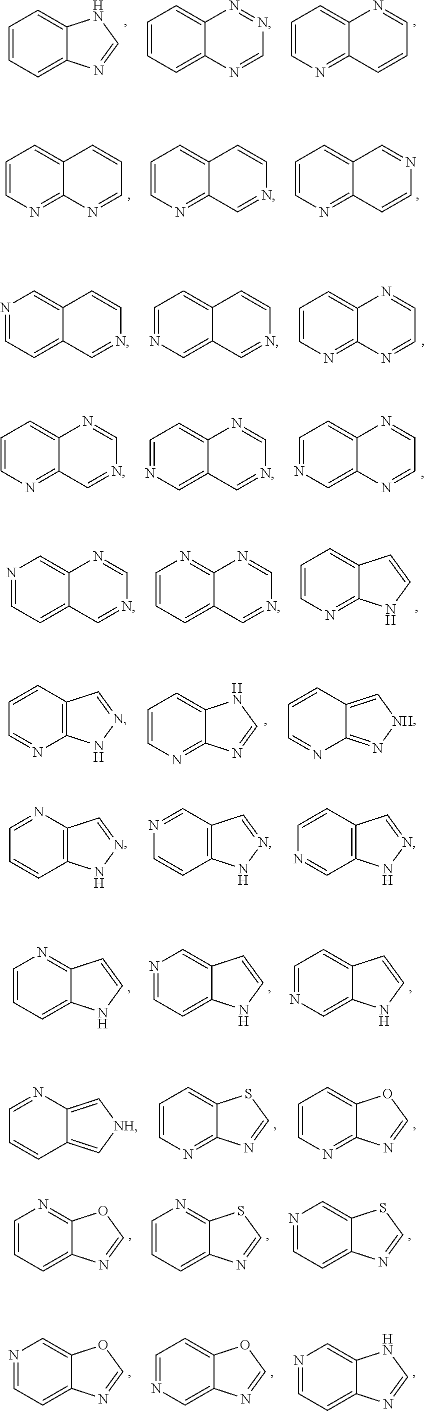

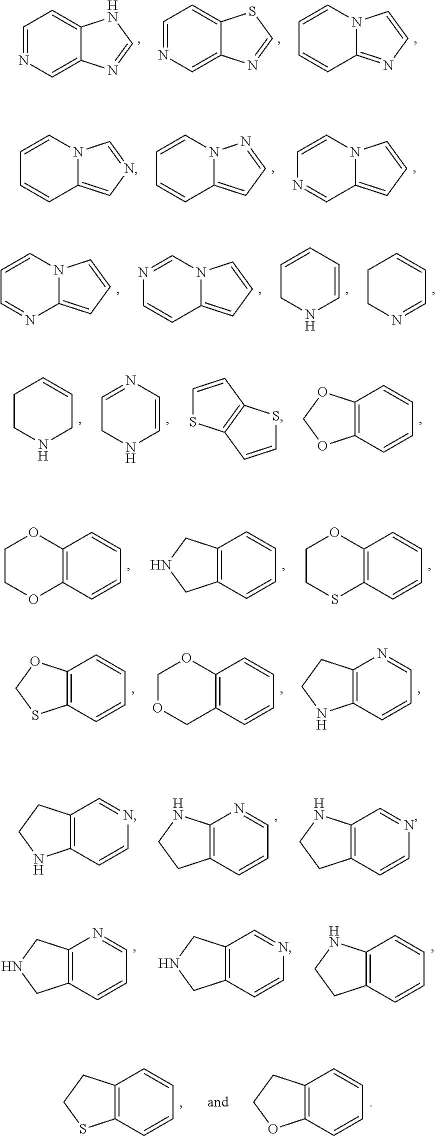

11. The compound, the stereoisomer, tautomer or mixture form thereof, or the pharmaceutically acceptable salt thereof, or the solvate thereof, or the prodrug thereof according to claim 1, wherein
R¹ is each independently selected from the group consisting of deuterium, tritium, nitro, hydroxy, mercapto, halogen, cyano, ═O, imino, an amine group, an ester group, an aldehyde group, carboxyl, amido, cyclopropyl, cyclopropylmethyl, cyclobutyl, cyclopentyl, cyclohexyl, methyl, trifluoromethyl, ethyl, n-propyl, isopropyl, n-butyl, isobutyl, tert-butyl, n-pentyl, isopentyl, tert-pentyl, n-hexyl, morpholinyl, thiomorpholinyl, piperidinyl, piperazinyl, oxazolidinyl, isoxazolidinyl, thiazolidinyl, isothiazolidinyl, dioxolanyl, dioxanyl, methoxy, ethoxy, n-propoxy, isopropoxy, cyclopropoxy, cyclopropylmethoxy, n-butoxy, isobutoxy, tert-butoxy, n-pentyloxy, isopentyloxy, tert-pentyloxy, and n-hexyloxy, wherein the R¹ is optionally substituted by one or more independently selected from the group consisting of deuterium, tritium, nitro, hydroxy, —NH₂, mercapto, halogen, cyano, an ester group, carboxyl, amido, ═O, ═NH, C₁-C₆ alkyl, C₁-C₆ haloalkyl, C₁-C₆ alkoxy, C₁-C₆ haloalkoxy, 3-8 membered cycloalkyl, a 5-10 membered aliphatic heterocycle, a 6-10 membered aromatic ring and a 5-10 membered aromatic heterocycle.

12. The compound, the stereoisomer, tautomer or mixture form thereof, or the pharmaceutically acceptable salt thereof, or the solvate thereof, or the prodrug thereof according to claim 1, wherein
the ring A is selected from the group consisting of phenyl, naphthyl, a 5-8 membered aromatic heterocycle comprising at least one heteroatom of N or O or S, a 5-8 membered unsaturated aliphatic heterocycle comprising at least one heteroatom of N or O or S, a 9-12 membered fused ring consisting of phenyl and the 5-8 membered unsaturated aliphatic heterocycle, and an 8-14 membered fused ring consisting of the 5-8 membered aromatic heterocycle and the 5-8 membered unsaturated aliphatic heterocycle;
the R is selected from the group consisting of C₁-C₁₀ alkyl, a 4-6 membered cycloalkyl, and a 3-6 membered heterocycloalkyl comprising at least one heteroatom of O or S, wherein R is substituted by 0-1 R², and the R² is each independently selected from the group consisting of deuterium, tritium, hydroxy, —C(O)OR^{a}, phenyl, thienyl, furanyl, pyrrolyl, and thiazolyl;
the o is selected from the group consisting of 0, 1, and 2;
the R¹ is each independently selected from the group consisting of ═O, an ester group, carboxyl, C₁-C₆ alkyl, and a 5-6 membered heterocycloalkyl comprising at least one heteroatom of N or O or S; [symbol]
is a double bond, X is CR^{B}, Y is CR^{B} or N, wherein the R^{B} is each independently selected from the group consisting of hydrogen, C₁-C₆ alkyl, and C₁-C₆ haloalkyl;
R^{A} is selected from the group consisting of hydrogen, an amine group, cyano, C₁-C₆ alkyl, C₁-C₆ alkoxy, and C₁-C₆ haloalkyl.

13. The compound, the stereoisomer, tautomer or mixture form thereof, or the pharmaceutically acceptable salt thereof, or the solvate thereof, or the prodrug thereof according to claim 1, wherein the compound is selected from the group consisting of:

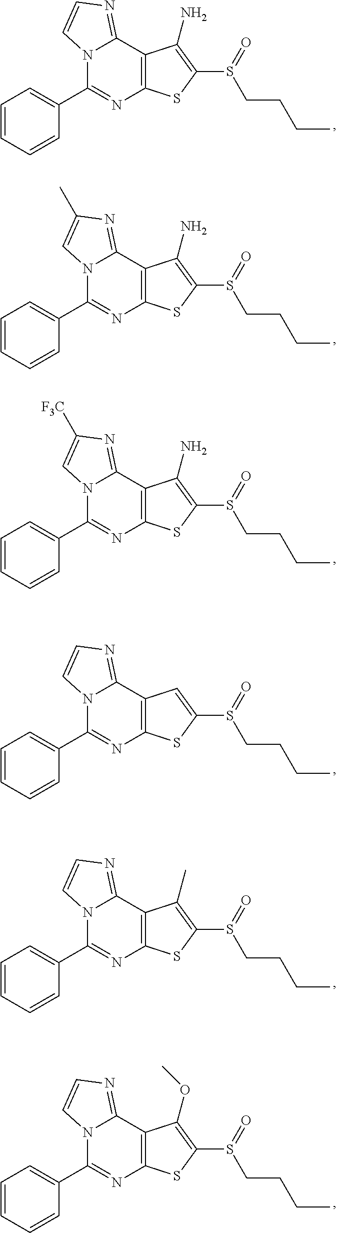

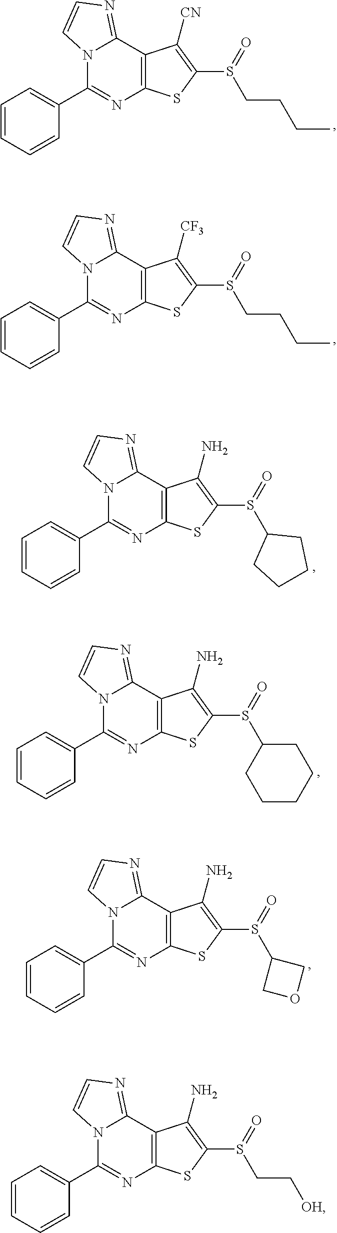 [SMILES: CCCCS(=O)c1sc2nc(-c3ccccc3)n3ccnc3c2c1C#N ; CCCCS(=O)c1sc2nc(-c3ccccc3)n3ccnc3c2c1C(F)(F)F ; Nc1c(S(=O)C2CCCC2)sc2nc(-c3ccccc3)n3ccnc3c12 ; Nc1c(S(=O)C2CCCCC2)sc2nc(-c3ccccc3)n3ccnc3c12 ; Nc1c(S(=O)C2COC2)sc2nc(-c3ccccc3)n3ccnc3c12 ; Nc1c(S(=O)CCO)sc2nc(-c3ccccc3)n3ccnc3c12]

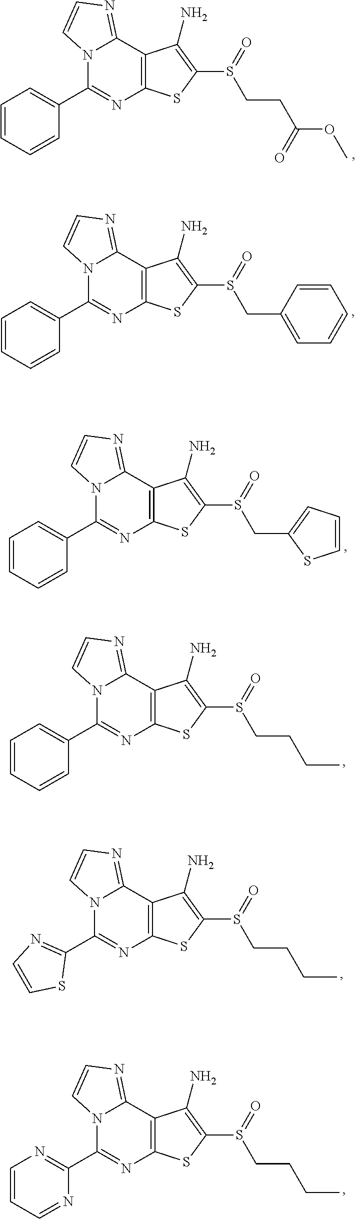 [SMILES: COC(=O)CCS(=O)c1sc2nc(-c3ccccc3)n3ccnc3c2c1N ; Nc1c(S(=O)Cc2ccccc2)sc2nc(-c3ccccc3)n3ccnc3c12 ; Nc1c(S(=O)Cc2cccs2)sc2nc(-c3ccccc3)n3ccnc3c12 ; CCCCS(=O)c1sc2nc(-c3ccccc3)n3ccnc3c2c1N ; CCCCS(=O)c1sc2nc(-c3nccs3)n3ccnc3c2c1N ; CCCCS(=O)c1sc2nc(-c3ncccn3)n3ccnc3c2c1N]

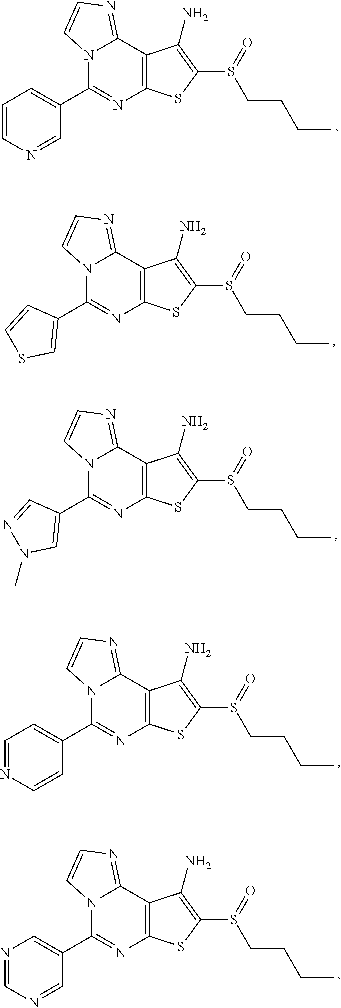 [SMILES: CCCCS(=O)c1sc2nc(-c3cccnc3)n3ccnc3c2c1N ; CCCCS(=O)c1sc2nc(-c3ccsc3)n3ccnc3c2c1N ; CCCCS(=O)c1sc2nc(-c3cnn(C)c3)n3ccnc3c2c1N ; CCCCS(=O)c1sc2nc(-c3ccncc3)n3ccnc3c2c1N ; CCCCS(=O)c1sc2nc(-c3cncnc3)n3ccnc3c2c1N]

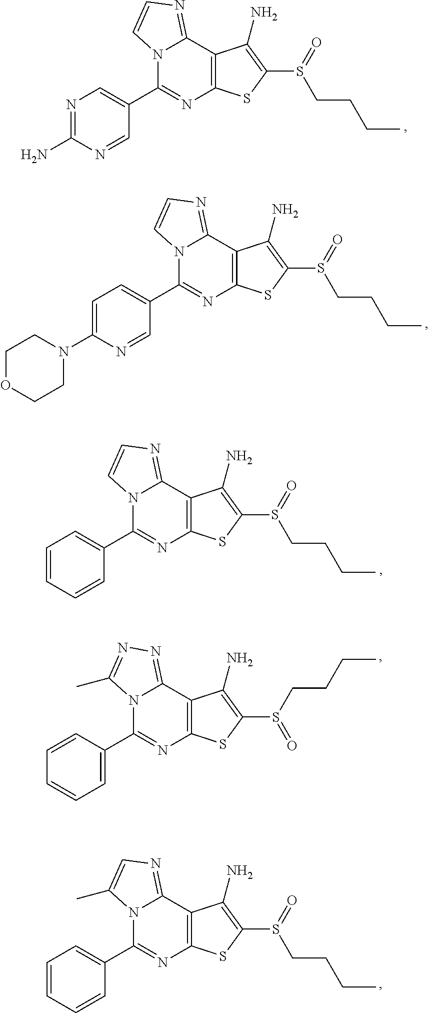 [SMILES: CCCCS(=O)c1sc2nc(-c3cnc(N)nc3)n3ccnc3c2c1N ; CCCCS(=O)c1sc2nc(-c3ccc(N4CCOCC4)nc3)n3ccnc3c2c1N ; CCCCS(=O)c1sc2nc(-c3ccccc3)n3ccnc3c2c1N ; CCCCS(=O)c1sc2nc(-c3ccccc3)n3c(C)nnc3c2c1N ; CCCCS(=O)c1sc2nc(-c3ccccc3)n3c(C)cnc3c2c1N]

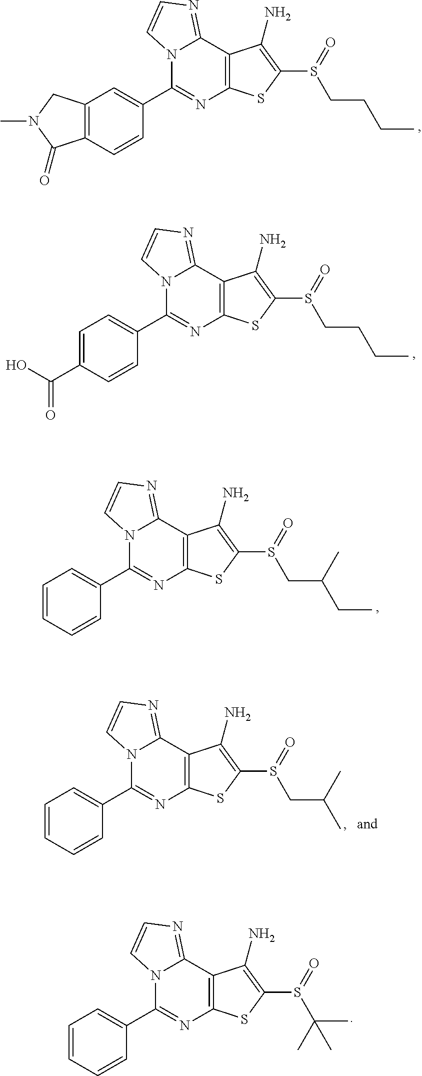 [SMILES: CCCCS(=O)c1sc2nc(-c3ccc4c(c3)CN(C)C4=O)n3ccnc3c2c1N ; CCCCS(=O)c1sc2nc(-c3ccc(C(=O)O)cc3)n3ccnc3c2c1N ; CCC(C)CS(=O)c1sc2nc(-c3ccccc3)n3ccnc3c2c1N ; CC(C)CS(=O)c1sc2nc(-c3ccccc3)n3ccnc3c2c1N ; CC(C)(C)S(=O)c1sc2nc(-c3ccccc3)n3ccnc3c2c1N]

14. A pharmaceutical composition, comprising at least one of the compound, the stereoisomer, tautomer or mixture form thereof, or the pharmaceutically acceptable salt thereof, or the solvate thereof, or the prodrug thereof according to claim 1, and at least one pharmaceutically acceptable excipient.

15. (canceled)

16. The compound, the stereoisomer, tautomer or mixture thereof, or the pharmaceutically acceptable salt thereof, or the solvate thereof, or the prodrug thereof according to claim 7, wherein the R is selected from the group consisting of methyl, ethyl, n-propyl, isopropyl, n-butyl, isobutyl, tert-butyl, n-pentyl, n-hexyl, cyclopropyl, cyclobutyl, cyclopentyl, cyclohexyl, cycloheptyl, cyclooctyl, 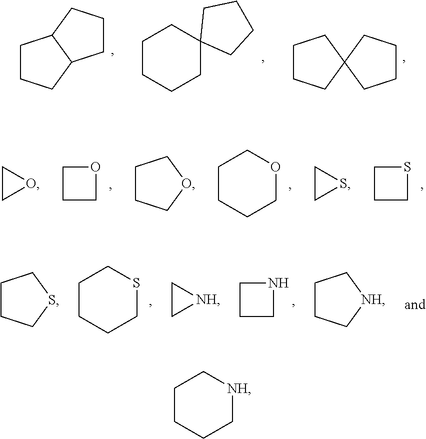
wherein R is substituted by 0 or 1 R²;
the R² is selected from the group consisting of deuterium, tritium, nitro, hydroxy, an aldehyde group, halogen, cyano, —C(O)OR^{a}, —OC(O)R^{b}, —C(O)NRx, —NHC(O)R^{Y}, ═O, C₁-C₆ alkoxy, C₁-C₆ haloalkoxy, 3-8 membered cycloalkyl, 3-8 membered heterocycloalkyl, a 6-10 membered aromatic ring, and a 5-10 membered aromatic heterocycle, wherein R^{a}, R^{b}, R^{X}, R^{Y} are each independently selected from the group consisting of C₁-C₆ alkyl, 3-8 membered cycloalkyl, and 3-8 membered heterocycloalkyl.

17. The compound, the stereoisomer, tautomer or mixture thereof, or the pharmaceutically acceptable salt thereof, or the solvate thereof, or the prodrug thereof according to claim 9, wherein the aromatic ring and the aromatic heterocycle are a monocyclic ring or a fused ring, the unsaturated aliphatic heterocycle is monocyclic, the fused ring is bicyclic, and the aromatic heterocycle, the unsaturated aliphatic heterocycle and the fused ring each independently comprise 1-2 heteroatoms which are independently selected from the group consisting of N, O, and S.

18. The compound, the stereoisomer, tautomer or mixture thereof, or the pharmaceutically acceptable salt thereof, or the solvate thereof, or the prodrug thereof according to claim 12, wherein
the ring A is selected from the group consisting of 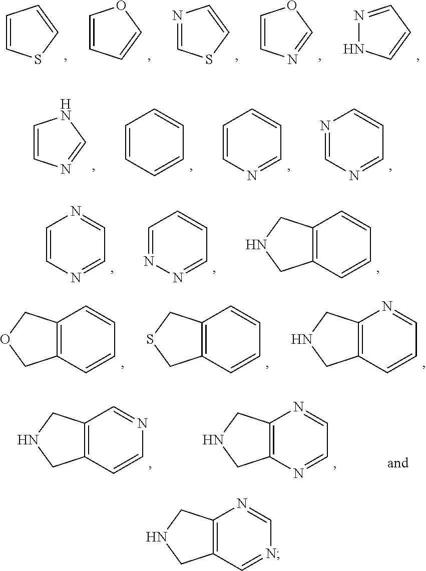
the R is selected from the group consisting of C₁-C₆ alkyl, a 4-6 membered cycloalkyl, and a 4-6 membered heterocycloalkyl comprising 1 O atom, wherein R is substituted by 0-1 R², and the R² is independently hydroxy or phenyl;
the o is selected from the group consisting of 0, 1, and 2;
the R¹ is each independently selected from the group consisting of ═O, carboxyl, C₁-C₃ alkyl, and a 5-6 membered heterocycloalkyl comprising at least one heteroatom of N or O or S; [symbol]
is a double bond, X is CR^{B}, Y is CR^{B} or N, wherein the R^{B} is each independently selected from the group consisting of hydrogen, C₁-C₆ alkyl, and C₁-C₆ haloalkyl;
R^{A} is selected from the group consisting of hydrogen, an amine group, cyano, C₁-C₆ alkyl, C₁-C₆ alkoxy, and C₁-C₆ haloalkyl.

19. A method of treating or preventing a disease associated with 15-PGDH, comprising administering to a subject in need thereof the compound, the stereoisomer, tautomer or mixture thereof, or the pharmaceutically acceptable salt thereof, or the solvate thereof, or the prodrug thereof according to claim 1, or a pharmaceutical composition comprising the same.

20. The method according to claim 19, wherein the method is for treating or preventing fibrosis, oral ulcer, gum disease, colitis, ulcerative colitis, gastroduodenal ulcer, inflammatory disease, vascular insufficiency, Raynaud's disease, Buerger's disease, neuropathy, pulmonary arterial hypertension, cardiovascular and renal disease, cardiovascular disease, trauma, skin damage, autoimmune disease, graft-versus-host disease, osteoporosis, ear disease, eye disease, neutropenia, diabetes mellitus, underactive bladder; or for promoting hair growth, pigmentation, tissue repair, tissue regeneration, implant in stem cell transplantation or bone marrow transplantation or organ transplantation, neurogenesis and neuronal cell death, or muscle regeneration, and cervical ripening; or for resistance to the toxicity of chemotherapy and the toxicity of immunosuppressant.
